SEQUENCE LISTING (74 sequences)

SEQ ID NO: 1 - DNA, 75 bp, Artificial Sequence
  iNKT TCR-alpha chain cloned sequence
gtgggcgatagaggttcagccttagggaggctgcattttggagctgggactcagctgattgtcatacctgacatc

SEQ ID NO: 2 - DNA, 75 bp, Artificial Sequence
  iNKT TCR-beta chain cloned sequence
gccagcggtgatgctcggggggggggaaataccctctattttggaaaaggaagccggctcattgttgtagaggat

SEQ ID NO: 3 - DNA, 75 bp, Artificial Sequence
  iNKT TCR-beta chain cloned sequence
gccagcggggggacagtccattctggaaatacgctctattttggagaaggaagccggctcattgttgtagaggat

SEQ ID NO: 4 - DNA, 75 bp, Artificial Sequence
  iNKT TCR-beta chain cloned sequence
gccagcggtgatacgggacaaacaaacacagaagtcttctttggtaaaggaaccagactcacagttgtagaggat

SEQ ID NO: 5 - DNA, 72 bp, Artificial Sequence
  iNKT TCR-beta chain cloned sequence
gccagcggtgaggggacagcaaacacagaagtcttctttggtaaaggaaccagactcacagttgtagaggat

SEQ ID NO: 6 - DNA, 69 bp, Artificial Sequence
  iNKT TCR-beta chain cloned sequence
gccagcggtgaggcagggaacacagaagtcttctttggtaaaggaaccagactcacagttgtagaggat

SEQ ID NO: 7 - DNA, 78 bp, Artificial Sequence
  iNKT TCR-alpha chain cloned sequence
gtgagcgacagaggctcaaccctggggaggctatactttggaagaggaactcagttgactgtctggcctgatatccag

SEQ ID NO: 8 - DNA, 75 bp, Artificial Sequence
  iNKT TCR-beta chain cloned sequence
agcagtgacctccgaggacagaacacagatacgcagtattttggcccaggcacccggctgacagtgctcgaggac

SEQ ID NO: 9 - DNA, 78 bp, Artificial Sequence
  iNKT TCR-beta chain cloned sequence
agcagtgaattaaaggaaacaggggttcaagagacccagtacttcgggccaggcacgcggctcctggtgctcgaggac

SEQ ID NO: 10 - DNA, 69 bp, Artificial Sequence
  iNKT TCR-beta chain cloned sequence
agcagtgtatctcagggcggcactgaagctttctttggacaaggcaccagactcacagttgtagaggac

SEQ ID NO: 11 - DNA, 69 bp, Artificial Sequence; identical to SEQ ID NO: 10
  iNKT TCR-beta chain cloned sequence

SEQ ID NO: 12 - DNA, 72 bp, Artificial Sequence
  iNKT TCR-beta chain cloned sequence
agcagtgaccggacaggcgtgaacactgaagctttctttggacaaggcaccagactcacagttgtagaggac

SEQ ID NO: 13 - DNA, 72 bp, Artificial Sequence
  iNKT TCR-beta chain cloned sequence
agcagtgaaccggacagggggggggctgaagctttctttggacaaggcaccagactcacagttgtagaggac

SEQ ID NO: 14 - DNA, 32 bp, Artificial Sequence
  iNKT TCR-apha chain forward primer
gggagatactcagcaactctggataaagatgc

SEQ ID NO: 15 - DNA, 27 bp, Artificial Sequence
  iNKT TCR-apha chain reverse primer
ccagattccatggttttcggcacattg

SEQ ID NO: 16 - DNA, 30 bp, Artificial Sequence
  iNKT TCR-beta chain forward primer
ggagatatccctgatggatacaaggcctcc

SEQ ID NO: 17 - DNA, 30 bp, Artificial Sequence
  iNKT TCR-beta chain reverse primer
gggtagccttttgtttgtttgcaatctctg

SEQ ID NO: 18 - DNA, 831 bp, Artificial Sequence
  Human iNKT TCR-alpha chain cDNA
atgaaaaagcatctgacgaccttcttggtgattttgtggctttatttttatagggggaatggcaaaaaccaagtggagcagagtcctcagtccctgatcatcctggagggaaagaactgcactcttcaatgcaattatacagtgagccccttcagcaacttaaggtggtataagcaagatactgggagaggtcctgtttccctgacaatcatgactttcagtgagaacacaaagtcgaacggaagatatacagcaactctggatgcagacacaaagcaaagctctctgcacatcacagcctcccagctcagcgattcagcctcctacatctgtgtggtgagcgacagaggctcaaccctggggaggctatactttggaagaggaactcagttgactgtctggcctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 19 - DNA, 831 bp, Artificial Sequence
  Human iNKT TCR-alpha chain cDNA codon-optimized
atgaaaaagcatctgacaacattcctggtcattctgtggctgtacttctaccgaggcaacggcaaaaatcaggtggagcagtccccacagtccctgatcattctggaggggaagaactgcactctgcagtgtaattacaccgtgtctccctttagtaacctgcgctggtataaacaggacaccggacgaggacccgtgagcctgacaatcatgactttctcagagaacacaaagagcaatggacggtacaccgctacactggacgcagataccaaacagagctccctgcacatcacagcatctcagctgtcagatagcgcctcctacatttgcgtggtctctgaccgagggagtaccctgggccgactgtattttggaagggggacccagctgacagtgtggcccgacatccagaacccagatcccgccgtctaccagctgcgcgacagcaagtctagtgataaaagcgtgtgcctgttcacagactttgattctcagactaatgtctctcagagtaaggacagtgacgtgtacattactgacaaaaccgtcctggatatgaggagcatggacttcaagtcaaacagcgccgtggcttggtcaaacaagagcgacttcgcatgcgccaatgcttttaacaattcaatcattccagaggataccttctttcctagcccagaatcaagctgtgacgtgaagctggtcgagaaaagtttcgaaactgataccaacctgaattttcagaacctgtctgtgatcggcttcagaatcctgctgctgaaggtcgccggctttaatctgctgatgacactgagactgtggtcctcttga

SEQ ID NO: 20 - protein, 276 aa, Artificial Sequence
  Human iNKT TCR-alpha chain
MKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYICVVSDRGSTLGRLYFGRGTQLTVWPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 21 - DNA, 336 bp, Artificial Sequence
  Human iNKT TCR-beta chain cDNA (before D/J/N region)
atgactatcaggctcctctgctacatgggcttttattttctgggggcaggcctcatggaagctgacatctaccagaccccaagataccttgttatagggacaggaaagaagatcactctggaatgttctcaaaccatgggccatgacaaaatgtactggtatcaacaagatccaggaatggaactacacctcatccactattcctatggagttaattccacagagaagggagatctttcctctgagtcaacagtctccagaataaggacggagcattttcccctgaccctggagtctgccaggccctcacatacctctcagtacctctgtgccagc

SEQ ID NO: 22 - DNA, 336 bp, Artificial Sequence
  Human iNKT TCR-beta chain cDNA codon-optimized
atgaccatccggctgctgtgctacatgggcttctattttctgggggcaggcctgatggaagccgacatctaccagactcccagatacctggtcatcggaaccgggaagaaaattacactggagtgttcccagacaatgggccacgataagatgtactggtatcagcaggaccctgggatggaactgcacctgatccattactcctatggcgtgaactctaccgagaagggcgacctgagcagcgaatccaccgtctctcgaattaggacagagcactttcctctgactctggaaagcgcccgaccaagtcatacatcacagtacctgtgcgctagc

SEQ ID NO: 23 - protein, 112 aa, Artificial Sequence
  Human iNKT TCR-beta chain
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCAS

SEQ ID NO: 24 - DNA, 60 bp, Artificial Sequence
  Human iNKT TCR Beta Chain Diverse Region (D/J/N)
gtagcggttgggccccaagagacccagtacttcgggccaggcacgcggctcctggtgctc

SEQ ID NO: 25 - DNA, 60 bp, Artificial Sequence
  Human iNKT TCR Beta Chain Diverse Region (D/J/N)
gtggcagtcggacctcaggagacccagtacttcggacccggcacccgcctgctggtgctg

SEQ ID NO: 26 - protein, 20 aa, Artificial Sequence
  Human iNKT TCR Beta Chain Diverse Region (D/J/N)
VAVGPQETQYFGPGTRLLVL

SEQ ID NO: 27 - DNA, 54 bp, Artificial Sequence
  Human iNKT TCR Beta Chain Diverse Region (D/J/N)
agtgggccagggtacgagcagtacttcgggccgggcaccaggctcacggtcaca

SEQ ID NO: 28 - DNA, 54 bp, Artificial Sequence
  Human iNKT TCR Beta Chain Diverse Region (D/J/N)
tcaggacccggctacgagcagtatttcggccccggaactcggctgaccgtgacc

SEQ ID NO: 29 - protein, 18 aa, Artificial Sequence
  Human iNKT TCR Beta Chain Diverse Region (D/J/N)
SGPGYEQYFGPGTRLTVT

SEQ ID NO: 30 - DNA, 57 bp, Artificial Sequence
  Human iNKT TCR Beta Chain Diverse Region (D/J/N)
agtccccaattaaacactgaagctttctttggacaaggcaccagactcacagttgta

SEQ ID NO: 31 - DNA, 57 bp, Artificial Sequence
  Human iNKT TCR Beta Chain Diverse Region (D/J/N)
tctccacagctgaacaccgaggccttcttcgggcagggcacaaggcttaccgtggtg

SEQ ID NO: 32 - protein, 19 aa, Artificial Sequence
  Human iNKT TCR Beta Chain Diverse Region (D/J/N)
SPQLNTEAFFGQGTRLTVV

SEQ ID NO: 33 - DNA, 78 bp, Artificial Sequence
  Human iNKT TCR Beta Chain Diverse Region (D/J/N)
agtgaattgcgggcgctcgggcccagctcctataattcacccctccactttgggaacgggaccaggctcactgtgaca

SEQ ID NO: 34 - DNA, 78 bp, Artificial Sequence
  Human iNKT TCR Beta Chain Diverse Region (D/J/N)
tccgaactccgagccctggggcctagctcctacaatagccccctgcactttggcaacggaaccaggctgacggtcacc

SEQ ID NO: 35 - protein, 26 aa, Artificial Sequence
  Human iNKT TCR Beta Chain Diverse Region (D/J/N)
SELRALGPSSYNSPLHFGNGTRLTVT

SEQ ID NO: 36 - DNA, 60 bp, Artificial Sequence
  Human iNKT TCR Beta Chain Diverse Region (D/J/N)
agtgaacaggggactactgcgggagctttctttggacaaggcaccagactcacagttgta

SEQ ID NO: 37 - DNA, 60 bp, Artificial Sequence
  Human iNKT TCR Beta Chain Diverse Region (D/J/N)
tccgaacagggaaccacagcaggagccttcttcggtcagggaacaagactgacagtcgtg

SEQ ID NO: 38 - protein, 20 aa, Artificial Sequence
  Human iNKT TCR Beta Chain Diverse Region (D/J/N)
SEQGTTAGAFFGQGTRLTVV

SEQ ID NO: 39 - DNA, 66 bp, Artificial Sequence
  Human iNKT TCR Beta Chain Diverse Region (D/J/N)
agtgagtcacgacatgcgacaggaaacaccatatattttggagagggaagttggctcactgttgta

SEQ ID NO: 40 - DNA, 66 bp, Artificial Sequence
  Human iNKT TCR Beta Chain Diverse Region (D/J/N)
agcgagagcaggcacgcaaccgggaacaccatatactttggcgagggctcctggctgactgtggtg

SEQ ID NO: 41 - protein, 22 aa, Artificial Sequence
  Human iNKT TCR Beta Chain Diverse Region (D/J/N)
SESRHATGNTIYFGEGSWLTVV

SEQ ID NO: 42 - DNA, 69 bp, Artificial Sequence
  Human iNKT TCR Beta Chain Diverse Region (D/J/N)
agtgtacccgggaacgacaggggcaatgaaaaactgttttttggcagtggaacccagctctctgtcttg

SEQ ID NO: 43 - DNA, 69 bp, Artificial Sequence
  Human iNKT TCR Beta Chain Diverse Region (D/J/N)
tccgtgcctggcaacgatagaggtaacgagaagctgtttttcggatccggcacacagctgtctgtcctg

SEQ ID NO: 44 - protein, 23 aa, Artificial Sequence
  Human iNKT TCR Beta Chain Diverse Region (D/J/N)
SVPGNDRGNEKLFFGSGTQLSVL

SEQ ID NO: 45 - DNA, 72 bp, Artificial Sequence
  Human iNKT TCR Beta Chain Diverse Region (D/J/N)
agtgaaggggggggccttaagctagccaaaaacattcagtacttcggcgccgggacccggctctcagtgctg

SEQ ID NO: 46 - DNA, 72 bp, Artificial Sequence
  Human iNKT TCR Beta Chain Diverse Region (D/J/N)
agtgagggagggggactgaagctggctaagaatattcagtacttcggcgccggcactagactgtctgtgctg

SEQ ID NO: 47 - protein, 24 aa, Artificial Sequence
  Human iNKT TCR Beta Chain Diverse Region (D/J/N)
SEGGGLKLAKNIQYFGAGTRLSVL

SEQ ID NO: 48 - DNA, 69 bp, Artificial Sequence
  Human iNKT TCR Beta Chain Diverse Region (D/J/N)
agtgaattcgcctcttcggtacgtggaaacaccatatattttggagagggaagttggctcactgttgta

SEQ ID NO: 49 - DNA, 69 bp, Artificial Sequence
  Human iNKT TCR Beta Chain Diverse Region (D/J/N)
tctgagttcgcgagcagcgtccggggtaataccatttacttcggggaaggcagctggctgaccgtggtg

SEQ ID NO: 50 - protein, 23 aa, Artificial Sequence
  Human iNKT TCR Beta Chain Diverse Region (D/J/N)
SEFASSVRGNTIYFGEGSWLTVV

SEQ ID NO: 51 - DNA, 60 bp, Artificial Sequence
  Human iNKT TCR Beta Chain Diverse Region (D/J/N)
agtgcggcattaggccgggagacccagtacttcgggccaggcacgcggctcctggtgctc

SEQ ID NO: 52 - DNA, 60 bp, Artificial Sequence
  Human iNKT TCR Beta Chain Diverse Region (D/J/N)
tctgcagcccttggccgagagactcagtacttcggccctggcacaagactgctcgtgctc

SEQ ID NO: 53 - protein, 20 aa, Artificial Sequence
  Human iNKT TCR Beta Chain Diverse Region (D/J/N)
SAALGRETQYFGPGTRLLVL

SEQ ID NO: 54 - DNA, 63 bp, Artificial Sequence
  Human iNKT TCR Beta Chain Diverse Region (D/J/N)
agtgcctccgggggtgaatcctacgagcagtacttcgggccgggcaccaggctcacggtcaca

SEQ ID NO: 55 - DNA, 63 bp, Artificial Sequence
  Human iNKT TCR Beta Chain Diverse Region (D/J/N)
agcgcctccggaggagagtcatacgaacagtatttcggccctggcacacgcctcactgtgacc

SEQ ID NO: 56 - protein, 21 aa, Artificial Sequence
  Human iNKT TCR Beta Chain Diverse Region (D/J/N)
SASGGESYEQYFGPGTRLTVT

SEQ ID NO: 57 - DNA, 90 bp, Artificial Sequence
  Human iNKT TCR Beta Chain Diverse Region (D/J/N)
agcggtcgggtctcggggggcgattccctcatagcgtttctaggccaagagacccagtacttcgggccaggcacgcggctcctggtgctc

SEQ ID NO: 58 - DNA, 90 bp, Artificial Sequence
  Human iNKT TCR Beta Chain Diverse Region (D/J/N)
tcaggacgagtgtccggaggggatagcctcatcgcatttctggggcaggaaactcagtacttcggacccggaacacgcctcctggtgctg

SEQ ID NO: 59 - protein, 30 aa, Artificial Sequence
  Human iNKT TCR Beta Chain Diverse Region (D/J/N)
SGRVSGGDSLIAFLGQETQYFGPGTRLLVL

SEQ ID NO: 60 - DNA, 69 bp, Artificial Sequence; identical to SEQ ID NO: 42
  Human iNKT TCR Beta Chain Diverse Region (D/J/N)

SEQ ID NO: 61 - DNA, 69 bp, Artificial Sequence; identical to SEQ ID NO: 43
  Human iNKT TCR Beta Chain Diverse Region (D/J/N)

SEQ ID NO: 62 - protein, 23 aa, Artificial Sequence
  Human iNKT TCR Beta Chain Diverse Region (D/J/N)
SVPGNDRGNEKLFFGSGTQLSVL

SEQ ID NO: 63 - DNA, 534 bp, Artificial Sequence
  Human iNKT TCR-beta chain cDNA (after D/J/N region)
gaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgccaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 64 - DNA, 534 bp, Artificial Sequence
  Human iNKT TCR-beta chain cDNA codon-optimized (after D/J/N region)
gaggacctgaataaggtgttcccccctgaggtggctgtctttgaaccaagtgaggcagaaatttcacatacacagaaagccaccctggtgtgcctggctaccggcttctttcccgatcacgtggagctgagctggtgggtcaacggcaaggaagtgcatagcggagtctccacagacccacagcccctgaaagagcagcctgctctgaatgattccagatactgcctgtctagtagactgcgggtgtctgccaccttctggcagaacccaaggaatcatttcagatgtcaggtgcagttttatggcctgagcgagaacgatgaatggactcaggacagggctaagccagtgacccagatcgtcagcgcagaggcctggggaagagcagactgcgggtttacaagcgtgagctatcagcagggcgtcctgagcgccacaatcctgtacgaaattctgctgggaaaggccactctgtatgctgtgctggtctccgctctggtgctgatggcaatggtcaagcggaaagatttctga

SEQ ID NO: 65 - protein, 177 aa, Artificial Sequence
  Human iNKT TCR-beta chain (after D/J/N region)
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 66 - DNA, 2845 bp, Homo sapiens
aagtggaggcgtcgcgctggcgggcattcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgtaagcagcatcatggaggtaagtttttgaccttgagaaaatgtttttgtttcactgtcctgaggactatttatagacagctctaacatgataaccctcactatgtggagaacattgacagagtaacattttagcagggaaagaagaatcctacagggtcatgttcccttctcctgtggagtggcatgaagaaggtgtatggccccaggtatggccatattactgaccctctacagagagggcaaaggaactgccagtatggtattgcaggataaaggcaggtggttacccacattacctgcaaggctttgatctttcttctgccatttccacattggacatctctgctgaggagagaaaatgaaccactcttttcctttgtataatgttgttttattcttcagacagaagagaggagttatacagctctgcagacatcccattcctgtatggggactgtgtttgcctcttagaggttcccaggccactagaggagataaagggaaacagattgttataacttgatataatgatactataatagatgtaactacaaggagctccagaagcaagagagagggaggaacttggacttctctgcatctttagttggagtccaaaggcttttcaatgaaattctactgcccagggtacattgatgctgaaaccccattcaaatctcctgttatattctagaacagggaattgatttgggagagcatcaggaaggtggatgatctgcccagtcacactgttagtaaattgtagagccaggacctgaactctaatatagtcatgtgttacttaatgacggggacatgttctgagaaatgcttacacaaacctaggtgttgtagcctactacacgcataggctacatggtatagcctattgctcctagactacaaacctgtacagcctgttactgtactgaatactgtgggcagttgtaacacaatggtaagtatttgtgtatctaaacatagaagttgcagtaaaaatatgctattttaatcttatgagaccactgtcatatatacagtccatcattgaccaaaacatcatatcagcattttttcttctaagattttgggagcaccaaagggatacactaacaggatatactctttataatgggtttggagaactgtctgcagctacttcttttaaaaaggtgatctacacagtagaaattagacaagtttggtaatgagatctgcaatccaaataaaataaattcattgctaacctttttcttttcttttcaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttga

SEQ ID NO: 67 - DNA, 4654 bp, Homo sapiens
ggttagtgatgaggctagtgatgaggctgtgtgcttctgagctgggcatccgaaggcatccttggggaagctgagggcacgaggaggggctgccagactccgggagctgctgcctggctgggattcctacacaatgcgttgcctggctccacgccctgctgggtcctacctgtcagagccccaaggcagctcacagtgtgccaccatggagttggggcccctagaaggtggctacctggagcttcttaacagcgatgctgaccccctgtgcctctaccacttctatgaccagatggacctggctggagaagaagagattgagctctactcagaacccgacacagacaccatcaactgcgaccagttcagcaggctgttgtgtgacatggaaggtgatgaagagaccagggaggcttatgccaatatcgcggaactggaccagtatgtcttccaggactcccagctggagggcctgagcaaggacattttcaagcacataggaccagatgaagtgatcggtgagagtatggagatgccagcagaagttgggcagaaaagtcagaaaagacccttcccagaggagcttccggcagacctgaagcactggaagccagctgagccccccactgtggtgactggcagtctcctagtgggaccagtgagcgactgctccaccctgccctgcctgccactgcctgcgctgttcaaccaggagccagcctccggccagatgcgcctggagaaaaccgaccagattcccatgcctttctccagttcctcgttgagctgcctgaatctccctgagggacccatccagtttgtccccaccatctccactctgccccatgggctctggcaaatctctgaggctggaacaggggtctccagtatattcatctaccatggtgaggtgccccaggccagccaagtaccccctcccagtggattcactgtccacggcctcccaacatctccagaccggccaggctccaccagccccttcgctccatcagccactgacctgcccagcatgcctgaacctgccctgacctcccgagcaaacatgacagagcacaagacgtcccccacccaatgcccggcagctggagaggtctccaacaagcttccaaaatggcctgagccggtggagcagttctaccgctcactgcaggacacgtatggtgccgagcccgcaggcccggatggcatcctagtggaggtggatctggtgcaggccaggctggagaggagcagcagcaagagcctggagcgggaactggccaccccggactgggcagaacggcagctggcccaaggaggcctggctgaggtgctgttggctgccaaggagcaccggcggccgcgtgagacacgagtgattgctgtgctgggcaaagctggtcagggcaagagctattgggctggggcagtgagccgggcctgggcttgtggccggcttccccagtacgactttgtcttctctgtcccctgccattgcttgaaccgtccgggggatgcctatggcctgcaggatctgctcttctccctgggcccacagccactcgtggcggccgatgaggttttcagccacatcttgaagagacctgaccgcgttctgctcatcctagacggcttcgaggagctggaagcgcaagatggcttcctgcacagcacgtgcggaccggcaccggcggagccctgctccctccgggggctgctggccggccttttccagaagaagctgctccgaggttgcaccctcctcctcacagcccggccccggggccgcctggtccagagcctgagcaaggccgacgccctatttgagctgtccggcttctccatggagcaggcccaggcatacgtgatgcgctactttgagagctcagggatgacagagcaccaagacagagccctgacgctcctccgggaccggccacttcttctcagtcacagccacagccctactttgtgccgggcagtgtgccagctctcagaggccctgctggagcttggggaggacgccaagctgccctccacgctcacgggactctatgtcggcctgctgggccgtgcagccctcgacagcccccccggggccctggcagagctggccaagctggcctgggagctgggccgcagacatcaaagtaccctacaggaggaccagttcccatccgcagacgtgaggacctgggcgatggccaaaggcttagtccaacacccaccgcgggccgcagagtccgagctggccttccccagcttcctcctgcaatgcttcctgggggccctgtggctggctctgagtggcgaaatcaaggacaaggagctcccgcagtacctagcattgaccccaaggaagaagaggccctatgacaactggctggagggcgtgccacgctttctggctgggctgatcttccagcctcccgcccgctgcctgggagccctactcgggccatcggcggctgcctcggtggacaggaagcagaaggtgcttgcgaggtacctgaagcggctgcagccggggacactgcgggcgcggcagctgctggagctgctgcactgcgcccacgaggccgaggaggctggaatttggcagcacgtggtacaggagctccccggccgcctctcttttctgggcacccgcctcacgcctcctgatgcacatgtactgggcaaggccttggaggcggcgggccaagacttctccctggacctccgcagcactggcatttgcccctctggattggggagcctcgtgggactcagctgtgtcacccgtttcagggctgccttgagcgacacggtggcgctgtgggagtccctgcagcagcatggggagaccaagctacttcaggcagcagaggagaagttcaccatcgagcctttcaaagccaagtccctgaaggatgtggaagacctgggaaagcttgtgcagactcagaggacgagaagttcctcggaagacacagctggggagctccctgctgttcgggacctaaagaaactggagtttgcgctgggccctgtctcaggcccccaggctttccccaaactggtgcggatcctcacggccttttcctccctgcagcatctggacctggatgcgctgagtgagaacaagatcggggacgagggtgtctcgcagctctcagccaccttcccccagctgaagtccttggaaaccctcaatctgtcccagaacaacatcactgacctgggtgcctacaaactcgccgaggccctgccttcgctcgctgcatccctgctcaggctaagcttgtacaataactgcatctgcgacgtgggagccgagagcttggctcgtgtgcttccggacatggtgtccctccgggtgatggacgtccagtacaacaagttcacggctgccggggcccagcagctcgctgccagccttcggaggtgtcctcatgtggagacgctggcgatgtggacgcccaccatcccattcagtgtccaggaacacctgcaacaacaggattcacggatcagcctgagatgatcccagctgtgctctggacaggcatgttctctgaggacactaaccacgctggaccttgaactgggtacttgtggacacagctcttctccaggctgtatcccatgagcctcagcatcctggcacccggcccctgctggttcagggttggcccctgcccggctgcggaatgaaccacatcttgctctgctgacagacacaggcccggctccaggctcctttagcgcccagttgggtggatgcctggtggcagctgcggtccacccaggagccccgaggccttctctgaaggacattgcggacagccacggccaggccagagggagtgacagaggcagccccattctgcctgcccaggcccctgccaccctggggagaaagtacttctttttttttatttttagacagagtctcactgttgcccaggctggcgtgcagtggtgcgatctgggttcactgcaacctccgcctcttgggttcaagcgattcttctgcttcagcctcccgagtagctgggactacaggcacccaccatcatgtctggctaatttttcatttttagtagagacagggttttgccatgttggccaggctggtctcaaactcttgacctcaggtgatccacccacctcagcctcccaaagtgctgggattacaagcgtgagccactgcaccgggccacagagaaagtacttctccaccctgctctccgaccagacaccttgacagggcacaccgggcactcagaagacactgatgggcaacccccagcctgctaattccccagattgcaacaggctgggcttcagtggcagctgcttttgtctatgggactcaatgcactgacattgttggccaaagccaaagctaggcctggccagatgcaccagcccttagcagggaaacagctaatgggacactaatggggcggtgagaggggaacagactggaagcacagcttcatttcctgtgtcttttttcactacattataaatgtctctttaatgtcacaggcaggtccagggtttgagttcataccctgttaccattttggggtacccactgctctggttatctaatatgtaacaagccaccccaaatcatagtggcttaaaacaacactcacattta

SEQ ID NO: 68 - RNA, 20 nt, Artificial Sequence
  synthetic primer
cgcgagcacagcuaaggcca

SEQ ID NO: 69 - RNA, 20 nt, Artificial Sequence
  synthetic primer
gauauuggcauaagccuccc

SEQ ID NO: 70 - DNA, 996 bp, Artificial Sequence
  sr39TK cDNA sequence codon optimized
atgcctacactgctgcgggtgtacatcgatggccctcacggcatgggcaagaccacaaccacacagctgctggtggccctgggcagcagggacgatatcgtgtacgtgccagagcccatgacatattggcgcgtgctgggagcatccgagacaatcgccaacatctacaccacacagcacagactggatcagggagagatctccgccggcgacgcagcagtggtcatgaccagcgcccagatcacaatgggcatgccatatgcagtgaccgacgccgtgctggcacctcacatcggaggagaggcaggctctagccacgcaccaccccctgccctgacaatctttctggatcggcaccctatcgccttcatgctgtgctacccagccgccagatatctgatgggcagcatgaccccacaggccgtgctggccttcgtggccctgatcccacccaccctgccaggaacaaatatcgtgctgggcgccctgccagaggacaggcacatcgatagactggccaagaggcagcgccccggagagcggctggacctggcaatgctggcagcaatcaggagagtgtacggcctgctggccaacaccgtgcggtatctgcagtgtggaggctcctggagagaggactggggacagctgtctggaacagcagtgcctccacagggagcagagccacagtccaatgcaggacctaggccacacatcggcgataccctgttcacactgtttcgcgcaccagagctgctggcacctaacggcgatctgtacaacgtgttcgcatgggcactggacgtgctggcaaagcggctgagatctatgcacgtgttcatcctggactacgaccagagcccagccggctgtagagatgccctgctgcagctgacaagcggcatggtgcagacccacgtgaccacacccggctctattccaacaatctgcgacctggctaggacctttgcaagagaaatgggcgaagctaactga

SEQ ID NO: 71 - protein, 331 aa, Artificial Sequence
  sr39TK amino acid sequence
MPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTIFLDRHPIAFMLCYPAARYLMGSMTPQAVLAFVALIPPTLPGTNIVLGALPEDRHIDRLAKRQRPGERLDLAMLAAIRRVYGLLANTVRYLQCGGSWREDWGQLSGTAVPPQGAEPQSNAGPRPHIGDTLFTLFRAPELLAPNGDLYNVFAWALDVLAKRLRSMHVFILDYDQSPAGCRDALLQLTSGMVQTHVTTPGSIPTICDLARTFAREMGEAN

SEQ ID NO: 72 - DNA, 1508 bp, Artificial Sequence
  Human T cell receptor alpha chain (TRAC) mRNA sequence
ttttgaaacccttcaaaggcagagacttgtccagcctaacctgcctgctgctcctagctcctgaggctcagggcccttggcttctgtccgctctgctcagggccctccagcgtggccactgctcagccatgctcctgctgctcgtcccagtgctcgaggtgatttttaccctgggaggaaccagagcccagtcggtgacccagcttggcagccacgtctctgtctctgaaggagccctggttctgctgaggtgcaactactcatcgtctgttccaccatatctcttctggtatgtgcaataccccaaccaaggactccagcttctcctgaagtacacatcagcggccaccctggttaaaggcatcaacggttttgaggctgaatttaagaagagtgaaacctccttccacctgacgaaaccctcagcccatatgagcgacgcggctgagtacttctgtgctgtgagtgatctcgaaccgaacagcagtgcttccaagataatctttggatcagggaccagactcagcatccggccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgagatctgcaagattgtaagacagcctgtgctccctcgctccttcctctgcattgcccctcttctccctctccaaacagagggaactctcctacccccaaggaggtgaaagctgctaccacctctgtgcccccccggtaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctggagaatgttgtgaggggtttatttttttttaatagtgttcataaagaaatacatagtattcttcttctcaagacgtggggggaaattatctcattatcgaggccctgctatgctgtgtgtctgggcgtgttgtatgtcctgctgccgatgccttcattaaaatgatttggaa

SEQ ID NO: 73 - DNA, 2042 bp, Artificial Sequence
  Human T cell receptor beta chain (TRBC1) mRNA sequence
tgcatcctagggacagcatagaaaggaggggcaaagtggagagagagcaacagacactgggatggtgaccccaaaacaatgagggcctagaatgacatagttgtgcttcattacggcccattcccagggctctctctcacacacacagagcccctaccagaaccagacagctctcagagcaaccctggctccaacccctcttccctttccagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcagacaagactagatccaaaaagaaaggaaccagcgcacaccatgaaggagaattgggcacctgtggttcattcttctcccagattctcagcccaacagagccaagcagctgggtcccctttctatgtggcctgtgtaactctcatctgggtggtgccccccatccccctcagtgctgccacatgccatggattgcaaggacaatgtggctgacatctgcatggcagaagaaaggaggtgctgggctgtcagaggaagctggtctgggcctgggagtctgtgccaactgcaaatctgactttacttttaattgcctatgaaaataaggtctctcatttattttcctctccctgctttctttcagactgtggctttacctcgggtaagtaagcccttccttttcctctccctctctcatggttcttgacctagaaccaaggcatgaagaactcacagacactggagggtggagggtgggagagaccagagctacctgtgcacaggtacccacctgtccttcctccgtgccaacagtgtcctaccagcaaggggtcctgtctgccaccatcctctatgagatcctgctagggaaggccaccctgtatgctgtgctggtcagcgcccttgtgttgatggccatggtaagcaggagggcaggatggggccagcaggctggaggtgacacactgacaccaagcacccagaagtatagagtccctgccaggattggagctgggcagtagggagggaagagatttcattcaggtgcctcagaagataacttgcacctctgtaggatcacagtggaagggtcatgctgggaaggagaagctggagtcaccagaaaacccaatggatgttgtgatgagccttactatttgtgtggtcaatgggccctactactttctctcaatcctcacaactcctggctcttaataacccccaaaactttctcttctgcaggtcaagagaaaggatttctgaaggcagccctggaagtggagttaggagcttctaacccgtcatggtttcaatacacattcttcttttgccagcgcttctgaagagctgctctcacctctctgcatcccaatagatatccccctatgtgcatgcacacctgcacactcacggctgaaatctccctaacccagggggaccttagcatgcctaagtgactaaaccaataaaaatgttctggtctggcctgactctgacttgtgaatgtctggatagctccttggctgtctctgaactccctgtgactctccccattcagtcaggatagaaacaagaggtattcaaggaaaatgcagactcttcacgtaagagggatgaggggcccaccttgagatcaatagcagaa

SEQ ID NO: 74 - DNA, 2008 bp, Artificial Sequence
  Human TRBC2 T cell receptor beta constant 2 (TCRB2) sequence
atggcgtagtccccaaagaacgaggacctagtaacataattgtgcttcattatggtcctttcccggccttctctctcacacatacacagagcccctaccaggaccagacagctctcagagcaaccctagccccattacctcttccctttccagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcaggtgagtggggcctggggagatgcctggaggagattaggtgagaccagctaccagggaaaatggaaagatccaggtagcggacaagactagatccagaagaaagccagagtggacaaggtgggatgatcaaggttcacagggtcagcaaagcacggtgtgcacttcccccaccaagaagcatagaggctgaatggagcacctcaagctcattcttccttcagatcctgacaccttagagctaagctttcaagtctccctgaggaccagccatacagctcagcatctgagtggtgtgcatcccattctcttctggggtcctggtttcctaagatcatagtgaccacttcgctggcactggagcagcatgagggagacagaaccagggctatcaaaggaggctgactttgtactatctgatatgcatgtgtttgtggcctgtgagtctgtgatgtaaggctcaatgtccttacaaagcagcattctctcatccatttttcttcccctgttttctttcagactgtggcttcacctccggtaagtgagtctctcctttttctctctatctttcgccgtctctgctctcgaaccagggcatggagaatccacggacacaggggcgtgagggaggccagagccacctgtgcacaggtacctacatgctctgttcttgtcaacagagtcttaccagcaaggggtcctgtctgccaccatcctctatgagatcttgctagggaaggccaccttgtatgccgtgctggtcagtgccctcgtgctgatggccatggtaaggaggagggtgggatagggcagatgatgggggcaggggatggaacatcacacatgggcataaaggaatctcagagccagagcacagcctaatatatcctatcacctcaatgaaaccataatgaagccagactggggagaaaatgcagggaatatcacagaatgcatcatgggaggatggagacaaccagcgagccctactcaaattaggcctcagagcccgcctcccctgccctactcctgctgtgccatagcccctgaaaccctgaaaatgttctctcttccacaggtcaagagaaaggattccagaggctagctccaaaaccatcccaggtcattcttcatcctcacccaggattctcctgtacctgctcccaatctgtgttcctaaaagtgattctcactctgcttctcatctcctacttacatgaatacttctctcttttttctgtttccctgaagattgagctcccaacccccaagtacgaaataggctaaaccaataaaaaattgtgtgttgggcctggttgcatttcaggagtgtctgtggagttctgctcatcactgacctatcttctgatttagggaaagcagcattcgcttggacatctgaagtgacagccctctttctctccacccaatgctgctttctcctgttcatcctgatggaagtctcaacaca

(19) United States
(12) Patent Application Publication
(10) Pub. No.: US 2021/0123022 A1
(43) Pub. Date: Apr. 29, 2021

(54) STEM CELL-ENGINEERED INKT CELL-BASED OFF-THE-SHELF CELLULAR THERAPY

(71) Applicant: The Regents of the University of California, Oakland, CA (US)

(72) Inventors: Lili Yang, Los Angeles, CA (US); Yanni Zhu, Los Angeles, CA (US); Christopher Seet, Los Angeles, CA (US); Amelie Montel Hagen, Los Angeles, CA (US); Gay Crooks, Los Angeles, CA (US)

(73) Assignee: The Regents of the University of California, Oakland, CA (US)

(21) Appl. No.: 17/251,118

(22) PCT Filed: Jun. 12, 2019
(86) PCT No.: PCT/US19/36786

§ 371 Date: Dec. 10, 2020

Related U.S. Application Data
(60) Provisional application No. 62/683,750, filed on Jun. 12, 2018.

(51) Int. Cl. C12N 5/0783; C07K 14/725; C07K 14/74; C12N 15/86; A61K 35/17

(52) U.S. Cl. CPC C12N 5/0646; C07K 14/7051; C07K 14/70539; C12N 2502/1185; A61K 35/17; C12N 2740/15043; C12N 15/86

(57) ABSTRACT

Embodiments of the disclosure include compositions and methods related to engineered invariant natural killer T (iNKT) cells for off-the-shelf use for clinical therapy. In particular embodiments, the iNKT cells are produced from hematopoietic stem progenitor cells and also are suitable for allogeneic cellular therapy because they are HLA negative. In specific embodiments, the cells are cultured in a particular in vitro three-dimensional artificial thymic organoid system and the cells have imaging and suicide targeting capabilities.

### CROSS-REFERENCE TO RELATED APPLICATIONS

This application claims the benefit of U.S. Provisional Applications No. 62/683,750, filed Jun. 12, 2018, the contents of which is incorporated into the present application by reference in its entirety.

### TECHNICAL FIELD

Embodiments of the disclosure concern at least the fields of immunology, cell biology, molecular biology, and medicine, including at least cancer medicine.

### BACKGROUND

Cancer affects tens of millions of people worldwide and is a leading threat to public health in the United States and in the state of California. It is the second leading cause of death in California, resulting in more than 56,000 deaths each year, and also brings devastating economic impacts to the state. Despite the existing therapies, cancer patients still suffer from the ineffectiveness of these treatments, their toxicities, and the risk of relapse. Novel therapies for cancer are therefore in desperately needed. Over the past decade, immunotherapy has become the new-generation cancer medicine. In particular, cell-based cellular therapies have shown great promise. An outstanding example is the chimeric antigen receptor (CAR)-engineered adoptive T cells therapy, which targets certain blood cancers at impressive efficacy.

However, most of the current protocols for treatment consist of autologous adoptive cell transfer, wherein immune cells collected from a patient are manufactured and used to treat this single patient. Such an approach is costly, manufacture labor intensive, and difficult to broadly deliver to all patients in need. Allogenic immune cellular products that can be manufactured at a large-scale and can be readily distributed to treat a higher number of patients therefore are in great demand.

Despite existing therapies, cancer patients still suffer from the ineffectiveness of these treatments, their toxicities, and the risk of relapse. Novel therapies for diseases, such as cancer and autoimmune diseases, are therefore in desperate demand. The present disclosure provides solutions to a long-felt need for therapies, but also therapies that can be delivered or distributed more widely.

### BRIEF SUMMARY

Embodiments are provided to address the need for new therapies, more particularly, the need for cellular therapies that are not hampered by the challenges posed for individualizing therapy using autologous cells. The ability to manufacture a therapeutic cell population or a cell population that can be used to create a therapeutic cell population “off-the-shelf” increases the availability and usefulness of new cellular therapies.

Embodiments concern an engineered invariant natural killer T (iNKT) cell or a population of engineered iNKT cells. In at least some cases, the engineered iNKT cells comprise an engineered chimeric antigen receptor (CAR; CAR-iNKT cells) and/or engineered T cell receptor (TCR-iNKT cells). Any embodiment discussed in the context of a cell can be applied to a population of such cells. In particular embodiments, an engineered iNKT cell comprises a nucleic acid comprising 1, 2, and/or 3 of the following: i) all or part of an invariant alpha T-cell receptor coding sequence; ii) all or part of an invariant beta T-cell receptor coding sequence, or iii) a suicide gene. In further embodiments, there is an engineered iNKT cell comprising a nucleic acid having a sequence encoding: i) all or part of an invariant alpha T-cell receptor; ii) all or part of an invariant beta T-cell receptor, and/or iii) a suicide gene product.

Further aspects relate to engineered iNKT cells with increased levels of NK activation receptors, decreased levels of NK inhibitory receptors, and/or increased levels of cytotoxic molecules. In some embodiments, the NK activation receptors comprises NKG2D and/or DNAM-1. In some embodiments, cytotoxic molecules comprise Perforin and/or Granzyme B. In some embodiments, the inhibitor receptors comprise KIR. The increase or decrease may be with respect to the levels of the same marker in non-engineered iNKTs isolated from a healthy individual. Further aspects relate to a population of engineered iNKT cells, wherein the population of cells has increased levels of NK activation receptors, decreased levels of NK inhibitory receptors, and/or increased levels of cytotoxic molecules. In some embodiments, the population of engineered iNKT cells has at least, exactly, or greater than 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of cells that express high levels of NKG2D. In some embodiments, the population of engineered iNKT cells has at least, exactly, or greater than 80, 81, 82, 83, 84, 85, 86, 87, 88, 89, 90, 91, 92, 93, 94, 95, 96, 97, 98, or 99% of cells that express high levels of DNAM-1. In some embodiments, the population of engineered iNKT cells has at most, exactly, or less than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30% of cells that express high levels of KIR. In some embodiments, the population of engineered iNKT cells has at least, exactly, or greater than 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of cells that express high levels of Perforin. In some embodiments, the population of engineered iNKT cells has at least, exactly, or greater than 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of cells that express high levels of Granzyme B. Further aspects of the disclosure relate to an engineered iNKT cell or population of cells comprising high levels of NK activators NKG2D and DNAM-1, low or undetectable level of the NK inhibitory receptor KIR, and high levels of cytotoxic molecules Perforin and Granzyme B. Further aspects of the disclosure relate to a population of engineered iNKT cells, wherein greater than 90% of the population comprises high levels of NK activators NKG2D and DNAM-1, a low or undetectable level of the NK inhibitory receptor KIR, and high levels of cytotoxic molecules Perforin and Granzyme B.

In some embodiments, the engineered iNKT cell comprises a nucleic acid under the control of a heterologous promoter, which means the promoter is not the same genomic promoter that controls the transcription of the nucleic acid. It is contemplated that the engineered iNKT cell comprises an exogenous nucleic acid comprising one or more coding sequences, some or all of which are under the control of a heterologous promoter in many embodiments described herein.

It is specifically noted that any embodiment discussed in the context of a particular cell or cell population embodiment may be employed with respect to any other cell or cell population embodiment. Moreover, any embodiment employed in the context of a specific method may be implemented in the context of any other methods described herein. Furthermore, aspects of different methods described herein may be combined so as to achieve other methods, as well as to create or describe the use of any cells or cell populations. It is specifically contemplated that aspects of one or more embodiments may be combined with aspects of one or more other embodiments described herein. Furthermore, any method described herein may be phrased to set forth one or more uses of cells or cell populations described herein. For instance, use of engineered iNKT cells or an iNKT cell population can be set forth from any method described herein.

In a particular embodiment, there is an engineered invariant natural killer T (iNKT) cell that expresses at least one invariant natural killer T-cell receptor (iNKT TCR) and an exogenous suicide gene product, wherein the at least one iNKT TCR is expressed from an exogenous nucleic acid and/or from an endogenous invariant TCR gene that is under the transcriptional control of a recombinantly modified promoter region. An iNKT TCR refers to a “TCR that recognizes lipid antigen presented by a CD1d molecule.” It may include an alpha-TCR, a beta-TCR, or both. In some cases, the TCR utilized can belong to a broader group of “invariant TCR”, such as a MAIT cell TCR, GEM cell TCR, or gamma/delta TCR, resulting in HSC-engineered MAIT cells, GEM cells, or gamma/delta T cells, respectively.

In certain embodiments, there are engineered iNKT cell populations. In a particular embodiment, there is an engineered iNKT cell population comprising: engineered iNKT clonal cells comprising either an altered genomic invariant T-cell receptor sequence or an exogenous nucleic acid encoding an invariant T-cell receptor (TCR) and lacking expression of one or more HLA-I or HLA-II genes. An “altered genomic invariant T-cell receptor sequence” means a sequence that has been altered by recombinant DNA technology. The term “clonal” cells refers to iNKT cells engineered to express a clonal transgenic iNKT TCR. In some embodiments, the clonal cells are from the same progenitor cell. It is contemplated that in some embodiments, there is a population of mixed clonal cells meaning the population comprises clonal cells that are from a set of progenitor cells; the set may be, be at least or be at most 10, 20, 30, 40, 50, 60 70, 80, 90, 100, 200, 300, 400, 500, 600, 700, 800, 900, 1000 or more progenitor cells (or any range derivable therein) meaning the cells in the population are progeny of the set of progenitor cells initially transfected/infected. In cases of cells comprising an exogenous nucleic acid or an altered genomic DNA sequence clonal cells may arise from an ancestor cell in which the exogenous nucleic acid was introduced. Some embodiments concern a population of clonal cells, meaning the population comprises progeny cells that arose from the same ancestor cell. It is contemplated that some populations of cells may contain a mix of different clonal cells, meaning the population arose from different ancestor cells that contain an exogenous nucleic acid but that may differ in a discernable way, such as the integration site for the exogenous nucleic acid. A nucleic acid sequence that has been introduced into a cell (alone or as part of a longer nucleic acid sequence) and becomes integrated such that progeny cells contain the integrated nucleic acid sequence is considered an exogenous nucleic acid. An introduced nucleic acid sequence that is maintained extrachromosomally is also considered an exogenous nucleic acid.

In embodiments where part of an iNKT alpha T-cell receptor or part of an iNKT beta T-cell receptor are utilized, it is contemplated that embodiments involve a functional part of an iNKT alpha T-cell receptor or a functional part of an iNKT beta T-cell receptor such that the cell expressing both of them is a functional iNKT cell at least based on an assay that evaluates the ability to recognize lipid antigen presented by a CD1d molecule.

In some embodiments, a nucleic acid comprises a sequence that is, is at least, or is at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% identical (or any range derivable therein) to a sequence encoding 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 amino acids or contiguous amino acid residues of an iNKT TCR-alpha or iNKT TCR-beta polypeptide (or any range derivable therein).

In certain embodiments, a suicide gene is enzyme-based, meaning the gene product of the suicide gene is an enzyme and the suicide function depends on enzymatic activity. One or more suicide genes may be utilized in a single cell or clonal population. In some embodiments, the suicide gene encodes herpes simplex virus thymidine kinase (HSV-TK), purine nucleoside phosphorylase (PNP), cytosine deaminase (CD), carboxypetidase G2, cytochrome P450, linamarase, beta-lactamase, nitroreductase (NTR), carboxypeptidase A, or inducible caspase 9. Methods in the art for suicide gene usage may be employed, such as in U.S. Pat. No. 8,628,767, U.S. Patent Application Publication 20140369979, U.S. 20140242033, and U.S. 20040014191, all of which are incorporated by reference in their entirety. In further embodiments, a TK gene is a viral TK gene, .i.e., a TK gene from a virus. In particular embodiments, the TK gene is a herpes simplex virus TK gene. In some embodiments, the suicide gene product is activated by a substrate. Thymidine kinase is a suicide gene product that is activated by ganciclovir, penciclovir, or a derivative thereof. In certain embodiments, the substrate activating the suicide gene product is labeled in order to be detected. In some instances, the substrate that may be labeled for imaging. In some embodiments, the suicide gene product may be encoded by the same or a different nucleic acid molecule encoding one or both of TCR-alpha or TCR-beta. In certain embodiments, the suicide gene is sr39TK or inducible caspase 9. In alternative embodiments, the cell does not express an exogenous suicide gene. In some embodiments, the engineered iNKT cell specifically binds to alpha-galactosylceramide (α-GC).

In additional embodiments, a cell is lacking or has reduced surface expression of at least one HLA-I or HLA-II molecule. In some embodiments, the lack of surface expression of HLA-I and/or HLA-II molecules is achieved by disrupting the genes encoding individual HLA-VII molecules, or by disrupting the gene encoding B2M (beta 2 microglobulin) that is a common component of all HLA-I complex molecules, or by discrupting the genes encoding CIITA (the class II major histocompatibility complex transactivator) that is a critical transcription factor controlling the expression of all HLA-II genes. In specific embodiments, the cell lacks the surface expression of one or more HLA-I and/or HLA-II molecules, or expresses reduced levels of such molecules by (or by at least) 50, 60, 70, 80, 90, 100% (or any range derivable therein). In some embodiments, the HLA-I or HLA-II are not expressed in the iNKT cell because the cell was manipulated by gene editing. In some embodiments, the gene editing involved is CRISPR-Cas9. Instead of Cas9, CasX or CasY may be involved. Zinc finger nuclease (ZFN) and TALEN are other gene editing technologies, as well as Cpfl, all of which may be employed. In other embodiments, the iNKT cell comprises one or more different siRNA or miRNA molecules targeted to reduce expression of HLA-I/II molecules, B2M, and/or CIITA.

In some embodiments, an iNKT cell comprises a recombinant vector or a nucleic acid sequence from a recombinant vector that was introduced into the cells. In certain embodiments the recombinant vector is or was a viral vector. In further embodiments, the viral vector is or was a lentivirus, a retrovirus, an adeno-associated virus (AAV), a herpesvirus, or adenovirus. It is understood that the nucleic acid of certain viral vectors integrate into the host genome sequence.

In some embodiments, a cell was not exposed to media comprising animal serum. In further embodiments, a cell is or was frozen. In some embodiments, the cell has previously been frozen and the previously frozen cell is stable at room temperature for at least one hour.

In some embodiments, the cell has previously been frozen and the previously frozen cell is stable at room temperature for at least 1, 2, 3, 4, 5, 6, 7, 8, 10, 15, 20, 24, 30, or 48 hours (or any derivable range therein). In certain embodiments, a cell or a population of cells in a solution comprises dextrose, one or more electrolytes, albumin, dextran, and/or DMSO. In a further embodiments, the cell is in a solution that is sterile, nonpyogenic, and isotonic.

In certain embodiments, an iNKT cell has been or is activated. In specific embodiments, the iNKT cells have been activated with alpha-galactosylceramide (α-GC).

In embodiments involving multiple cells, a cell population may comprise, comprise at least, or comprise at most about 10², 10³, 10^{4,}, 10⁵, 10⁶, 10^{7,}, 10⁸, 10⁹, 10¹⁰, 10¹¹, 10¹², 10¹³, 10¹⁴, 10¹⁵ cells or more (or any range derivable therein), which are engineered iNKT cells in some embodiments. In some cases, a cell population comprises at least about 10⁶-10¹² engineered iNKT cells. It is contemplated that in some embodiments, that a population of cells with these numbers is produced from a single batch of cells and are not the result of pooling batches of cells separately produced.

In specific embodiments, there is an iNKT cell population comprising: clonal iNKT cells comprising one or more exogenous nucleic acids encoding an iNKT T-cell receptor (TCR) and a thymidine kinase suicide gene product, wherein the clonal iNKT cells have been engineered not to express functional beta-2-microglobulin (B2M), and/or class II, major histocompatibility complex, or transactivator (CIITA) and wherein the cell population is at least about 10⁶-10¹² total cells and comprises at least about 10²-10⁶ engineered iNKT cells. In certain instances, the cells are frozen in a solution.

A number of embodiments concern methods of preparing an iNKT cell or a population of cells, particularly a population in which some are all the cells are clonal. In certain embodiments, a cell population comprises cells in which at least or at most 50%, 55%, 60%, 65%, 70%, 75%, 80%, 85%, 90%, 95%, 96%, 97%, 98%, 99%, 100% (or any range derivable therein) of the cells are clonal, i.e. , the percentage of cells that have been derived from the same ancestor cell as another cell in the population. In other embodiments, a cell population comprises a cell population that is comprised of cells arising from, from at least, or from at most 1, 2, 3, 4, 5, 6, 7, 8, 9, 10, 11, 12, 13, 14, 15, 16, 17, 18, 19, 20, 21, 22, 23, 24, 25, 26, 7, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 (or any range derivable therein) different parental cells.

Methods for preparing, making, manufacturing, and using engineered iNKT cells and iNKT cell populations are provided. Methods include 1, 2, 3, 4, 5, 6, 7, 8, 9, 10, 11, 12, 13, 14, 15 or more of the following steps in embodiments: obtaining hematopoietic cells; obtaining hematopoietic progenitor cells; obtaining progenitor cells capable of becoming one or more hematopoietic cells; obtaining progenitor cells capable of becoming iNKT cells; selecting cells from a population of mixed cells using one or more cell surface markers; selecting CD34+ cells from a population of cells; isolating CD34+ cells from a population of cells; separating CD34+ and CD34− cells from each other; selecting cells based on a cell surface marker other than or in addition to CD34; introducing into cells one or more nucleic acids encoding an iNKT T-cell receptor (TCR); infecting cells with a viral vector encoding an iNKT T-cell receptor (TCR); transfecting cells with one or more nucleic acids encoding an iNKT T-cell receptor (TCR); transfecting cells with an expression construct encoding an iNKT T-cell receptor (TCR); integrating an exogenous nucleic acid encoding an iNKT T-cell receptor (TCR) into the genome of a cell; introducing into cells one or more nucleic acids encoding a suicide gene product; infecting cells with a viral vector encoding a suicide gene product; transfecting cells with one or more nucleic acids encoding a suicide gene product; transfecting cells with an expression construct encoding a suicide gene product; integrating an exogenous nucleic acid encoding a suicide gene product into the genome of a cell; introducing into cells one or more nucleic acids encoding one or more polypeptides and/or nucleic acid molecules for gene editing; infecting cells with a viral vector encoding one or more polypeptides and/or nucleic acid molecules for gene editing; transfecting cells with one or more nucleic acids encoding one or more polypeptides and/or nucleic acid molecules for gene editing; transfecting cells with an expression construct encoding one or more polypeptides and/or nucleic acid molecules for gene editing; integrating an exogenous nucleic acid encoding one or more polypeptides and/or nucleic acid molecules for gene editing; editing the genome of a cell; editing the promoter region of a cell; editing the promoter and/or enhancer region for an iNKT TCR gene; eliminating the expression one or more genes; eliminating expression of one or more HLA-I/II genes in the isolated human CD34+ cells; transfecting into a cell one or more nucleic acids for gene editing; culturing isolated or selected cells; expanding isolated or selected cells; culturing cells selected for one or more cell surface markers; culturing isolated CD34+ cells expressing iNKT TCR; expanding isolated CD34+ cells; culturing cells under conditions to produce or expand iNKT cells; culturing cells in an artificial thymic organoid (ATO) system to produce iNKT cells; culturing cells in serum-free medium; culturing cells in an ATO system, wherein the ATO system comprises a 3D cell aggregate comprising a selected population of stromal cells that express a Notch ligand and a serum-free medium. It is specifically contemplated that one or more steps may be excluded in an embodiment.

In some embodiments, there are methods of preparing a population of clonal iNKT cells comprising: a) selecting CD34+ cells from human peripheral blood cells (PBMCs); b) introducing one or more nucleic acids encoding a human T-cell receptor (TCR); c) eliminating surface expression of one or more HLA-I/II genes in the isolated human CD34+ cells; and, d) culturing isolated CD34+ cells expressing iNKT TCR in an artificial thymic organoid (ATO) system to produce iNKT cells, wherein the ATO system comprises a 3D cell aggregate comprising a selected population of stromal cells that express a Notch ligand and a serum-free medium.

Cells that may be used to create engineered iNKT cells are hematopoietic progenitor stem cells. Cells may be from peripheral blood mononuclear cells (PBMCs), bone marrow cells, fetal liver cells, embryonic stem cells, cord blood cells, induced pluripotent stem cells (iPS cells), or a combination thereof.

In some embodiments, methods comprise isolating CD34− cells or separating CD34− and CD34+ cells. While embodiments involve manipulating the CD34+ cells further, CD34− cells may be used in the creation of iNKT cells. Therefore, in some embodiments, the CD34− cells are subsequently used, and may be saved for this purpose.

Certain methods involve culturing selected CD34+ cells in media prior to introducing one or more nucleic acids into the cells. Culturing the cells can include incubating the selected CD34+ cells with media comprising one or more growth factors. In some embodiments, one or more growth factors comprise c-kit ligand, flt-3 ligand, and/or human thrombopoietin (TPO). In further embodiments, the media includes c-kit ligand, flt-3 ligand, and TPO. In some embodiments, the concentration of the one or more growth factors is between about 5 ng/ml to about 500 ng/ml with respect to either each growth factor or the total of any and all of these particular growth factors. The concentration of a single growth factor or the combination of growth factors in media can be about, at least about, or at most about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500 (or any range derivable) ng/ml or μg/ml or more.

In some embodiments, a nucleic acid may comprise a nucleic acid sequence encoding an α-TCR and/or a β-TCR, as discussed herein. In certain embodiments, one nucleic acid encodes both the α-TCR and the β-TCR. In additional embodiments, a nucleic acid further comprises a nucleic acid sequence encoding a suicide gene product. In some embodiments, a nucleic acid molecule that is introduced into a selected CD34+ cell encodes the α-TCR, the β-TCR, and the suicide gene product. In other embodiments, a method also involves introducing into the selected CD34+ cells a nucleic acid encoding a suicide gene product, in which case a different nucleic acid molecule encodes the suicide gene product than a nucleic acid encoding at least one of the TCR genes.

As discussed, in some embodiments the iNKT cells do not express the HLA-I and/or HLA-II molecules on the cell surface, which may be achieved by discrupting the expression of genes encoding beta-2-microglobulin (B2M), transactivator (CIITA), or HLA-I and HLA-II molecules. In certain embodiments, methods involve eliminating surface expression of one or more HLA-I/II molecules in the isolated human CD34+ cells. In particular embodiments, eliminating expression may be accomplished through gene editing of the cell's genomic DNA. Some methods include introducing CRISPR and one or more guide RNAs (gRNAs) corresponding to B2M or CIITA into the cells. In particular embodiments, CRISPR or the one or more gRNAs are transfected into the cell by electroporation or lipid-mediated transfection. Consequently, methods may involve introducing CRISPR and one or more gRNAs into a cell by transfecting the cell with nucleic acid(s) encoding CRISPR and the one or more gRNAs. A different gene editing technology may be employed in some embodiments.

Similarly, in some embodiments, one or more nucleic acids encoding the TCR receptor are introduced into the cell. This can be done by transfecting or infecting the cell with a recombinant vector, which may or may not be a viral vector as discussed herein. The exogenous nucleic acid may incorporate into the cell's genome in some embodiments.

In some embodiments, cells are cultured in cell-free medium. In certain embodiments, the serum-free medium further comprises externally added ascorbic acid. In particular embodiments, methods involve adding ascorbic acid medium. In further embodiments, the serum-free medium further comprises 1, 2, 3, 4, 5, 6, 7, 8, 9, 10, 11, 12, 13, 14, 15, or all 16 (or a range derivable therein) of the following externally added components: FLT3 ligand (FLT3L), interleukin 7 (IL-7), stem cell factor (SCF), thrombopoietin (TPO), stem cell factor (SCF), IL-2, IL-4, IL-6, IL-15, IL-21, TNF-alpha, TGF-beta, interferon-gamma, interferon-lambda, TSLP, thymopentin, pleotrophin, or midkine. In additional embodiments, the serum-free medium comprises one or more vitamins. In some cases, the serum-free medium includes 1, 2, 3, 4, 5, 6, 7, 8, 9, 10, 11, or 12 of the following vitamins (or any range derivable therein): comprise biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or a salt thereof. In certain embodiments, medium comprises or comprise at least biotin, DL alpha tocopherol acetate, DL alpha-tocopherol, vitamin A, or combinations or salts thereof. In additional embodiments, serum-free medium comprises one or more proteins. In some embodiments, serum-free medium comprises 1, 2, 3, 4, 5, 6 or more (or any range derivable therein) of the following proteins: albumin or bovine serum albumin (BSA), a fraction of BSA, catalase, insulin, transferrin, superoxide dismutase, or combinations thereof. In other embodiments, serum-free medium comprises 1, 2, 3, 4, 5, 7, 8, 9, 10, or 11 of the following compounds: corticosterone, D-Galactose, ethanolamine, glutathione, L-carnitine, linoleic acid, linolenic acid, progesterone, putrescine, sodium selenite, or triodo-I-thyronine, or combinations thereof. In further embodiments, serum-free medium comprises a B-27® supplement, xeno-free B-27® supplement, GS21TM supplement, or combinations thereof. In additional embodiments, serum-free medium comprises or further comprises amino acids, monosaccharides, and/or inorganic ions. In some aspects, serum-free medium comprises 1, 2, 3, 4, 5, 6, 7, 8, 9, 10, 11, 12, or 13 of the following amino acids: arginine, cysteine, isoleucine, leucine, lysine, methionine, glutamine, phenylalanine, threonine, tryptophan, histidine, tyrosine, or valine, or combinations thereof. In other aspects, serum-free medium comprises 1, 2, 3, 4, 5, or 6 of the following inorganic ions: sodium, potassium, calcium, magnesium, nitrogen, or phosphorus, or combinations or salts thereof. In additional aspects, serum-free medium comprises 1, 2, 3, 4, 5, 6 or 7 of the following elements: molybdenum, vanadium, iron, zinc, selenium, copper, or manganese, or combinations thereof.

In some methods, cells are cultured in an artificial thymic organoid (ATO) system. The ATO system involves a three-dimensional (3D) cell aggregate, which is an aggregate of cells. In certain embodiments, the 3D cell aggregate comprises a selected population of stromal cells that express a Notch ligand. In some embodiments, a 3D cell aggregate is created by mixing CD34+ transduced cells with the selected population of stromal cells on a physical matrix or scaffold. In further embodiments, methods comprise centrifuging the CD34+ transduced cells and stromal cells to form a cell pellet that is placed on the physical matrix or scaffold. In certain embodiments, stromal cells express a Notch ligand that is an intact, partial, or modified DLL1, DLL4, JAG1, JAG2, or a combination thereof. In further embodiments, the Notch ligand is a human Notch ligand. In other embodiments, the Notch ligand is human DLL1.

In further aspects, the ratio between stromal cells and CD34+ cells is about, at least about, or at most about 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:25, 1:30, 1:35, 1:40, 1:45, 1:50 (or any range derivable therein). In specific embodiments, the ratio between stromal cells and CD34+ cells is about 1:5 to 1:20. In particular embodiments, the stromal cells are a murine stromal cell line, a human stromal cell line, a selected population of primary stromal cells, a selected population of stromal cells differentiated from pluripotent stem cells in vitro, or a combination thereof. In certain embodiments, stroma cells are a selected population of stromal cells differentiated from hematopoietic stem or progenitor cells in vitro. Co-culturing of CD34+ cells and stromal cells may occur for about, at least about, or at most about 1, 2, 3, 4, 5, 6, 7 days and/or 1, 2, 3, 4, 5, 6, 7, 8, 9, 10, 11, 12, 13, 14, 15, 16, 17, 18, 19, 20, 21, 22, 23, 24, or more weeks (or any range derivable therein). The stromal cells are irradiated prior to co-culturing in some embodiments.

The methods of the disclosure may produce a population of cells comprising at least 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, 1×10²⁰, or 1×10²¹ (or any derivable range therein) cells that may express a marker or have a high or low level of a certain marker. The cell population number may be one that is achieved without cell sorting based on marker expression or without cell sorting based on NK marker expression or without cell sorting based on T-cell marker expression. In some embodiments, the cell population size may be one that is achieved without cell sorting based on the binding of an antigen to a heterologous targeting element, such as a CAR, TCR, BiTE, or other heterologous tumor-targeting agent. Furthermore, the population of cells achieved may be one that comprises at least 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, 1×10²⁰, or 1×10²¹ (or any derivable range therein) cells that is made within a certain time period such as a time period that is at least, at most, or exactly 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 days or 7, 8, 9, 10, 11, 12, 13, 14, 15, 16, 17, 18, 19, 20, 21, 22, 23, 24, 25, 26, 27, 28, 29 or 30 weeks (or any derivable range therein). The high or low levels of marker expression, such as NK activators, inhibitors, or cytotoxic molecules may relate to high expression as determined by FACS analysis. In some embodiments, the high levels are relative to a non-NK cell or a non-iNKT cell, or a cell that is not a T cell. In some embodiments, high levels or low levels are determined from FACS analysis.

In some embodiments, feeder cells used in methods comprise CD34− cells. These CD34− cells may be from the same population of cells selected for CD34+ cells. In additional embodiments, cells may be activated. In certain embodiments, methods comprise activating iNKT cells. In specific embodiments, iNKT cells have been activated and expanded with alpha-galactosylceramide (α-GC). Cells may be incubated or cultured with α-GC so as to activate and expand them. In some embodiments, feeder cells have been pulsed with α-GC.

In some methods, iNKT cells lacking surface expression of one or more HLA-I or -II molecules are selected. In some aspects, selecting iNKT cells lacking surface expression of HLA-I and/or HLA-II molecules protects these cells from depletion by recipient immune cells.

Cells may be used immediately or they may be stored for future use. In certain embodiments, cells that are used to create iNKT cells are frozen, while produced iNKT cells may be frozen in some embodiments. In some aspects, cells are in a solution comprising dextrose, one or more electrolytes, albumin, dextran, and DMSO. In other embodiments, cells are in a solution that is sterile, nonpyrogenic, and isotonic. In some embodiments, the engineered iNKT cell is derived from a hematopoietic stem cell. In some embodiments, the engineered iNKT cell is derived from a G-CSF mobilized CD34+ cells. In some embodiments, the cell is derived from a cell from a human patient that doesn't have cancer. In some embodiments, the cell doesn't express an endogenous TCR.

The number of cells produced by a production cycle may be about, at least about, or at most about 10², 10³, 10⁴′, 10⁵, 10⁶, 10^{7,}, 10⁸, 10⁹, 10¹⁰, 10¹¹, 10¹², 10¹³, 10¹⁴, 10¹⁵ cells or more (or any range derivable therein), which are engineered iNKT cells in some embodiments. In some cases, a cell population comprises at least about 10⁶-10¹² engineered iNKT cells. It is contemplated that in some embodiments, that a population of cells with these numbers is produced from a single batch of cells and are not the result of pooling batches of cells separately produced—i.e., from a single production cycle.

In some embodiments, a cell population is frozen and then thawed. The cell population may be used to create engineered iNKT cells or they may comprise engineered iNKT cells.

Engineered iNKT cells may be used to treat a patient. In some embodiments, methods include introducing one or more additional nucleic acids into the cell population, which may or may not have been previously frozen and thawed. This use provides one of the advantages of creating an off-the-shelf iNKT cell. In particular embodiments, the one or more additional nucleic acids encode one or more therapeutic gene products. Examples of therapeutic gene products include at least the following: 1. Antigen recognition molecules, e.g. CAR (chimeric antigen receptor) and/or TCR (T cell receptor); 2. Co-stimulatory molecules, e.g. CD28, 4-1BB, 4-1BBL, CD40, CD40L, ICOS; and/or 3. Cytokines, e.g. IL-1α, IL-1β, IL-2, IL-4, IL-6, IL-7, IL-9, IL-15, IL-12, IL-17, IL-21, IL-23, IFN-γ, TNF-α, TGF-β, G-CSF, GM-CSF; 4. Transcription factors, e.g. T-bet, GATA-3, RORγt, FOXP3, and Bcl-6. Therapeutic antibodies are included, as are chimeric antigen receptors, single chain antibodies, monobodies, humanized, antibodies, bi-specific antibodies, single chain FV antibodies or combinations thereof.

In some embodiments, there are methods of preparing a cell population comprising engineered invariant natural killer (iNKT) T cells comprising: a) selecting CD34+ cells from human peripheral blood cells (PBMCs); b) culturing the CD34+ cells with medium comprising growth factors that include c-kit ligand, flt-3 ligand, and human thrombopoietin (TPO); c) transducing the selected CD34+ cells with a lentiviral vector comprising a nucleic acid sequence encoding α-TCR, β-TCR, thymidine kinase, and a suicide gene such as sr39TK; d) introducing into the selected CD34+ cells Cas9 and gRNA for beta 2 microglobulin (B2M) and/or CTIIA to disrupt expression of B2M and/or CTIIA; e) culturing the transduced cells for 2-12 (such as 2-10 or 6-12) weeks with an irradiated stromal cell line expressing an exogenous Notch ligand to expand iNKT cells in a 3D aggregate cell culture; f) selecting iNKT cells lacking surface expression of HLA-I and/or HLA-II molecules; and, g) culturing the selected iNKT cells with irradiated feeder cells loaded with α-GC.

In some embodiments, there are engineered iNKT cells produced by a method comprising: a) selecting CD34+ cells from human peripheral blood cells (PBMCs); b) culturing the CD34+ cells with medium comprising growth factors that include c-kit ligand, flt-3 ligand, and human thrombopoietin (TPO); c) transducing the selected CD34+ cells with a lentiviral vector comprising a nucleic acid sequence encoding α-TCR, β-TCR, thymidine kinase, and a reporter gene product; d) introducing into the selected CD34+ cells Cas9 and gRNA for beta 2 microglobulin (B2M) and/or CTIIA to eliminate expression of B2M or CTIIA; e) culturing the transduced cells for 2-10 weeks with an irradiated stromal cell line expressing an exogenous Notch ligand to expand iNKT cells in a 3D aggregate cell culture; f) selecting iNKT cells lacking expression of B2M and/or CTIIA; and, g) culturing the selected iNKT cells with irradiated feeder cells.

Methods of treating patients with an iNKT cell or cell population are also provided. In certain embodiments, the patient has cancer. In some embodiments, the patient has a disease or condition involving inflammation, which, in some embodiments, excludes cancer. In specific embodiments, the patient has an autoimmune disease or condition. In particular aspects, the cells or cell population is allogeneic with respect to the patient. In additional embodiments, the patient does not exhibit signs of rejection or depletion of the cells or cell population. Some therapeutic methods further include administering to the patient a stimulatory molecule (e.g. α-GC, alone or loaded onto APCs) that activates iNKT cells, or a compound that initiates the suicide gene product.

In some embodiments, the cancer being treated with the engineered iNKT cells comprises leukemia. In some embodiments, the cancer being treated with the engineered iNKT cells comprises chronic myelogenous leukemia cells. In some embodiments, the cancer being treated with the engineered iNKT cells comprises a blood cancer. In some embodiments, the cancer being treated with the engineered iNKT cells comprises multiple myeloma. In some embodiments, the cancer being treated with the engineered iNKT cells comprises prostate cancer. In some embodiments, the cancer being treated with the engineered iNKT cells comprises lung cancer.

Treatment of a cancer patient with the iNKT cells may result in tumor cells of the cancer patient being killed after administering the cells or cell population to the patient. Treatment of an inflammatory disease or condition may result in reducing inflammation. In other embodiments, a patient with an autoimmune disease or condition may experience an improvement in symptoms of the disease or condition or may experience other therapeutic benefits from the iNKT cells. Combination treatments with iNKT cells and standard therapeutic regimens or other immunotherapy regimen(s) may be employed.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawing, in which:

FIG. 1 illustrates a schematic of an example of production and use of an off-the-shelf universal hematopoietic stem cell (HSC)-engineered iNKT (^{U}HSC-iNKT) cell adoptive therapy;

FIGS. 2A-2D concern generation of human HSC-engineered iNKT cells in a BLT (human bone marrow-liver-thymus engrafted NOD/SCID/γc^{-/-} mice) humanized mouse model. (2A) Example of an experimental design. (2B) FACS plots of spleen cells. HSC-iNKT^{BLT}: human HSC-engineered iNKT cells generated in BLT mice. hTc: human conventional T cells. FIGS. 2C-2D show generation of human HSC-engineered NY-ESO-1 specific conventional T cells in an Artificial Thymic Organoid (ATO) in vitro culture system. (2C) Example of an experimental design. (2D) Cell yield (n=3-6). **P<0.01, by Student's t test;

FIGS. 3A-3D demonstrate an initial CMC study in which there is generation of human HSC-engineered iNKT cells in a robust and high-yield two-stage ATO-αGC in vitro culture system. (HSC-iNKT^{ATO} cells were studied as a therapeutic surrogate.) HSC-iNKT^{ATO}: human HSC-engineered iNKT cells generated in ATO culture.) (3A) A 2-stage ATO-αGC in vitro culture system. ATO: Artificial Thymic Organoid; αGC: alpha-Galactosylceramide, a potent agonist ligand that specifically stimulates iNKT cells. (3B) Generation of HSC-iNKT^{ATO} cells at the ATO culture stage. 6B11 is a monoclonal antibody that specifically binds to iNKT TCR. (3C) Expansion of HSC-iNKT^{ATO} cells at the PBMC/αGC culture stage. (3D) HSC-iNKT^{ATO} cell outputs;

FIGS. 4A-4B provide an initial pharmacology study of the phenotype and functionality of human HSC-engineered iNKT cells. (HSC-iNKT^{ATO} and HSC-iNKT^{BLT} cells were studied as therapeutic surrogates.) (4A) Surface FACS staining. (4B) Intracellular FACS staining. PBMC-iNKT: endogenous iNKT cells expanded in vitro from healthy donor PBMCs; PBMC-Tc: endogenous conventional T cells from healthy donor PBMCs;

FIGS. 5A-5K provide an initial efficacy study of Tumor Killing Efficacy of Human HSC-Engineered iNKT cells. (HSC-iNKT^{ATO} and HSC-iNKT^{BLT} cells were studied as therapeutic surrogates.) (5A-5F) Blood cancer model. (5A) MM.1S-hCD1d-FG human multiple myeloma (MM) cell line. (5B) In vitro tumor killing assay. (5C) Luciferase activity analysis of the in vitro tumor killing (n=3). (5D) In vivo tumor killing assay using an NSG mouse human MM metastasis model. (E-F) Live animal bioluminescence imaging (BLI) analysis of the in vivo tumor killing. Representative BLI images of day 14 (5E) and the time course measurement of total body luminescence (TBL; 5F) are shown (n=3-4). (5G-5K) Solid tumor model. (5G) A375-hCD1d-FG human melanoma cell line. (5H) In vivo tumor killing assay using an NSG mouse human melamona solid tumor model. (5I) Tumor weight (day 25). (5J) FACS plots showing the HSC-iNKT^{BLT} cell infiltration into the tumor site (day 25). (5K) Quantification of J (n=4). **P<0.01, ***P<0.001, by Student's t test.

FIGS. 6A-6C show an initial safety study of Toxicology/Tumorigenicity. (HSC-iNKT^{BLT} cells were studied as a therapeutic surrogate.) (6A) Mouse body weight (n=9-10). ns, not significant, by Student's t test. (6B) Mouse survival rate (n=9-10). (6C) Mouse pathology. Various tissues were collected and analyzed by the UCLA Pathology Core (n=9-10).

FIGS. 7A-7D provide an initial safety study of sr39TK gene for PET imaging and safety control. (HSC-iNKT^{BLT} cells were studied as a therapeutic surrogate.) (7A) Experimental design. (7B) PET/CT images of the BLT-iNKT^{TK} mice prior to and post GCV treatment (n=4-5). (7C) FACS plots showing the effective and specific depletion of HSC-iNKT^{BLT} cells post GCV treatment (n=4-5). (7D) Quantification of the FACS plots in 7C (n=4-5). ns, not significant; **P<0.01; by Student's t test.

FIGS. 8A-8F illustrate an example of a manufacturing process to produce the ^{U}HSC-iNKT cells. (8A) Experimental design. (8B) Lenti/iNKT-sr39TK vector-mediated iNKT TCR expression in HSCs. (8C) CRISPR-Cas9/B2M-CIITA-gRNAs complex-mediated knockout of the HLA-I/II expression in HSCs. (8D) Diagram showing the purification step between the Stage 1 culture and Stage 2 culture. (8E) 2M2/Tü39 mAb-mediated MACS negative-selection of HLA-I/II^{neg} cells. (8F) 6B11 mAb-mediated MACS positive-selection of HSC-iNKT^{ATO} cells;

FIGS. 9A-9E provide an example of a mechanism of action (MOA) Study. (9A) Possible mechanisms used by iNKT cells to target tumor. (9B-9C) Study of CD1d/TCR-mediated direct killing of tumor cells. (9B) Experimental design; (9C) Killing of MM.1S-hCD1d-FG human multiple myeloma cells (n=3). (9D-9E) Study of CD1d-independant targeting of tumor cells through activating NK cells. (9D) Experimental design; (9E) Killing of K562 tumor cells (n=2). Irradiated PBMCs loaded with aGC were used as antigen-presenting cells (APCs) ns, not significant, *P<0.05, **P<0.01, ****P<0.0001, by one-way ANAVO.;

FIGS. 10A-10G demonstrate safety considerations. (10A) Possible GvHD and HvG responses and the engineered safety control strategies. (10B) An in vitro mixed lymphocyte culture (MLC) assay for the study of GvHD responses. (10C) IFN-γ production in MLC assay showing no GvHD response induced by HSC-iNKT^{ATO} cells (n=3). PBMCs from 3 different healthy donors were included as responders. (10D) An in vitro mixed lymphocyte culture (MLC) assay for the study of HvG response. (10E) IFN-γ production in MLC assay showing minor HvG responses against HSC-iNKT^{ATO} cells (n=3). PBMCs from 2 different healthy donors were used in the experiment. (10F) HSC-iNKT^{BLT} cells were resistant to killing by mismatched-donor NK cells in an in vitro mixed NK/iNKT culture. (10G) An in vivo mixed lymphocyte adoptive transfer (MLT) assay to study the GvHD and HvD responses. ns, not significant, **P<0.01, ***P<0.001, ****P<0.0001, by one-way ANAVO.

FIGS. 11A-11G demonstrate examples of Combination therapy. (11A) Experimental design to study the ^{U}HSC-iNKT cell therapy in combination with the checkpoint blockade therapy. (11B)^{UHSC}CAR-iNKT cell. (11C) A375-hCD1d-hCD19-FG human melanoma cell line. (11D) Experimental design to study the anti-tumor efficacy of the ^{UHSC}CAR-iNKT cells. (11E)^{UHSC}TCR-iNKT cells. (11F) A375-hCD1d-A2/ESO-FG human melanoma cell line. (11G) Experimental design to study the anti-tumor efficacy of the ^{UHSC}TCR-iNKT cells.

FIG. 12 illustrates an example of a Pharmacokinetics/Pharmacodynamics (PK/PD) study.

FIG. 13 shows one example of an iNKT-sr39TK Lentiviral vector.

FIG. 14 illustrates one example of a cell manufacturing process for production of ^{U}HSC-iNKT cells.

FIG. 15. Phenotype and functionality of HSC-iNKT cells. Representative FACS plots are presented, showing the surface staining of NK activation receptors (NKG2D and DNAM-1) and inhibitory receptors (KIR), and intracellular staining of cytotoxic molecules (Perforin and Granzyme B). Native NK cells isolated from the peripheral blood of healthy human donors (PBMC-NK cells) were included as a control.

FIG. 16A-G. In vitro efficacy and MOA study. (A) Experimental design to study NK cell-like tumor cell killing by HSC-iNKT cells. Multiple human tumor cell lines were used in this study, and were engineered to overexpress firefly luciferase (Flue) and enhanced green fluorescent protein (EGFP) dual reporters to enable sensitive measurement of tumor killing using luciferase activity assay. A375-FG, engineered human melanoma tumor cell line; K562-FG, engineered human chronic myelogenous leukemia cell line; MM.1S-FG, engineered human multiple myeloma cell line; H292-FG, engineered human lung cancer cell line; PC3-FG, engineered human prostate cancer cell line. (B-F) Luciferase activity analysis of the in vitro killing of various human tumor cells by fresh or frozen/thawed HSC-iNKT cells. PBMC-NK cells, fresh or frozen/thawed, were included as controls. (G) Luciferase activity analysis of tumor cell killing efficacy by HSC-iNKT cells in the presence of NKG2D or/and DNAM-1 blocking antibodies. Representative of 2 experiments. Data are presented as the mean±SEM. ns, not significant, *P<0.05, **P<0.01, ***P<0.001, ****P<0.0001, by 1-way ANOVA.

FIG. 17A-D. In vivo efficacy study. (A) Experimental design. (B) Quantification of total body luminescence (TBL) over time (n=5). (C) Measurement of tumor size over time (n=5). (D) Measurement of tumor weight at day 26 (n=5). Representative of 2 experiments. Data are presented as the mean±SEM. ns, not significant, *P<0.05, **P<0.01, ***P<0.001, ****P<0.0001, by Student's t test.

### DETAILED DESCRIPTION

### I. Examples of Definitions

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the invention may “consist essentially of” or “consist of” one or more sequences of the invention, for example. Some embodiments of the invention may consist of or consist essentially of one or more elements, method steps, and/or methods of the invention. It is contemplated that any method or composition described herein can be implemented with respect to any other method or composition described herein.

The present disclosure encompasses “HSC-iNKT cells”, invariant natural killer T (iNKT) cells engineered from hematopoietic stem cells (HSCs) and/or hematopoietic progenitor cells (HPCs), and methods of making and using thereof. As used herein, “HSCs” is used to refer to HSCs, HPCs, or both HSCs and HPCs.

The term “therapeutically effective amount” as used herein refers to an amount that is effective to alleviate, ameliorate, or prevent at least one symptom or sign of a disease or condition to be treated.

The term “exogenous TCR” refers to a TCR gene or TCR gene derivative that is transferred (i.e. by way of gene transfer/transduction/transfection techniques) into the cell or is the progeny of a cell that has received a transfer of a TCR gene or gene derivative. The exogenous TCR genes are inserted into the genome of the recipient cell. In some embodiments, the insertion is random insertion. Random insertion of the TCR gene is readily achieved by methods known in the art. In some embodiments, the TCR genes are inserted into an endogenous loci (such as an endogenous TCR gene loci). In some embodiments, the cells comprise one or more TCR genes that are inserted at a loci that is not the endogenous loci. In some embodiments, the cells further comprise heterologous sequences such as a marker or resistance gene.

The term “chimeric antigen receptor” or “CAR” refers to engineered receptors, which graft an arbitrary specificity onto an immune effector cell. These receptors are used to graft the specificity of a monoclonal antibody onto a T cell; with transfer of their coding sequence facilitated by retroviral or lentiviral vectors. The receptors are called chimeric because they are composed of parts from different sources. The most common form of these molecules are fusions of single-chain variable fragments (scFv) derived from monoclonal antibodies, fused to CD3-zeta transmembrane and endodomain; CD28 or 41BB intracellular domains, or combinations thereof. Such molecules result in the transmission of a signal in response to recognition by the scFv of its target. An example of such a construct is 14g2a-Zeta, which is a fusion of a scFv derived from hybridoma 14g2a (which recognizes disialoganglioside GD2). When T cells express this molecule (as an example achieved by oncoretroviral vector transduction), they recognize and kill target cells that express GD2 (e.g. neuroblastoma cells). To target malignant B cells, investigators have redirected the specificity of T cells using a chimeric immunoreceptor specific for the B-lineage molecule, CD19. The variable portions of an immunoglobulin heavy and light chain are fused by a flexible linker to form a scFv. This scFv is preceded by a signal peptide to direct the nascent protein to the endoplasmic reticulum and subsequent surface expression (this is cleaved). A flexible spacer allows the scFv to orient in different directions to enable antigen binding. The transmembrane domain is a typical hydrophobic alpha helix usually derived from the original molecule of the signalling endodomain which protrudes into the cell and transmits the desired signal.

The term “antigen” refers to any substance that causes an immune system to produce antibodies against it, or to which a T cell responds. In some embodiments, an antigen is a peptide that is 5-50 amino acids in length or is at least, at most, or exactly 5, 10, 15, 20, 25, 30, 35, 40, 45, 50, 55, 60, 65, 70, 75, 80, 85, 90, 95, 100, 125, 150, 175, 200, 250, or 300 amino acids, or any derivable range therein.

The term “allogeneic to the recipient” is intended to refer to cells that are not isolated from the recipient. In some embodiments, the cells are not isolated from the patient. In some embodiments, the cells are not isolated from a genetically matched individual (such as a relative with compatible genotypes).

The term “inert” refers to one that does not result in unwanted clinical toxicity. This could be either on-target or off-target toxicity. “Inertness” can be based on known or predicted clinical safety data.

The term “xeno-free (XF)” or “animal component-free (ACF)” or “animal free,” when used in relation to a medium, an extracellular matrix, or a culture condition, refers to a medium, an extracellular matrix, or a culture condition which is essentially free from heterogeneous animal-derived components. For culturing human cells, any proteins of a non-human animal, such as mouse, would be xeno components. In certain aspects, the xeno-free matrix may be essentially free of any non-human animal-derived components, therefore excluding mouse feeder cells or Matrigel™. Matrigel™ is a solubilized basement membrane preparation extracted from the Engelbreth-Holm-Swarm (EHS) mouse sarcoma, a tumor rich in extracellular matrix proteins to include laminin (a major component), collagen IV, heparin sulfate proteoglycans, and entactin/nidogen.

The term “defined,” when used in relation to a medium, an extracellular matrix, or a culture condition, refers to a medium, an extracellular matrix, or a culture condition in which the nature and amounts of approximately all the components are known.

A “chemically defined medium” refers to a medium in which the chemical nature of approximately all the ingredients and their amounts are known. These mediva are also called synthetic media. Examples of chemically defined media include TeSR™.

Cells are “substantially free” of certain reagents or elements, such as serum, signaling inhibitors, animal components or feeder cells, exogenous genetic elements or vector elements, as used herein, when they have less than 10% of the element(s), and are “essentially free” of certain reagents or elements when they have less than 1% of the element(s). However, even more desirable are cell populations wherein less than 0.5% or less than 0.1% of the total cell population comprise exogenous genetic elements or vector elements.

A culture, matrix or medium are “essentially free” of certain reagents or elements, such as serum, signaling inhibitors, animal components or feeder cells, when the culture, matrix or medium respectively have a level of these reagents lower than a detectable level using conventional detection methods known to a person of ordinary skill in the art or these agents have not been extrinsically added to the culture, matrix or medium. The serum-free medium may be essentially free of serum.

“Peripheral blood cells” refer to the cellular components of blood, including red blood cells, white blood cells, and platelets, which are found within the circulating pool of blood.

“Hematopoietic stem and progenitor cells” or “hematopoietic precursor cells” refers to cells that are committed to a hematopoietic lineage but are capable of further hematopoietic differentiation and include hematopoietic stem cells, multipotential hematopoietic stem cells (hematoblasts), myeloid progenitors, megakaryocyte progenitors, erythrocyte progenitors, and lymphoid progenitors. “Hematopoietic stem cells (HSCs)” are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T-cells, B-cells, NK-cells). In this disclosure, HSCs refer to both “hematopoietic stem and progenitor cells” and “hematopoietic precursor cells”.

The hematopoietic stem and progenitor cells may or may not express CD34. The hematopoietic stem cells may co-express CD133 and be negative for CD38 expression, positive for CD90, negative for CD45RA, negative for lineage markers, or combinations thereof. Hematopoietic progenitor/precursor cells include CD34(+)/CD38(+) cells and CD34(+)/CD45RA(+)/lin(−)CD10+(common lymphoid progenitor cells), CD34(+)CD45RA(+)lin(−) CD10(−)CD62L(hi) (lymphoid primed multipotent progenitor cells), CD34(+)CD45RA(+)lin(−)CD10(−)CD123+ (granulocyte-monocyte progenitor cells), CD34(+)CD45RA(−)lin(−)CD10(−)CD123+ (common myeloid progenitor cells), or CD34(+)CD45RA(−)lin(−)CD10(−)CD123− (megakaryocyte-erythrocyte progenitor cells).

A “vector” or “construct” (sometimes referred to as gene delivery or gene transfer “vehicle”) refers to a macromolecule, complex of molecules, or viral particle, comprising a polynucleotide to be delivered to a host cell, either in vitro or in vivo. The polynucleotide can be a linear or a circular molecule.

A “plasmid”, a common type of a vector, is an extra-chromosomal DNA molecule separate from the chromosomal DNA which is capable of replicating independently of the chromosomal DNA. In certain cases, it is circular and double-stranded.

By “expression construct” or “expression cassette” is meant a nucleic acid molecule that is capable of directing transcription. An expression construct includes, at the least, a promoter or a structure functionally equivalent to a promoter. Additional elements, such as an enhancer, and/or a transcription termination signal, may also be included.

The term “exogenous,” when used in relation to a protein, gene, nucleic acid, or polynucleotide in a cell or organism refers to a protein, gene, nucleic acid, or polynucleotide which has been introduced into the cell or organism by artificial means, or in relation a cell refers to a cell which was isolated and subsequently introduced to other cells or to an organism by artificial means. An exogenous nucleic acid may be from a different organism or cell, or it may be one or more additional copies of a nucleic acid which occurs naturally within the organism or cell. An exogenous cell may be from a different organism, or it may be from the same organism. By way of a non-limiting example, an exogenous nucleic acid is in a chromosomal location different from that of natural cells, or is otherwise flanked by a different nucleic acid sequence than that found in nature.

The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

A “gene,” “polynucleotide,” “coding region,” “sequence,” “segment,” “fragment,” or “transgene” which “encodes” a particular protein, is a nucleic acid molecule which is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “cell” is herein used in its broadest sense in the art and refers to a living body which is a structural unit of tissue of a multicellular organism, is surrounded by a membrane structure which isolates it from the outside, has the capability of self-replicating, and has genetic information and a mechanism for expressing it. Cells used herein may be naturally-occurring cells or artificially modified cells (e.g., fusion cells, genetically modified cells, etc.).

As used herein, the term “stem cell” refers to a cell capable of self-replication and pluripotency or multipotency. Typically, stem cells can regenerate an injured tissue. Stem cells herein may be, but are not limited to, embryonic stem (ES) cells, induced pluripotent stem cells or tissue stem cells (also called tissue-specific stem cell, or somatic stem cell).

“Embryonic stem (ES) cells” are pluripotent stem cells derived from early embryos. An ES cell was first established in 1981, which has also been applied to production of knockout mice since 1989. In 1998, a human ES cell was established, which is currently becoming available for regenerative medicine.

Unlike ES cells, tissue stem cells have a limited differentiation potential. Tissue stem cells are present at particular locations in tissues and have an undifferentiated intracellular structure. Therefore, the pluripotency of tissue stem cells is typically low. Tissue stem cells have a higher nucleus/cytoplasm ratio and have few intracellular organelles. Most tissue stem cells have low pluripotency, a long cell cycle, and proliferative ability beyond the life of the individual. Tissue stem cells are separated into categories, based on the sites from which the cells are derived, such as the dermal system, the digestive system, the bone marrow system, the nervous system, and the like. Tissue stem cells in the dermal system include epidermal stem cells, hair follicle stem cells, and the like. Tissue stem cells in the digestive system include pancreatic (common) stem cells, liver stem cells, and the like. Tissue stem cells in the bone marrow system include hematopoietic stem cells, mesenchymal stem cells, and the like. Tissue stem cells in the nervous system include neural stem cells, retinal stem cells, and the like.

“Induced pluripotent stem cells,” commonly abbreviated as iPS cells or iPSCs, refer to a type of pluripotent stem cell artificially prepared from a non-pluripotent cell, typically an adult somatic cell, or terminally differentiated cell, such as fibroblast, a hematopoietic cell, a myocyte, a neuron, an epidermal cell, or the like, by introducing certain factors, referred to as reprogramming factors.

As used herein, “isolated” for example, with respect to cells and/or nucleic acids means altered or removed from the natural state through human intervention.

“Pluripotency” refers to a stem cell that has the potential to differentiate into all cells constituting one or more tissues or organs, or particularly, any of the three germ layers: endoderm (interior stomach lining, gastrointestinal tract, the lungs), mesoderm (muscle, bone, blood, urogenital), or ectoderm (epidermal tissues and nervous system). “Pluripotent stem cells” used herein refer to cells that can differentiate into cells derived from any of the three germ layers, for example, direct descendants of totipotent cells or induced pluripotent cells.

By “operably linked” with reference to nucleic acid molecules is meant that two or more nucleic acid molecules (e.g., a nucleic acid molecule to be transcribed, a promoter, and an enhancer element) are connected in such a way as to permit transcription of the nucleic acid molecule. “Operably linked” with reference to peptide and/or polypeptide molecules is meant that two or more peptide and/or polypeptide molecules are connected in such a way as to yield a single polypeptide chain, i.e., a fusion polypeptide, having at least one property of each peptide and/or polypeptide component of the fusion. The fusion polypeptide is particularly chimeric, i.e., composed of heterologous molecules.

Embodiments of the disclosure concern HSC cells engineered to function as iNKT cells with an NKT cell T cell receptor (TCR) and that also have imaging and suicide targeting capabilities and are resistant to host immune cell-targeted depletion. Such cells are generated in an Artificial Thymic Organoid (ATO) in vitro culture system that supports the differentiation of the TCR-engineered HSCs into clonal T cells at high-efficiency and high yield.

### II. Universal Hematopoietic Stem Cell (HSC) Engineered Invariant NKT Cells (^{U}HSC-iNKT Cells)

Embodiments of the disclosure utilize cells (such as HSCs) that are modified to function as invariant NKT cells and that are engineered to have one or more characteristics that render the cells suitable for universal use (use for individuals other than the individual from which the original cells were obtained) without deleterious immune reaction in a recipient of the cells. The present disclosure encompasses engineered invariant natural killer T (iNKT) cells comprising a nucleic acid comprising i) all or part of an iNKT alpha T-cell receptor gene; ii) all or part of an iNKT beta T-cell receptor gene, and iii) a suicide gene, wherein the genome of the cell has been altered to eliminate surface expression of at least one HLA-I or HLA-II molecule.

### A. iNKT Cells

In particular embodiments, the engineered iNKT cells of the disclosure are produced from other types of cells to facilitate their activity as iNKT cells. iNKT cells are a small subpopulation of αβT lymphocytes that have several unique features that make them useful for off-the-shelf cellular therapy, including at least for cancer therapy. Non-iNKT cells are engineered to function as iNKT cells because of the following advantages of iNKT cells:

1) iNKT cells have the remarkable capacity to target multiple types of cancer independent of tumor antigen- and MHC-restrictions (Fujii et al., 2013). iNKT cells recognize glycolipid antigens presented by non-polymorphic CD1d, which frees them from MHC-restriction. Although the natural ligands of iNKT cells remain to be identified, it is suggested that iNKT cells can recognize certain conserved glycolipid antigens derived from many tumor tissues. iNKT cells can be stimulated through recognizing these glycolipid antigens that are either directly presented by CD1d⁺ tumor cells, or indirectly cross-presented by tumor infiltrating antigen-presenting cells (APCs) like macrophages or dendritic cells (DCs) in case of CD1d tumors. Thus, iNKT cells can respond to both CD1d⁺ and CD1d⁻ tumors.

2) iNKT cells can employ multiple mechanisms to attack tumor cells (Vivier et al., 2012; Fujii et al., 2013). iNKT cells can directly kill CD1d⁺ tumor cells through cytotoxicity, but their most potent anti-tumor activities come from their immune adjuvant effects. iNKT cells remain quiescent prior to stimulation, but after stimulation, they immediately produce large amounts of cytokines, mainly IFN-γ. IFN-γ activates NK cells to kill MHC-negative tumor target cells. Meanwhile, iNKT cells also activate DCs that then stimulate CTLs to kill MHC-positive tumor target cells. Therefore, iNKT cell-induced anti-tumor immunity can effectively target multiple types of cancer independent of tumor antigen- and MHC-restrictions, thereby effectively blocking tumor immune escape and minimizing the chance of tumor recurrence.

3) iNKT cells do not cause graft-versus-host disease (GvHD). Because iNKT cells do not recognize mismatched MHC molecules and protein autoantigens, these cells are not expected to cause GvHD. This notion is strongly supported by clinical data analyzing donor-derived iNKT cells in blood cancer patients receiving allogeneic bone marrow or peripheral blood stem cell transplantation. These clinical data showed that the levels of engrafted allogenic iNKT cells in patients correlated positively with graft-versus-leukemia effects and negatively with GvHD (Haraguchi et al., 2004; de Lalla et al., 2011).

4) iNKT cells can be engineered to avoid host-versus-graft (HvG) depletion. The availability of powerful gene-editing tools like the CRISPR-Cas9 system make it possible to genetically modify iNKT cells to make them resistant to host immune cell-targeted depletion: knockout of beta 2-microglobulin (B2M) gene will ablate HLA-I molecule expression on iNKT cells to avoid host CD8⁺ T cell-mediated killing; knockout of CIITA gene will ablate HLA-II molecule expression on iNKT cells to avoid CD4⁺ T cell-mediated killing. Both B2M and CIITA genes are approved good targets for the CRISPR-Cas9 system in human primary cells (Ren et al., 2017; Abrahimi et al., 2015). Ablation of HLA-I expression on iNKT cells may make them targets of host NK cells. However, iNKT cells seem to naturally resist allogenic NK cell killing. Nonetheless, if necessary, the concern can be addressed by delivering into iNKT cells an NK-inhibitory gene like HLA-E.

5) iNKT cells have strong relevance to cancer. There is compelling evidence to suggest a significant role of iNKT cells in tumor surveillance in mice, in which iNKT cell defects predispose them to cancer and the adoptive transfer or stimulation of iNKT cells can provide protection against cancer (Vivier et al., 2012; Berzins et al., 2011). In humans, iNKT cell frequency is decreased in patients with solid tumors (including melanoma, colon, lung, breast, and head and neck cancers) and blood cancers (including leukemia, multiple myeloma, and myelodysplastic syndromes), while increased iNKT cell numbers are associated with a better prognosis (Berzins et al., 2011). There are also instances wherein the administration of α-GalCer-loaded DCs and ex vivo expanded autologous iNKT cells has led to promising clinical benefits in patients with lung cancer and head and neck cancer, although the increases of iNKT cells have been transient and the clinical benefits have been short-term, likely due to the limited number of iNKT cells used for transfer and the depletion of these cells thereafter (Fujii et al., 2012; Yamasaki et al., 2011). Therefore, it is plausible to propose that an “off-the-shelf” iNKT cellular product enabling the transfer into patients sufficient iNKT cells at multiple doses may provide patients with the best chance to exploit the full potential of iNKT cells to battle their diseases.

However, the development of an allogenic off-the-shelf iNKT cellular product is greatly hindered by their availability—these cells are of extremely low number and high variability in humans (˜0.001-1% in human blood), making it very difficult to grow therapeutic numbers of iNKT cells from blood cells of allogenic human donors. A novel method that can reliably generate homogenous population of iNKT cells at large quantity is thus key to developing an off-the-shelf iNKT cell therapy.

Given this lack of sufficient amounts of iNKT cells for clinical applications, embodiments of the disclosure encompass the engineering of non-iNKT cells such that the resultant engineered cell functions as an iNKT cell. In specific embodiments, the cells that function as iNKT cells are further modified to have one or more desired characteristics. In specific embodiments, non-iNKT cells are modified genetically through transduction of the non-iNKT cell to express an iNKT T cell receptor (TCR).

### B. iNKT Cells Produced from HSCs Cells

In embodiments of the disclosure, iNKT cells produced from other types of cells are engineered to have one or more characteristics to render them suitable for universal use. In specific embodiments, a cell is genetically modified to contain at least one exogenous invariant natural killer T cell receptor (iNKT TCR) nucleic acid molecule. In some embodiments, the cell is a hematopoietic stem cell. In some embodiments, the cell is a hematopoietic progenitor cell. In some embodiments, the cell is a human cell. In some embodiments, the cell is a CD34⁺ cell. In some embodiments, the cell is a human CD34⁺ cell. In some embodiments, the cell is a recombinant cell. In some embodiments, the cell is of a cultured strain.

In some embodiments, the iNKT TCR nucleic acid molecule is from a human invariant natural killer T cell. In some embodiments, the iNKT TCR nucleic acid molecule comprises one or more nucleic acid sequences obtained from a human iNKT TCR. In some embodiments, the iNKT TCR nucleic acid sequence can be obtained from any subset of iNKT cells, such as the CD4/DN/CD8 subsets or the subsets that produce Th1, Th2, or Th17 cytokines, and includes double negative iNKT cells. In some embodiments, the iNKT TCR nucleic acid sequence is obtained from an iNKT cell from a donor who had or has a cancer such as melanoma, kidney cancer, lung cancer, prostate cancer, breast cancer, lymphoma, leukemia, a hematological malignancy, and the like. In some embodiments, the iNKT TCR nucleic acid molecule has a TCR-alpha sequence from one iNKT cell and a TCR-beta sequence from a different iNKT cell. In some embodiments, the iNKT cell from which the TCR-alpha sequence is obtained and the iNKT cell from which the TCR-beta sequence is obtained are from the same donor. In some embodiments, the donor of the iNKT cell from which the TCR-alpha sequence is obtained is different from the donor of the iNKT cell from which the TCR-beta sequence is obtained. In some embodiments, the TCRalpha sequence and/or the TCR-beta sequence are codon optimized for expression. In some embodiments, the TCR-alpha sequence and/or the TCR-beta sequence are modified to encode a polypeptide having one or more amino acid substitutions, deletions, and/or truncations compared to the polypeptide encoded by the unmodified sequence. In some embodiments, the iNKT TCR nucleic acid molecule encodes a T cell receptor that recognizes alpha-galactosylceramide (alpha-GalCer) presented on CD1d. In some embodiments, the iNKT TCR nucleic acid molecule comprises one or more sequences selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 18, SEQ ID NO: 19, SEQ ID NO: 21, SEQ ID NO: 22, SEQ ID NO: 24, SEQ ID NO: 25, SEQ ID NO: 27, SEQ ID NO: 28, SEQ ID NO: 30, SEQ ID NO: 31, SEQ ID NO: 33, SEQ ID NO: 34, SEQ ID NO: 36, SEQ ID NO: 37, SEQ ID NO: 39, SEQ ID NO: 40, SEQ ID NO: 42, SEQ ID NO: 43, SEQ ID NO: 45, SEQ ID NO: 46, SEQ ID NO: 48, SEQ ID NO: 49, SEQ ID NO: 51, SEQ ID NO: 52, SEQ ID NO: 54, SEQ ID NO: 55, SEQ ID NO: 57, SEQ ID NO: 58, SEQ ID NO: 60, SEQ ID NO: 61, SEQ ID NO:63, and SEQ ID NO:64. In some embodiments, the iNKT TCR nucleic acid molecule encodes a polypeptide comprising an amino acid sequence selected from the group consisting of: SEQ ID NO: 20, SEQ ID NO: 23, SEQ ID NO: 26, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50, SEQ ID NO: 53, SEQ ID NO: 56, SEQ ID NO: 59, SEQ ID NO: 62, and SEQ ID NO:65. In some embodiments, the engineered cell lacks exogenous oncogenes, such as Oct4, Sox2, Klf, c-Myc, and the like.

In some embodiments, the engineered cell is a functional iNKT cell. In some embodiments, the engineered cell is capable of producing one or more cytokines and/or chemokines such as IFN-gamma, TNF-alpha, TGF-beta, GM-CSF, IL-2, IL-4, IL-5, IL-6, IL-10, IL-13, IL-17, IL-21, RANTES, Eotaxin, MIP-1-alpha, MIP-1-beta, and the like.

Donor HSPCs can be obtained from the bone marrow, peripheral blood, amniotic fluid, or umbilical cord blood of a donor. The donor can be an autologous donor, i.e., the subject to be treated with the HSPC-iNKT cells, or an allogenic donor, i.e., a donor who is different from the subject to be treated with the HSPC-iNKT cells. In embodiments where the donor is an allogenic donor, the tissue (HLA) type of the allogenic donor preferably matches that of the subject being treated with the HSPC-iNKT cells derived from the donor HSPCs.

According to the present disclosure, an HSPC is transduced with one or more exogenous iNKT TCR nucleic acid molecules. As used herein, an “iNKT TCR nucleic acid molecule” is a nucleic acid molecule that encodes an alpha chain of an iNKT T cell receptor (TCR-alpha-), a beta chain of an iNKT T cell receptor (TCR-beta), or both. As used herein, an “iNKT T cell receptor” is one that is expressed in an iNKT cell and recognizes alpha-GalCer presented on CD1d. The TCR-alpha and TCR-beta sequences of iNKT TCRs can be cloned and/or recombinantly engineered using methods in the art. For example, an iNKT cell can be obtained from a donor and the TCR.alpha. and .beta. genes of the iNKT cell can be cloned as described herein. The iNKT TCR to be cloned can be obtained from any mammalian including humans, non-human primates such monkeys, mice, rats, hamsters, guinea pigs, and other rodents, rabbits, cats, dogs, horses, bovines, sheep, goat, pigs, and the like. In some embodiments, the iNKT TCR to be cloned is a human iNKT TCR. In some embodiments, the iNKT TCR clone comprises human iNKT TCR sequences and non-human iNKT TCR sequences.

In some embodiments, the cloned TCR can have a TCR-alpha chain from one iNKT cell and a TCR-beta chain from a different iNKT cell. In some embodiments, the iNKT cell from which the TCR-alpha chain is obtained and the iNKT cell from which the TCR-beta chain is obtained are from the same donor. In some embodiments, the donor of the iNKT cell from which the TCR-alpha chain is obtained is different from the donor of the iNKT cell from which the TCR-beta chain is obtained. In some embodiments, the sequence encoding the TCR-alpha chain and/or the sequence encoding the TCR-beta chain of a TCR clone is modified. In some embodiments, the modified sequence may encode the same polypeptide sequence as the unmodified TCR clone, e.g., the sequence is codon optimized for expression. In some embodiments, the modified sequence may encode a polypeptide that has a sequence that is different from the unmodified TCR clone, e.g., the modified sequence encodes a polypeptide sequence having one or more amino acid substitutions, deletions, and/or truncations.

### C. HLA-Negative HSC-iNKT Cells with Imaging and Depletion Characteristics

In particular embodiments, iNKT cells produced from HSPCs cells are further modified to have one or more characteristics, including to render the cells suitable for allogeneic use or more suitable for allogeneic use than if the cells were not further modified to have one or more characteristics. The present disclosure encompasses ^{U}HSC-iNKT cells that are suitable for allogeneic use, if desired. In some embodiments, the HSC-iNKT cells are non-alloreactive and express an exogenous iNTK TCR. These cells are useful for “off the shelf” cell therapies and do not require the use of the patient's own iNKT or other cells. Therefore, the current methods provide for a more cost-effective, less labor-intensive cell immunotherapy.

In specific embodiments, HSC-iNKT cells are engineered to be HLA-negative to achieve safe and successful allogeneic engraftment without causing graft-versus-host disease (GvHD) and being rejected by host immune cells (HvG rejection). In specific embodiments, allogeneic HSC-iNKT cells do not express endogenous TCRs and do not cause GvHD, because the expression of the transgenic iNKT TCR gene blocks the recombination of endogenous TCRs through allelic exclusion. In particular embodiments, allogeneic ^{U}HSC-iNKT cells do not express HLA-I and/or HLA-II molecules on cell surface and resist host CD8⁺ and CD4⁺ T cell-mediated allograft depletion and sr39TK immunogen-targeting depletion.

Thus, in certain embodiments the engineered iNKT cells do not express surface HLA-I or -II molecules, achieved through disruption of genes encoding proteins relevant to HLA-I/II expression, including but not limited to beta-2-microglobulin (B2M), major histocompatibility complex II transactivator (CIITA), or HLA-I/II molecules. In some cases, the HLA-I or HLA-II are not expressed on the surface of iNKT cells because the cells were manipulated by gene editing, which may or may not involve CRISPR-Cas9.

In cases wherein the iNKT cells have been modified to exhibit one or more characteristics of any kind, the iNKT cells may comprise nucleic acid sequences from a recombinant vector that was introduced into the cells. The vector may be a non-viral vector, such as a plasmid, or a viral vector, such as a lentivirus, a retrovirus, an adeno-associated virus (AAV), a herpesvirus, or adenovirus.

The iNKT cells of the disclosure may or may not have been exposed to one or more certain conditions before, during, or after their production. In specific cases, the cells are not or were not exposed to media that comprises animal serum. The cells may be frozen. The cells may be present in a solution comprising dextrose, one or more electrolytes, albumin, dextran, and/or DMSO. Any solution in which the cells are present may be a solution that is sterile, nonpyogenic, and isotonic. The cells may have been activated and expanded by any suitable manner, such as activated with alpha-galactosylceramide (α-GC), for example.

Aspects of the disclosure relate to a human cell comprising: i) an exogenous expression or activity inhibitor of; or ii) a genomic mutation of: one or more of 132 microglobin (B2M), CIITA, TRAC, TRBC1, or TRBC2. In some embodiments, the cell comprises a genomic mutation. In some embodiments, the genomic mutation comprises a mutation of one or more endogenous genes in the cell's genome, wherein the one or more endogenous genes comprise the B2M, CIITA, TRAC, TRBC1, or TRBC2 gene. In some embodiments, the mutation comprises a loss of function mutation. In some embodiments, the inhibitor is an expression inhibitor. In some embodiments, the inhibitor comprises an inhibitory nucleic acid. In some embodiments, the inhibitory nucleic acid comprises one or more of a siRNA, shRNA, miRNA, or an antisense molecule. In some embodiments, the cells comprise an activity inhibitor. In some embodiments, following modification the cell is deficient in any detectable expression of one or more of B2M, CIITA, TRAC, TRBC1, or TRBC2 proteins. In some embodiments, the cell comprises an inhibitor or genomic mutation of B2M. In some embodiments, the cell comprises an inhibitor or genomic mutation of CIITA. In some embodiments, the cell comprises an inhibitor or genomic mutation of TRAC. In some embodiments, the cell comprises an inhibitor or genomic mutation of TRBC1. In some embodiments, the cell comprises an inhibitor or genomic mutation of TRBC2. In some embodiments, at least 90% of the genomic DNA encoding B2M, CIITA, TRAC, TRBC1, and/or TRBC2 is deleted. In some embodiments, at least or at most 5, 10, 20, 30, 40, 50, 60, 70, 80, 90, 95, 99, or 100% (or any range derivable therein) of the genomic DNA encoding B2M, CIITA, TRAC, TRBC1, and/or TRBC2 is deleted. In other embodiments, a deletion, insertion, and/or substitution is made in the genomic DNA. In some embodiments, the cell is a progeny of the human stem or progenitor cell.

The ^{U}HSC-iNKT cells that are modified to be HLA-negative may be genetically modified by any suitable manner. The genetic mutations of the disclosure, such as those in the CIITA and/or B2M genes can be introduced by methods known in the art. In certain embodiments, engineered nucleases may be used to introduce exogenous nucleic acid sequences for genetic modification of any cells referred to herein. Genome editing, or genome editing with engineered nucleases (GEEN) is a type of genetic engineering in which DNA is inserted, replaced, or removed from a genome using artificially engineered nucleases, or “molecular scissors.” The nucleases create specific double-stranded break (DSBs) at desired locations in the genome, and harness the cell's endogenous mechanisms to repair the induced break by natural processes of homologous recombination (HR) and nonhomologous end-joining (NHEJ). Non-limiting engineered nucleases include: Zinc finger nucleases (ZFNs), Transcription Activator-Like Effector Nucleases (TALENs), the CRISPR/Cas9 system, and engineered meganuclease re-engineered homing endonucleases. Any of the engineered nucleases known in the art can be used in certain aspects of the methods and compositions.

The engineered iNKT cells may be modified using methods that employ RNA interference. It is commonly practiced in genetic analysis that in order to understand the function of a gene or a protein function one interferes with it in a sequence-specific way and monitors its effects on the organism. However, in some organisms it is difficult or impossible to perform site-specific mutagenesis, and therefore more indirect methods have to be used, such as silencing the gene of interest by short RNA interference (siRNA). However, gene disruption by siRNA can be variable and incomplete. Genome editing with nucleases such as ZFN is different from siRNA in that the engineered nuclease is able to modify DNA-binding specificity and therefore can in principle cut any targeted position in the genome, and introduce modification of the endogenous sequences for genes that are impossible to specifically target by conventional RNAi. Furthermore, the specificity of ZFNs and TALENs are enhanced as two ZFNs are required in the recognition of their portion of the target and subsequently direct to the neighboring sequences.

Meganucleases may be employed to modify engineered iNKT cells. Meganucleases, found commonly in microbial species, have the unique property of having very long recognition sequences (>14 bp) thus making them naturally very specific. This can be exploited to make site-specific DSB in genome editing; however, the challenge is that not enough meganucleases are known, or may ever be known, to cover all possible target sequences. To overcome this challenge, mutagenesis and high throughput screening methods have been used to create meganuclease variants that recognize unique sequences. Others have been able to fuse various meganucleases and create hybrid enzymes that recognize a new sequence. Yet others have attempted to alter the DNA interacting aminoacids of the meganuclease to design sequence specific meganucelases in a method named rationally designed meganuclease (U.S. Pat. No. 8,021,867, incorporated herein by reference). Meganuclease have the benefit of causing less toxicity in cells compared to methods such as ZFNs likely because of more stringent DNA sequence recognition; however, the construction of sequence specific enzymes for all possible sequences is costly and time consuming as one is not benefiting from combinatorial possibilities that methods such as ZFNs and TALENs utilize. So there are both advantages and disadvantages.

As opposed to meganucleases, the concept behind ZFNs and TALENs is more based on a non-specific DNA cutting enzyme which would then be linked to specific DNA sequence recognizing peptides such as zinc fingers and transcription activator-like effectors (TALEs). One way was to find an endonuclease whose DNA recognition site and cleaving site were separate from each other, a situation that is not common among restriction enzymes. Once this enzyme was found, its cleaving portion could be separated which would be very non-specific as it would have no recognition ability. This portion could then be linked to sequence recognizing peptides that could lead to very high specificity. An example of a restriction enzyme with such properties is FokI. Additionally FokI has the advantage of requiring dimerization to have nuclease activity and this means the specificity increases dramatically as each nuclease partner would recognize a unique DNA sequence. To enhance this effect, FokI nucleases have been engineered that can only function as heterodimers and have increased catalytic activity. The heterodimer functioning nucleases would avoid the possibility of unwanted homodimer activity and thus increase specificity of the DSB.

Although the nuclease portion of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically happen in repeats that are 3 bp apart and are found in diverse combinations in a variety of nucleic acid interacting proteins such as transcription factors. TALES on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Zinc fingers have been more established in these terms and approaches such as modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries among other methods have been used to make site specific nucleases.

Thus, embodiments of the disclosure may or may not include the targeting of endogenous sequences to reduce or knock out expression of one or more certain endogenous sequences. In specific embodiments, disruption of one or more of the following genes may block the rearrangement of endogenous TCRs. To produce guide RNAs or siRNAs, for example, to target the noted genes below, their sequences are provided below as examples:

B-2 microglobin (B2M) (also known as IMD43) is located at 15q21.1 and has the
following mRNA sequence:
(SEQ ID NO: 66)
agtggaggcgtcgcgctggcgggcattcctgaagctgacagcattcgggccgagatgtctcgctccgtggccttagct
gtgctcgcgctactctctctttctggcctggaggctatccagcgtactccaaagattcaggtttactcacgtcatccagcagagaatggaa
agtcaaatttcctgaattgctatgtgtctgggtttcatccatccgacattgaagttgacttactgaagaatggagagagaattgaaaaagtg
gagcattcagacttgtctttcagcaaggactggtctttctatctcttgtactacactgaattcacccccactgaaaaagatgagtatgcctgc
cgtgtgaaccatgtgactttgtcacagcccaagatagttaagtggggtaagtcttacattcttttgtaagctgctgaaagttgtgtatgagta
gtcatatcataaagctgctttgatataaaaaaggtctatggccatactaccctgaatgagtcccatcccatctgatataaacaatctgcatat
tgggattgtcagggaatgttcttaaagatcagattagtggcacctgctgagatactgatgcacagcatggtttctgaaccagtagtttccct
gcagttgagcagggagcagcagcagcacttgcacaaatacatatacactcttaacacttcttacctactggcttcctctagcttttgtggc
agcttcaggtatatttagcactgaacgaacatctcaagaaggtataggcctagtttgtaagtcctgctgtcctagcatcctataatcctgga
cttctccagtactttctggctggattggtatctgaggctagtaggaagggcttgttcctgctgggtagctctaaacaatgtattcatgggta
ggaacagcagcctattctgccagccttatttctaaccattttagacatttgttagtacatggtattttaaaagtaaaacttaatgtcttccttttttt
tctccactgtattacatagatcgagacatgtaagcagcatcatggaggtaagtattgaccttgagaaaatgtttttgtttcactgtcctgag
gactatttatagacagctctaacatgataaccctcactatgtggagaacattgacagagtaacattttagcagggaaagaagaatcctac
agggtcatgttcccttctcctgtggagtggcatgaagaaggtgtatggccccaggtatggccatattactgaccctctacagagagggc
aaaggaactgccagtatggtattgcaggataaaggcaggtggttacccacattacctgcaaggctttgatctttcttctgccatttccacat
tggacatctctgctgaggagagaaaatgaaccactatacctttgtataatgagtatattcttcagacagaagagaggagttatacagct
ctgcagacatcccattcctgtatggggactgtgtttgcctcttagaggttcccaggccactagaggagataaagggaaacagattgttat
aacttgatataatgatactataatagatgtaactacaaggagctccagaagcaagagagagggaggaacttggacttctctgcatcttta
gttggagtccaaaggcttttcaatgaaattctactgcccagggtacattgatgctgaaaccccattcaaatctcctgttatattctagaacag
ggaattgatttgggagagcatcaggaaggtggatgatctgcccagtcacactgttagtaaattgtagagccaggacctgaactctaatat
agtcatgtgttacttaatgacggggacatgttctgagaaatgcttacacaaacctaggtgttgtagcctactacacgcataggctacatgg
tatagcctattgctcctagactacaaacctgtacagcctgaactgtactgaatactgtgggcagttgtaacacaatggtaagtatttgtgta
tctaaacatagaagttgcagtaaaaatatgctattttaatcttatgagaccactgtcatatatacagtccatcattgaccaaaacatcatatca
gcattttttcttctaagattttgggagcaccaaagggatacactaacaggatatactctttataatgggtttggagaactgtctgcagctactt
ctttttaaaaaggtgatctacacagtagaaattagacaagtaggtaatgagatctgcaatccaaataaaataaattcattgctaacctattat
ttcttttcaggtttgaagatgccgcatttggattggatgaattccaaattctgcttgcttgcttataatattgatatgcttatacacttacactttat
gcacaaaatgtagggttataataatgttaacatggacatgatcttctttataattctactttgagtgctgtctccatgtttgatgtatctgagca
ggttgctccacaggtagctctaggagggctggcaacttagaggtggggagcagagaattctcttatccaacatcaacatcttggtcaga
tttgaactcttcaatctcttgcactcaaagcttgttaagatagttaagcgtgcataagttaacttccaatttacatactctgcttagaatttggg
ggaaaatttagaaatataattgacaggattattggaaatttgttataatgaatgaaacattttgtcatataagattcatatttacttcttatacattt
gataaagtaaggcatggttgtggttaatctggtttatttttgttccacaagttaaataaatcataaaacttga
Human class II major histocompatibility complex transactivator (CIITA) gene is
located at 16p13.13 with an mRNA sequence:
(SEQ ID NO: 67)
ggttagtgatgaggctagtgatgaggctgtgtgcttctgagctgggcatccgaaggcatccttggggaagctgagggcacgaggagg
ggctgccagactccgggagctgctgcctggctgggattcctacacaatgcgttgcctggctccacgccctgctgggtcctacctgtca
gagccccaaggcagctcacagtgtgccaccatggagttggggcccctagaaggtggctacctggagcttcttaacagcgatgctgac
cccctgtgcctctaccacttctatgaccagatggacctggctggagaagaagagattgagctctactcagaacccgacacagacacca
tcaactgcgaccagttcagcaggctgttgtgtgacatggaaggtgatgaagagaccagggaggcttatgccaatatcgcggaactgg
accagtatgtcttccaggactcccagctggagggcctgagcaaggacattttcaagcacataggaccagatgaagtgatcggtgaga
gtatggagatgccagcagaagttgggcagaaaagtcagaaaagaccatcccagaggagcttccggcagacctgaagcactggaa
gccagctgagccccccactgtggtgactggcagtctcctagtgggaccagtgagcgactgctccaccctgccctgcctgccactgcct
gcgctgttcaaccaggagccagcctccggccagatgcgcctggagaaaaccgaccagattcccatgcctttctccagttcctcgttga
gctgcctgaatctccctgagggacccatccagtttgtccccaccatctccactctgccccatgggctctggcaaatctctgaggctggaa
caggggtctccagtatattcatctaccatggtgaggtgccccaggccagccaagtaccccctcccagtggattcactgtccacggcctc
ccaacatctccagaccggccaggctccaccagccccttcgctccatcagccactgacctgcccagcatgcctgaacctgccctgacct
cccgagcaaacatgacagagcacaagacgtcccccacccaatgcccggcagctggagaggtctccaacaagcttccaaaatggcct
gagccggtggagcagttctaccgctcactgcaggacacgtatggtgccgagcccgcaggcccggatggcatcctagtggaggtgg
atctggtgcaggccaggctggagaggagcagcagcaagagcctggagcgggaactggccaccccggactgggcagaacggcag
ctggcccaaggaggcctggctgaggtgctgttggctgccaaggagcaccggcggccgcgtgagacacgagtgattgctgtgctgg
gcaaagctggtcagggcaagagctattgggctggggcagtgagccgggcctgggcttgtggccggcttccccagtacgactttgtctt
ctctgtcccctgccattgcttgaaccgtccgggggatgcctatggcctgcaggatctgctcttctccctgggcccacagccactcgtgg
cggccgatgaggttttcagccacatcttgaagagacctgaccgcgttctgctcatcctagacggcttcgaggagctggaagcgcaaga
tggcttcctgcacagcacgtgcggaccggcaccggcggagccctgctccctccgggggctgctggccggccttttccagaagaagc
tgctccgaggttgcaccctcctcctcacagcccggccccggggccgcctggtccagagcctgagcaaggccgacgccctatttgag
ctgtccggcttctccatggagcaggcccaggcatacgtgatgcgctactttgagagctcagggatgacagagcaccaagacagagcc
ctgacgctcctccgggaccggccacttcttctcagtcacagccacagccctactttgtgccgggcagtgtgccagctctcagaggccct
gctggagcttggggaggacgccaagctgccctccacgctcacgggactctatgtcggcctgctgggccgtgcagccctcgacagcc
cccccggggccctggcagagctggccaagctggcctgggagctgggccgcagacatcaaagtaccctacaggaggaccagttcc
catccgcagacgtgaggacctgggcgatggccaaaggcttagtccaacacccaccgcgggccgcagagtccgagctggccttccc
cagcttcctcctgcaatgcttcctgggggccctgtggctggctctgagtggcgaaatcaaggacaaggagctcccgcagtacctagca
ttgaccccaaggaagaagaggccctatgacaactggctggagggcgtgccacgctttctggctgggctgatcttccagcctcccgcc
cgctgcctgggagccctactcgggccatcggcggctgcctcggtggacaggaagcagaaggtgcttgcgaggtacctgaagcggc
tgcagccggggacactgcgggcgcggcagctgctggagctgctgcactgcgcccacgaggccgaggaggctggaatttggcagc
acgtggtacaggagctccccggccgcctctcttttctgggcacccgcctcacgcctcctgatgcacatgtactgggcaaggccttgga
ggcggcgggccaagacttctccctggacctccgcagcactggcatttgcccctctggattggggagcctcgtgggactcagctgtgtc
acccgtttcagggctgccttgagcgacacggtggcgctgtgggagtccctgcagcagcatggggagaccaagctacttcaggcagc
agaggagaagttcaccatcgagcctttcaaagccaagtccctgaaggatgtggaagacctgggaaagcttgtgcagactcagaggac
gagaagttcctcggaagacacagctggggagctccctgctgttcgggacctaaagaaactggagtttgcgctgggccctgtctcagg
cccccaggctttccccaaactggtgcggatcctcacggccttttcctccctgcagcatctggacctggatgcgctgagtgagaacaaga
tcggggacgagggtgtctcgcagctctcagccaccttcccccagctgaagtccttggaaaccctcaatctgtcccagaacaacatcact
gacctgggtgcctacaaactcgccgaggccctgccttcgctcgctgcatccctgctcaggctaagcttgtacaataactgcatctgcga
cgtgggagccgagagcttggctcgtgtgcttccggacatggtgtccctccgggtgatggacgtccagtacaacaagttcacggctgcc
ggggcccagcagctcgctgccagccttcggaggtgtcctcatgtggagacgctggcgatgtggacgcccaccatcccattcagtgtc
caggaacacctgcaacaacaggattcacggatcagcctgagatgatcccagctgtgctctggacaggcatgttctctgaggacactaa
ccacgctggaccttgaactgggtacttgtggacacagctcttctccaggctgtatcccatgagcctcagcatcctggcacccggcccct
gctggttcagggttggcccctgcccggctgcggaatgaaccacatcttgctctgctgacagacacaggcccggctccaggctccttta
gcgcccagttgggtggatgcctggtggcagctgcggtccacccaggagccccgaggccttctctgaaggacattgcggacagccac
ggccaggccagagggagtgacagaggcagccccattctgcctgcccaggcccctgccaccctggggagaaagtacttctttttttttat
ttttagacagagtctcactgagcccaggctggcgtgcagtggtgcgatctgggttcactgcaacctccgcctcagggttcaagcgattc
ttctgcttcagcctcccgagtagctgggactacaggcacccaccatcatgtctggctaatttttcatttttagtagagacagggttttgccat
gttggccaggctggtctcaaactcttgacctcaggtgatccacccacctcagcctcccaaagtgctgggattacaagcgtgagccactg
caccgggccacagagaaagtacttctccaccctgctctccgaccagacaccttgacagggcacaccgggcactcagaagacactga
tgggcaacccccagcctgctaattccccagattgcaacaggctgggcttcagtggcagctgcttttgtctatgggactcaatgcactgac
attgttggccaaagccaaagctaggcctggccagatgcaccagcccttagcagggaaacagctaatgggacactaatggggcggtg
agaggggaacagactggaagcacagcttcatttcctgtgtcttttttcactacattataaatgtctctttaatgtcacaggcaggtccaggg
tttgagttcataccctgttaccattttggggtacccactgctctggttatctaatatgtaacaagccaccccaaatcatagtggcttaaaaca
acactcacattta.
Human T cell receptor alpha chain (TRAC) mRNA sequence is as follows:
(SEQ ID NO: 72)
ttttgaaacccttcaaaggcagagacttgtccagcctaacctgcctgctgctcctagctcctgaggctcagggcccttggcttctgtccgc
tctgctcagggccctccagcgtggccactgctcagccatgctcctgctgctcgtcccagtgctcgaggtgatttttaccctgggaggaa
ccagagcccagtcggtgacccagcttggcagccacgtctctgtctctgaaggagccctggttctgctgaggtgcaactactcatcgtct
gttccaccatatctcttctggtatgtgcaataccccaaccaaggactccagcttctcctgaagtacacatcagcggccaccctggttaaa
ggcatcaacggttttgaggctgaatttaagaagagtgaaacctccttccacctgacgaaaccctcagcccatatgagcgacgcggctg
agtacttctgtgctgtgagtgatctcgaaccgaacagcagtgcttccaagataatctttggatcagggaccagactcagcatccggcca
aatatccagaaccctgaccctgccgtgtaccagctgagagactctaaatccagtgacaagtctgtctgcctattcaccgattttgattctca
aacaaatgtgtcacaaagtaaggattctgatgtgtatatcacagacaaaactgtgctagacatgaggtctatggacttcaagagcaacag
tgctgtggcctggagcaacaaatctgactttgcatgtgcaaacgccttcaacaacagcattattccagaagacaccttcttccccagccc
agaaagttcctgtgatgtcaagctggtcgagaaaagctttgaaacagatacgaacctaaactttcaaaacctgtcagtgattgggttccg
aatcctcctcctgaaagtggccgggtttaatctgctcatgacgctgcggctgtggtccagctgagatctgcaagattgtaagacagcctg
tgctccctcgctccttcctctgcattgcccctcttctccctctccaaacagagggaactctcctacccccaaggaggtgaaagctgctacc
acctctgtgcccccccggtaatgccaccaactggatcctacccgaatttatgattaagattgctgaagagctgccaaacactgctgccac
cccctctgttcccttattgctgcttgtcactgcctgacattcacggcagaggcaaggctgctgcagcctcccctggctgtgcacattccct
cctgctccccagagactgcctccgccatcccacagatgatggatcttcagtgggttctcttgggctctaggtcctggagaatgttgtgag
gggtttatttttttttaatagtgttcataaagaaatacatagtattcttcttctcaagacgtggggggaaattatctcattatcgaggccctgcta
tgctgtgtgtctgggcgtgttgtatgtcctgctgccgatgccttcattaaaatgatttggaa.
Human T cell receptor beta chain (TRBC1) mRNA sequence is as follows:
(SEQ ID NO: 73)
tgcatcctagggacagcatagaaaggaggggcaaagtggagagagagcaacagacactgggatggtgaccccaaaacaatgagg
gcctagaatgacatagttgtgcttcattacggcccattcccagggctctctctcacacacacagagcccctaccagaaccagacagctc
tcagagcaaccctggctccaacccctcttccctttccagaggacctgaacaaggtgttcccacccgaggtcgctgtgtttgagccatca
gaagcagagatctcccacacccaaaaggccacactggtgtgcctggccacaggcttcttccccgaccacgtggagctgagctggtg
ggtgaatgggaaggaggtgcacagtggggtcagcacggacccgcagcccctcaaggagcagcccgccctcaatgactccagata
ctgcctgagcagccgcctgagggtctcggccaccttctggcagaacccccgcaaccacttccgctgtcaagtccagttctacgggctc
tcggagaatgacgagtggacccaggatagggccaaacccgtcacccagatcgtcagcgccgaggcctggggtagagcaggtgag
tggggcctggggagatgcctggaggagattaggtgagaccagctaccagggaaaatggaaagatccaggtagcagacaagactag
atccaaaaagaaaggaaccagcgcacaccatgaaggagaattgggcacctgtggttcattcactcccagattctcagcccaacagag
ccaagcagctgggtcccattctatgtggcctgtgtaactctcatctgggtggtgccccccatccccctcagtgctgccacatgccatgg
attgcaaggacaatgtggctgacatctgcatggcagaagaaaggaggtgctgggctgtcagaggaagctggtctgggcctgggagt
ctgtgccaactgcaaatctgactttacttttaattgcctatgaaaataaggtctctcatttattttcctctccctgctttctttcagactgtggcttt
acctcgggtaagtaagccatcctatcctctccctctctcatggacttgacctagaaccaaggcatgaagaactcacagacactggagg
gtggagggtgggagagaccagagctacctgtgcacaggtacccacctgtccacctccgtgccaacagtgtcctaccagcaaggggt
cctgtctgccaccatcctctatgagatcctgctagggaaggccaccctgtatgctgtgctggtcagcgcccttgtgttgatggccatggt
aagcaggagggcaggatggggccagcaggctggaggtgacacactgacaccaagcacccagaagtatagagtccctgccaggat
tggagctgggcagtagggagggaagagatttcattcaggtgcctcagaagataacttgcacctctgtaggatcacagtggaagggtca
tgctgggaaggagaagctggagtcaccagaaaacccaatggatgttgtgatgagccttactatttgtgtggtcaatgggccctactactt
tctctcaatcctcacaactcctggctcttaataacccccaaaactttctcttctgcaggtcaagagaaaggatttctgaaggcagccctgg
aagtggagttaggagcttctaacccgtcatggtttcaatacacattcacttagccagcgcttctgaagagctgactcacctctctgcatc
ccaatagatatccccctatgtgcatgcacacctgcacactcacggctgaaatctccctaacccagggggaccttagcatgcctaagtga
ctaaaccaataaaaatgttctggtctggcctgactctgacttgtgaatgtctggatagctccttggctgtctctgaactccctgtgactctcc
ccattcagtcaggatagaaacaagaggtattcaaggaaaatgcagactcttcacgtaagagggatgaggggcccaccttgagatcaat
agcag.
Human TRBC2 T cell receptor beta constant 2 (TCRB2) sequence is as follows:
(SEQ ID NO: 74)
atggcgtagtccccaaagaacgaggacctagtaacataattgtgcttcattatggtcctttcccggccttctctctcacacatacacagag
cccctaccaggaccagacagctctcagagcaaccctagccccattacctcttccctttccagaggacctgaaaaacgtgttcccaccc
gaggtcgctgtgtttgagccatcagaagcagagatctcccacacccaaaaggccacactggtgtgcctggccacaggcttctacccc
gaccacgtggagctgagctggtgggtgaatgggaaggaggtgcacagtggggtcagcacagacccgcagcccctcaaggagcag
cccgccctcaatgactccagatactgcctgagcagccgcctgagggtctcggccaccttctggcagaacccccgcaaccacttccgc
tgtcaagtccagttctacgggctctcggagaatgacgagtggacccaggatagggccaaacctgtcacccagatcgtcagcgccgag
gcctggggtagagcaggtgagtggggcctggggagatgcctggaggagattaggtgagaccagctaccagggaaaatggaaaga
tccaggtagcggacaagactagatccagaagaaagccagagtggacaaggtgggatgatcaaggttcacagggtcagcaaagcac
ggtgtgcacttcccccaccaagaagcatagaggctgaatggagcacctcaagctcattcttcatcagatcctgacaccttagagctaa
gctttcaagtctccctgaggaccagccatacagctcagcatctgagtggtgtgcatcccattctcttctggggtcctggtttcctaagatca
tagtgaccacttcgctggcactggagcagcatgagggagacagaaccagggctatcaaaggaggctgactttgtactatctgatatgc
atgtgtttgtggcctgtgagtctgtgatgtaaggctcaatgtccttacaaagcagcattctctcatccatttttcttcccctgttttctttcagact
gtggcttcacctccggtaagtgagtctctcctttttctctctatctttcgccgtctctgctctcgaaccagggcatggagaatccacggaca
caggggcgtgagggaggccagagccacctgtgcacaggtacctacatgctctgttcttgtcaacagagtcttaccagcaaggggtcct
gtctgccaccatcctctatgagatcttgctagggaaggccaccttgtatgccgtgctggtcagtgccctcgtgctgatggccatggtaag
gaggagggtgggatagggcagatgatgggggcaggggatggaacatcacacatgggcataaaggaatctcagagccagagcaca
gcctaatatatcctatcacctcaatgaaaccataatgaagccagactggggagaaaatgcagggaatatcacagaatgcatcatggga
ggatggagacaaccagcgagccctactcaaattaggcctcagagcccgcctcccctgccctactcctgctgtgccatagcccctgaa
accctgaaaatgttctctcttccacaggtcaagagaaaggattccagaggctagctccaaaaccatcccaggtcattcttcatcctcacc
caggattctcctgtacctgctcccaatctgtgacctaaaagtgattctcactctgatctcatctcctacttacatgaatacttctctattatct
gtttccctgaagattgagctcccaacccccaagtacgaaataggctaaaccaataaaaaattgtgtgttgggcctggttgcatttcagga
gtgtctgtggagttctgctcatcactgacctatcttctgatttagggaaagcagcattcgcttggacatctgaagtgacagccctctttctct
ccacccaatgctgctttctcctgttcatcctgatggaagtctcaacaca.

Inhibitory nucleic acids or any ways of inhibiting gene expression of CIITA and/or B2M known in the art are contemplated in certain embodiments. Examples of an inhibitory nucleic acid include but are not limited to siRNA (small interfering RNA), short hairpin RNA (shRNA), double-stranded RNA, an antisense oligonucleotide, a ribozyme and a nucleic acid encoding thereof. An inhibitory nucleic acid may inhibit the transcription of a gene or prevent the translation of a gene transcript in a cell. An inhibitory nucleic acid may be from 16 to 1000 nucleotides long, and in certain embodiments from 18 to 100 nucleotides long. The nucleic acid may have nucleotides of at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 50, 60, 70, 80, 90 or any range derivable therefrom. An siRNA naturally present in a living animal is not “isolated,” but a synthetic siRNA, or an siRNA partially or completely separated from the coexisting materials of its natural state is “isolated.” An isolated siRNA can exist in substantially purified form, or can exist in a non-native environment such as, for example, a cell into which the siRNA has been delivered.

Inhibitory nucleic acids are well known in the art. For example, siRNA and double-stranded RNA have been described in U.S. Pat. Nos. 6,506,559 and 6,573,099, as well as in U.S. Patent Publications 2003/0051263, 2003/0055020, 2004/0265839, 2002/0168707, 2003/0159161, and 2004/0064842, all of which are herein incorporated by reference in their entirety.

Particularly, an inhibitory nucleic acid may be capable of decreasing the expression of the protein or mRNA by at least 10%, 20%, 30%, or 40%, more particularly by at least 50%, 60%, or 70%, and most particularly by at least 75%, 80%, 90%, 95% or more or any range or value in between the foregoing.

In further embodiments, there are synthetic nucleic acids that are protein inhibitors. An inhibitor may be between 17 to 25 nucleotides in length and comprises a 5′ to 3′ sequence that is at least 90% complementary to the 5′ to 3′ sequence of a mature mRNA. In certain embodiments, an inhibitor molecule is 17, 18, 19, 20, 21, 22, 23, 24, or 25 nucleotides in length, or any range derivable therein. Moreover, an inhibitor molecule has a sequence (from 5′ to 3′) that is or is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature mRNA, particularly a mature, naturally occurring mRNA, such as a mRNA to B2M, CIITA, TRAC, TRBC1, or TRBC2. One of skill in the art could use a portion of the probe sequence that is complementary to the sequence of a mature mRNA as the sequence for an mRNA inhibitor. Moreover, that portion of the probe sequence can be altered so that it is still 90% complementary to the sequence of a mature mRNA.

In cases wherein the engineered iNKT cells comprise one or more suicide genes for subsequent depletion upon need, the suicide gene may be of any suitable kind. The iNKT cells of the disclosure may express a suicide gene product that may be enzyme-based, for example. Examples of suicide gene products include herpes simplex virus thymidine kinase (HSV-TK), purine nucleoside phosphorylase (PNP), cytosine deaminase (CD), carboxypetidase G2, cytochrome P450, linamarase, beta-lactamase, nitroreductase (NTR), carboxypeptidase A, or inducible caspase 9. Thus, in specific cases, the suicide gene may encode thymidine kinase (TK). In specific cases, the TK gene is a viral TK gene, such as a herpes simplex virus TK gene. In particular embodiments, the suicide gene product is activated by a substrate, such as ganciclovir, penciclovir, or a derivative thereof.

In specific embodiments, the suicide gene is sr39TK, and examples of corresponding sequences are as follows:

sr39TK cDNA sequence (codon-optimized): atgcctacactgctgcgggtgtacatcgatggccctcacggcatgggcaagaccacaaccacacagctgctggtggccctgggcag cagggacgatatcgtgtacgtgccagagcccatgacatattggcgcgtgctgggagcatccgagacaatcgccaacatctacaccac acagcacagactggatcagggagagatctccgccggcgacgcagcagtggtcatgaccagcgcccagatcacaatgggcatgcca tatgcagtgaccgacgccgtgctggcacctcacatcggaggagaggcaggctctagccacgcaccaccecctgccctgacaatcttt ctggatcggcaccctatcgccttcatgctgtgctacccagccgccagatatctgatgggcagcatgaccccacaggccgtgctggcct tcgtggccctgatcccacccaccctgccaggaacaaatatcgtgctgggcgccctgccagaggacaggcacatcgatagactggcc aagaggcagcgccccggagagcggctggacctggcaatgctggcagcaatcaggagagtgtacggcctgctggccaacaccgtg cggtatctgcagtgtggaggctcctggagagaggactggggacagctgtctggaacagcagtgcctccacagggagcagagccac agtccaatgcaggacctaggccacacatcggcgataccctgttcacactgtttcgcgcaccagagctgctggcacctaacggcgatct gtacaacgtgttcgcatgggcactggacgtgctggcaaagcggctgagatctatgcacgtgttcatcctggactacgaccagagccca gccggctgtagagatgccctgctgcagctgacaagcggcatggtgcagacccacgtgaccacacccggctctattccaacaatctgc gacctggctaggacctttgcaagagaaatgggcgaagctaactga (SEQ ID NO:70)

sr39TK amino acid sequence: MPTURVYIDGPHGMGKTTTTQLLVALGSRDDIVYVPEPMTYWRVLGASETIANIYT TQHRLDQGEISAGDAAVVMTSAQITMGMPYAVTDAVLAPHIGGEAGSSHAPPPALTI FLDRHPIAFMLCYPAARYLMGSMTPQAVLAFVALIPPTLPGTNIVLGALPEDRHIDRL AKRQRPGERLDLAMLAAIRRVYGLLANTVRYLQCGGSWREDWGQLSGTAVPPQGA EPQSNAGPRPHIGDTLFTLFRAPELLAPNGDLYNVFAWALDVLAKRLRSMHVFILDY DQSPAGCRDALLQLTSGMVQTHVTTPGSIPTICDLARTFAREMGEAN (SEQ ID NO:71).

In some embodiments, the engineered iNKT cells are able to be imaged or otherwise detected. In particular cases, the cells comprise an exogenous nucleic acid encoding a polypeptide that has a substrate that may be labeled for imaging, and the imaging may be fluorescent, radioactive, colorimetric, and so forth. In specific cases, the cells are detected by positron emission tomography. The cells in at least some cases express sr39TK gene that is a positron emission tomography (PET) reporter/thymidine kinase gene that allows for tracking of these genetically modified cells with PET imaging and elimination of these cells through the sr39TK suicide gene function.

Encompassed by the disclosure are populations of engineered iNKT cells. In particular aspects, iNKT clonal cells comprise an exogenous nucleic acid encoding an iNKT T-cell receptor (T-cell receptor) and lack surface expression of one or more HLA-I or HLA-II molecules. The iNKT cells may comprise an exogenous nucleic acid encoding a suicide gene, including an enzyme-based suicide gene such as thymidine kinase (TK). The TK gene may be a viral TK gene, such as a herpes simplex virus TK gene. In the cells of the population the suicide gene may be activated by a substrate, such as ganciclovir, penciclovir, or a derivative thereof, for example. The cells may comprise an exogenous nucleic acid encoding a polypeptide that has a substrate that may be labeled for imaging, and in some cases a suicide gene product is the polypeptide that has a substrate that may be labeled for imaging. In specific aspects, the suicide gene is sr39TK.

In certain embodiments of the iNKT cell population, the iNKT cells do not express surface HLA-I or -II molecules because of disrupted expression of genes encoding beta-2-microglobulin (B2M), major histocompatibility complex class II transactivator (CIITA), and/or HLA-I or HLA-II molecules, for example. The HLA-I or HLA-II molecules are not expressed on the cell surface of iNKT cells because the cells were manipulated by gene editing, in specific cases. The gene editing may or may not involve CRISPR-Cas9.

In particular cases for the iNKT cell population, the iNKT cells comprise nucleic acid sequences from a recombinant vector that was introduced into the cells, such as a viral vector (including at least a lentivirus, a retrovirus, an adeno-associated virus (AAV), a herpesvirus, or adenovirus).

In certain embodiments, the cells of the iNKT cell population may or may not have been exposed to, or are exposed to, one or more certain conditions. In certain cases, for example, the cells of the population not exposed or were not exposed to media that comprises animal serum. The cells of the population may or may not be frozen. In some cases the cells of the population are in a solution comprising dextrose, one or more electrolytes, albumin, dextran, and/or DMSO. The solution may comprise dextrose, one or more electrolytes, albumin, dextran, and DMSO. The cells may be in a solution that is sterile, nonpyogenic, and isotonic. In specific cases the iNKT cells have been activated, such as activated with alpha-galactosylceramide (α-GC). In specific aspects, the cell population comprises at least about 10²-10⁶ clonal cells. The cell population may comprise at least about 10⁶-10¹² total cells, in some cases.

In particular embodiments there is an invariant natural killer T (iNKT) cell population comprising: clonal iNKT cells comprising one or more exogenous nucleic acids encoding an iNKT T-cell receptor (T-cell receptor) and a thymidine kinase suicide, wherein the clonal iNKT cells have been engineered not to express functional beta-2-microglobulin (B2M), major histocompatibility complex class II transactivator (CIITA), and/or HLA-I and HLA-II molecules and wherein the cell population is at least about 10⁶-10¹² total cells and comprises at least about 10²-10⁶ clonal cells. In some cases the cells are frozen in a solution.

### III. Formulations and Culture of the Cells

In particular embodiments, the ^{U}HSC-iNKT cells and/or precursors thereto may be specifically formulated and/or they may be cultured in a particular medium (whether or not they are present in an in vitro ATO culture system) at any stage of a process of generating the ^{U}HSC-iNKT cells. The cells may be formulated in such a manner as to be suitable for delivery to a recipient without deleterious effects.

The medium in certain aspects can be prepared using a medium used for culturing animal cells as their basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco).

In further embodiments, the medium may be a serum-free medium that is suitable for cell development. For example, the medium may comprise B-27® supplement, xeno-free B-27® supplement (available at world wide web at thermofisher.com/us/en/home/technical-resources/media-formulation.250.html), NS21 supplement (Chen et al., J Neurosci Methods, 2008 Jun. 30; 171(2): 239-247, incorporated herein in its entirety), GS21™ supplement (available at world wide web at amsbio.com/B-27.aspx), or a combination thereof at a concentration effective for producing T cells from the 3D cell aggregate.

In certain embodiments, the medium may comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and/or T3 (triodo-I-thyronine).

In some embodiments, the medium further comprises vitamins. In some embodiments, the medium comprises 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. In some embodiments, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. In some embodiments, the vitamins include or consist essentially of biotin, DL alpha tocopherol acetate, DL alpha-tocopherol, vitamin A, or combinations or salts thereof. In some embodiments, the medium further comprises proteins. In some embodiments, the proteins comprise albumin or bovine serum albumin, a fraction of BSA, catalase, insulin, transferrin, superoxide dismutase, or combinations thereof. In some embodiments, the medium further comprises one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. In some embodiments, the medium comprises one or more of the following: a B-27® supplement, xeno-free B-27® supplement, GS21™ supplement, or combinations thereof. In some embodiments, the medium comprises or further comprises amino acids, monosaccharides, inorganic ions. In some embodiments, the amino acids comprise arginine, cystine, isoleucine, leucine, lysine, methionine, glutamine, phenylalanine, threonine, tryptophan, histidine, tyrosine, or valine, or combinations thereof. In some embodiments, the inorganic ions comprise sodium, potassium, calcium, magnesium, nitrogen, or phosphorus, or combinations or salts thereof. In some embodiments, the medium further comprises one or more of the following: molybdenum, vanadium, iron, zinc, selenium, copper, or manganese, or combinations thereof. In certain embodiments, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21™ supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese.

In further embodiments, the medium may comprise externally added ascorbic acid. The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, mg/ml, or any range derivable therein.

The medium used may be supplemented with at least one externally added cytokine at a concentration from about 0.1 ng/mL to about 500 ng/mL, more particularly 1 ng/mL to 100 ng/mL, or at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein. Suitable cytokines, include but are not limited to, FLT3 ligand (FLT3L), interleukin 7 (IL-7), stem cell factor (SCF), thrombopoietin (TPO), IL-2, IL-4, IL-6, IL-15, IL-21, TNF-alpha, TGF-beta, interferon-gamma, interferon-lambda, TSLP, thymopentin, pleotrophin, and/or midkine. Particularly, the culture medium may include at least one of FLT3L and IL-7. More particularly, the culture may include both FLT3L and IL-7.

Other culturing conditions can be appropriately defined. For example, the culturing temperature can be about 20 to 40° C., such as at least, at most, or about 20, 21, 22, 23, 24, 25, 26, 27, 28, 29, 30, 31, 32, 33, 34, 35, 36, 37, 38, 39, 40° C. (or any range derivable therein), though the temperature may be above or below these values. The CO₂ concentration can be about 1, 2, 3, 4, 5, 6, 7, 8, 9, or 10% (or any range derivable therein), such as about 2% to 10%, for example, about 2 to 5%, or any range derivable therein. The oxygen tension can be at least or about 1, 5, 8, 10, 20%, or any range derivable therein.

In specific embodiments, the allogeneic HSC-engineered HLA-negative iNKT cells are specifically formulated. They may or may not be formulated as a cell suspension. In specific cases they are formulated in a single dose form. They may be formulated for systemic or local administration. In some cases the cells are formulated for storage prior to use, and the cell formulation may comprise one or more cryopreservation agents, such as DMSO (for example, in 5% DMSO). The cell formulation may comprise albumin, including human albumin, with a specific formulation comprising 2.5% human albumin. The cells may be formulated specifically for intravenous administration; for example, they are formulated for intravenous administration over less than one hour. In particular embodiments the cells are in a formulated cell suspension that is stable at room temperature for 1, 2, 3, or 4 hours or more from time of thawing.

In some embodiments, the method further comprises priming the T cells. In some embodiments, the T cells are primed with antigen presenting cells. In some embodiments, the antigen presenting cells present tumor antigens.

In particular embodiments, the exogenous TCR of the ^{U}HSC-iNKT cells may be of any defined antigen specificity. In some embodiments, it can be selected based on absent or reduced alloreactivity to the intended recipient (examples include certain virus-specific TCRs, xeno-specific TCRs, or cancer-testis antigen-specific TCRs). In the example where the exogenous TCR is non-alloreactive, during T cell differentiation the exogenous TCR suppresses rearrangement and/or expression of endogenous TCR loci through a developmental process called allelic exclusion, resulting in T cells that express only the non-alloreactive exogenous TCR and are thus non-alloreactive. In some embodiments, the choice of exogenous TCR may not necessarily be defined based on lack of alloreactivity. In some embodiments, the endogenous TCR genes have been modified by genome editing so that they do not express a protein. Methods of gene editing such as methods using the CRISPR/Cas9 system are known in the art and described herein.

In some embodiments, the isolated ^{U}HSC-iNKT cell or population thereof comprise a one or more chimeric antigen receptors (CARs). Examples of tumor cell antigens to which a CAR may be directed include at least 5T4, 8H9, αᵥβ₆ integrin, BCMA, B7-H3, B7-H6, CAIX, CA9, CD19, CD20, CD22, CD30, CD33, CD38, CD44, CD44v6, CD44v7/8, CD70, CD123, CD138, CD171, CEA, CSPG4, EGFR, EGFR family including ErbB2 (HER2), EGFRvIII, EGP2, EGP40, ERBB3, ERBB4, ErbB3/4, EPCAM, EphA2, EpCAM, folate receptor-a, FAP, FBP, fetal AchR, FRα, GD2, G250/CAIX, GD3, Glypican-3 (GPC3), Her2, IL-13Rα2, Lambda, Lewis-Y, Kappa, KDR, MAGE, MCSP, Mesothelin, Muc1, Muc16, NCAM, NKG2D Ligands, NY-ESO-1, PRAME, PSC1, PSCA, PSMA, ROR1, SP17, Survivin, TAG72, TEMs, carcinoembryonic antigen, HMW-MAA, AFP, CA-125, ETA, Tyrosinase, MAGE, laminin receptor, HPV E6, E7, BING-4, Calcium-activated chloride channel 2, Cyclin-B1, 9D7, EphA3, Telomerase, SAP-1, BAGE family, CAGE family, GAGE family, MAGE family, SAGE family, XAGE family, NY-ESO-1/LAGE-1, PAME, SSX-2, Melan-A/MART-1, GP100/pme117, TRP-1/-2, P. polypeptide, MC1R, Prostate-specific antigen, β-catenin, BRCA1/2, CML66, Fibronectin, MART-2, TGF-βRII, or VEGF receptors (e.g., VEGFR2), for example. The CAR may be a first, second, third, or more generation CAR. The CAR may be bispecific for any two nonidentical antigens, or it may be specific for more than two nonidentical antigens.

### IV. Additional Modifications and Polypeptide Embodiments

Additionally, the polypeptides of the disclosure may be chemically modified. Glycosylation of the polypeptides can be altered, for example, by modifying one or more sites of glycosylation within the polypeptide sequence to increase the affinity of the polypeptide for antigen (U.S. Pat. Nos. 5,714,350 and 6,350,861).

It is contemplated that a region or fragment of a polypeptide of the disclosure or a nucleic acid of the disclosure encoding for a polypeptide that may have an amino acid sequence that has, has at least or has at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 or more amino acid substitutions, contiguous amino acid additions, or contiguous amino acid deletions with respect to any of SEQ ID NOS: 20, 23, 26, 29, 32, 35, 38, 41, 44, 47, 50, 53, 56, 59, 62, 65, or 71 or with respect to the polypeptide encoded by any of SEQ ID NOS:1-19, 21, 22, 24, 25, 27, 28, 30, 31, 33, 34, 36, 37, 39, 40, 42, 43, 45, 46, 48, 49, 51, 52, 54, 55, 57, 58, 60, 61, 63, 64, 66-70, or 72-74.

Alternatively, a region or fragment of a polypeptide of the disclosure may have an amino acid sequence that comprises or consists of an amino acid sequence that is, is at least, or is at most 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% (or any range derivable therein) identical to any of SEQ ID NOS:20, 23, 26, 29, 32, 35, 38, 41, 44, 47, 50, 53, 56, 59, 62, 65, or 71 or to the polypeptide encoded by any of SEQ ID NOS:1-19, 21, 22, 24, 25, 27, 28, 30, 31, 33, 34, 36, 37, 39, 40, 42, 43, 45, 46, 48, 49, 51, 52, 54, 55, 57, 58, 60, 61, 63, 64, 66-70, or 72-74. Moreover, in some embodiments, a region or fragment comprises an amino acid region of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 or more contiguous amino acids starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 in any of SEQ ID NOS: 20, 23, 26, 29, 32, 35, 38, 41, 44, 47, 50, 53, 56, 59, 62, 65, or 71 or of the polypeptide encoded by any of SEQ ID NOS:1-19, 21, 22, 24, 25, 27, 28, 30, 31, 33, 34, 36, 37, 39, 40, 42, 43, 45, 46, 48, 49, 51, 52, 54, 55, 57, 58, 60, 61, 63, 64, 66-70, or 72-74. (where position 1 is at the N-terminus of the SEQ ID NO or the N terminus of the polypeptide encoded by the SEQ ID NO). The polypeptides of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000, 1500, or 2000 or more contiguous amino acids or nucleic acids, or any range derivable therein, of any of SEQ ID NOS: 20, 23, 26, 29, 32, 35, 38, 41, 44, 47, 50, 53, 56, 59, 62, 65, or 71 or of the polypeptide encoded by any of SEQ ID NOS:1-19, 21, 22, 24, 25, 27, 28, 30, 31, 33, 34, 36, 37, 39, 40, 42, 43, 45, 46, 48, 49, 51, 52, 54, 55, 57, 58, 60, 61, 63, 64, 66-70, or 72-74.

The polypeptides of the disclosure may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 substitutions (or any range derivable therein).

The substitution may be at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 650, 700, 750, 800, 850, 900, 1000, 1500, or 2000 (or any derivable range therein) of any of SEQ ID NOS: 20, 23, 26, 29, 32, 35, 38, 41, 44, 47, 50, 53, 56, 59, 62, 65, or 71 or of the polypeptide encoded by any of SEQ ID NOS:1-19, 21, 22, 24, 25, 27, 28, 30, 31, 33, 34, 36, 37, 39, 40, 42, 43, 45, 46, 48, 49, 51, 52, 54, 55, 57, 58, 60, 61, 63, 64, 66-70, or 72-74.

The polypeptides described herein may be of a fixed length of at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more amino acids (or any derivable range therein) of SEQ ID NOS: 20, 23, 26, 29, 32, 35, 38, 41, 44, 47, 50, 53, 56, 59, 62, 65, or 71 or of the polypeptide encoded by any of SEQ ID NOS:1-19, 21, 22, 24, 25, 27, 28, 30, 31, 33, 34, 36, 37, 39, 40, 42, 43, 45, 46, 48, 49, 51, 52, 54, 55, 57, 58, 60, 61, 63, 64, 66-70, or 72-74.

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins may be recombinant, or synthesized in vitro. Alternatively, a non-recombinant or recombinant protein may be isolated from bacteria. It is also contemplated that bacteria containing such a variant may be implemented in compositions and methods. Consequently, a protein need not be isolated.

The term “functionally equivalent codon” is used herein to refer to codons that encode the same amino acid, such as the six codons for arginine or serine, and also refers to codons that encode biologically equivalent amino acids.

It also will be understood that amino acid and nucleic acid sequences may include additional residues, such as additional N- or C-terminal amino acids, or 5′ or 3′ sequences, respectively, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

The following is a discussion based upon changing of the amino acids of a protein to create an equivalent, or even an improved, second-generation molecule. For example, certain amino acids may be substituted for other amino acids in a protein structure without appreciable loss of interactive binding capacity. Structures such as, for example, an enzymatic catalytic domain or interaction components may have amino acid substituted to maintain such function. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity.

In other embodiments, alteration of the function of a polypeptide is intended by introducing one or more substitutions. For example, certain amino acids may be substituted for other amino acids in a protein structure with the intent to modify the interactive binding capacity of interaction components. Structures such as, for example, protein interaction domains, nucleic acid interaction domains, and catalytic sites may have amino acids substituted to alter such function. Since it is the interactive capacity and nature of a protein that defines that protein's biological functional activity, certain amino acid substitutions can be made in a protein sequence, and in its underlying DNA coding sequence, and nevertheless produce a protein with different properties. It is thus contemplated by the inventors that various changes may be made in the DNA sequences of genes with appreciable alteration of their biological utility or activity.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. It is understood that an amino acid can be substituted for another having a similar hydrophilicity value and still produce a biologically equivalent and immunologically equivalent protein.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known and include: arginine and lysine; glutamate and aspartate; serine and threonine; glutamine and asparagine; and valine, leucine and isoleucine.

In specific embodiments, all or part of proteins described herein can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Alternatively, recombinant DNA technology may be employed wherein a nucleotide sequence that encodes a peptide or polypeptide is inserted into an expression vector, transformed or transfected into an appropriate host cell and cultivated under conditions suitable for expression.

One embodiment includes the use of gene transfer to cells, including microorganisms, for the production and/or presentation of proteins. The gene for the protein of interest may be transferred into appropriate host cells followed by culture of cells under the appropriate conditions. A nucleic acid encoding virtually any polypeptide may be employed. The generation of recombinant expression vectors, and the elements included therein, are discussed herein. Alternatively, the protein to be produced may be an endogenous protein normally synthesized by the cell used for protein production.

### V. Methods of Producing the ^{U}HSC-iNKT Cells

^{U}HSC-iNKT cells may be produced by any suitable method(s). The method(s) may utilize one or more successive steps for one or more modifications to cells and/or utilize one or more simultaneous steps for one or more modifications to cells. In specific embodiments, a starting source of cells are modified to become functional as iNKT cells followed by one or more steps to add one or more additional characteristics to the cells, such as the ability to be imaged, and/or the ability to be selectively killed, and/or the ability to be able to be used allogeneically. In specific embodiments, at least part of the process for generating ^{U}HSC-iNKT cells occurs in a specific in vitro culture system. An example of a specific in vitro culture system is one that allows differentiation of certain cells at high efficiency and high yield. In specific embodiments the in vitro culture system is an artificial thymic organoid (ATO) system.

In specific cases, ^{U}HSC-iNKT cells may be generated by the following: 1) genetic modification of donor HSCs to express iNKT TCRs (for example, via lentiviral vectors) and to eliminate expression of HLA-I/II molecules (for example, via CRISPR/Cas9-based gene editing); 2) in vitro differentiation into iNKT cells via an ATO culture, 3) in vitro iNKT cell purification and expansion, and 4) formulation and cryopreservation and/or use.

Particular embodiments of the disclosure provide methods of preparing a population of clonal invariant natural killer T (iNKT) cells comprising: a) selecting CD34+ cells from human peripheral blood cells (PBMCs); b) introducing one or more nucleic acids encoding a human iNKT T-cell receptor (TCR); c) eliminating expression of one or more HLA-I/II genes in the isolated human CD34+ cells; and, d) culturing isolated CD34+ cells expressing iNKT TCR in an artificial thymic organoid (ATO) system to produce iNKT cells, wherein the ATO system comprises a 3D cell aggregate comprising a selected population of stromal cells that express a Notch ligand and a serum-free medium. The method may further comprise isolating CD34− cells. In alternative embodiments, other culture systems than the ATO system is employed, such as a 2-D culture system or other forms of 3-D culture systems (e.g., FTOC-like culture, metrigel-aided culture).

Specific aspects of the disclosure relate to a novel three dimensional cell culture system to produce iNKT cells from less differentiated cells such as embryonic stem cells, pluripotent stem cells, hematopoietic stem or progenitor cells, induced pluripotent stem (iPS) cells, or stem or progenitor cells. Stem cells of any type may be utilized from various resources, including at least fetal liver, cord blood, and peripheral blood CD34+ cells (either G-CSF-mobilized or non-G-CSF-mobilized), for example.

In particular embodiments, the system involves using serum-free medium. In certain aspects, the system uses a serum-free medium that is suitable for cell development for culturing of a three-dimensional cell aggregate. Such a system produces sufficient amounts of ^{U}HSC-iNKT cells. In embodiments of the disclosure, the 3D cell aggregate is cultured in a serum-free medium comprising insulin for a time period sufficient for the in vitro differentiation of stem or progenitor cells to ^{U}HSC-iNKT cells or precursors to ^{U}HSC-iNKT cells.

Embodiments of a cell culture composition comprise an ATO 3D culture that uses highly-standardized, serum-free components and a stromal cell line to facilitate robust and highly reproducible T cell differentiation from human HSCs. In certain embodiments, cell differentiation in ATOs closely mimicked endogenous thymopoiesis and, in contrast to monolayer co-cultures, supported efficient positive selection of functional ^{U}HSC-iNKT. Certain aspects of the 3D culture compositions use serum-free conditions, avoid the use of human thymic tissue or proprietary scaffold materials, and facilitate positive selection and robust generation of fully functional, mature human ^{U}HSC-iNKT cells from source cells.

In particular embodiments, this ATO 3D culture system may comprise the aggregation in a 3D structure of human HSC with stromal cells expressing a Notch ligand, in the presence of an optimized medium containing FLT3 ligand (FLT3L), interleukin 7 (IL-7), B27, and ascorbic acid. Conditions that permit culture at the air-fluid interface may also be present. It has been determined that combinatorial signaling within ATOs from soluble factors (cytokines, ascorbic acid, B27 components, and stromal cell-derived factors) together with 3D cell-cell interactions between hematopoietic and stromal cells, facilitates human T lineage commitment, positive selection, and efficient differentiation into functional, mature T cells.

In particular embodiments, the 3D cell aggregate is created by mixing CD34+ transduced cells with the selected population of stromal cells on a physical matrix or scaffold. The method may further comprise centrifuging the CD34+ transduced cells and stromal cells to form a cell pellet that is placed on the physical matrix or scaffold. The Notch ligand expressed by the stromal cells may be intact, partial, or modified DLL1, DLL4, JAG1, JAG2, or a combination thereof. In specific cases, the Notch ligand is a human Notch ligand, such as human DLL1, for example.

The ATO system utilized to produce the iNKT cells may have a certain ratio of stromal cells to CD34+ cells. In specific cases, the ratio between stromal cells and CD34+ cells is about 1:5 to 1:20. The stromal cells may be a murine stromal cell line, a human stromal cell line, a selected population of primary stromal cells, a selected population of stromal cells differentiated from pluripotent stem cells in vitro, or a combination thereof. The stroma cells may be a selected population of stromal cells differentiated from hematopoietic stem or progenitor cells in vitro.

In methods of preparing a population of clonal iNKT cells, selecting iNKT cells lacking surface expression of HLA-I and HLA-II molecules may comprise contacting the iNKT cells with magnetic beads that bind to and positively select for iNKT cells and negatively select for HLA-I/II-negative cells. In specific embodiments, the magnetic beads are coated with monoclonal antibodies recognizing human iNKT TCRs, HLA-I molecules, or HLA-II molecules. In particular embodiments, the monoclonal antibodies are Clone 6B11 (recognizing human TCR Vα24-Jα18 thus recognizing human iNKT invariant TCR alpha chain), Clone 2M2 (recognizing human B2M thus recognizing cell surface-displayed human HLA-I molecules), Clone W6/32 (recognizing HLA-A,B,C thus recognizing human HLA-I molecules), and Clone Tü39 (recognizing human HLA-DR, DP, DQ thus recognizing human HLA-II molecules).

Cells produced by the preparation methods may be frozen. The produced cells may be in a solution comprising dextrose, one or more electrolytes, albumin, dextran, and DMSO. The solution may be sterile, nonpyogenic, and isotonic.

In particular embodiments, the ATO system utilizes feeder cells that may comprise CD34⁻ cells.

Preparation methods may further comprise activating and expanding the selected iNKT cells; for example, the selected iNKT cells have been activated with alpha-galactosylceramide (α-GC). The feeder cells may have been pulsed with α-GC.

Preparation methods of the disclosure may produce a population of clonal iNKT cells comprising at least about 10²-10⁶ clonal iNKT cells. The method may produce a cell population comprising at least about 10⁶-10¹² total cells. The produced cell population may be frozen and then thawed. In some cases of the preparation method, the method further comprises introducing one or more additional nucleic acids into the frozen and thawed cell population, such as the one or more additional nucleic acids encoding one or more therapeutic gene products, for example.

In specific embodiments, there may be provided a method of a 3D culture composition (e.g., ATO production), as developed, involves aggregation of the MS-5 murine stromal cell line transduced with human DLL1 (MS5-hDLL1, hereafter) with CD34⁺ HSPCs isolated from human cord blood, bone marrow, or G-CSF mobilized peripheral blood. Up to 1×10⁶ HSPCs are mixed with MS5-hDLL1 cells at an optimized ratio (typically 1:10 HSPCs to stromal cells).

For example, aggregation is achieved by centrifugation of the mixed cell suspension (“compaction aggregation”) followed by aspiration of the cell-free supernatant. In particular embodiments, the cell pellet may then be aspirated as a slurry in 5-10 ul of a differentiation medium and transferred as a droplet onto 0.4 urn nylon transwell culture inserts, which are floated in a well of differentiation medium, allowing the bottom of the insert to be in contact with medium and the top with air.

For example, the differentiation medium may comprise RPMI-1640, 5 ng/ml human FLT3L, 5 ng/ml human IL-7, 4% Serum-Free B27 Supplement, and 30 uM L-ascorbic acid. Medium may be completely replaced every 3-4 days from around the culture inserts. During the first 2 weeks of culture, cell aggregates may self-organize as ATOs, and early T cell lineage commitment and differentiation occurs. In certain aspects, ATOs are cultured for at least 6 weeks to allow for optimal T cell differentiation. Retrieval of hematopoietic cells from ATOs is achieved by disaggregating ATOs by pipetting.

Variations in the protocol permit the use of alternative components with varying impact on efficacy, specifically:

Base medium RPMI may be substituted for several commercially available alternatives (e.g. IMDM)

The stromal cell line used is MS-5, a previously described murine bone marrow cell line (Itoh et al, 1989), however MS-5 may be substituted for similar murine stromal cell lines (e.g. OP9, S17), human stromal cell lines (e.g. HS-5, HS-27a), primary human stromal cells, or human pluripotent stem cell-derived stromal cells.

The stromal cell line is transduced with a lentivirus encoding human DLL1 cDNA; however the method of gene delivery, as well as the Notch ligand gene, may be varied. Alternative Notch ligand genes include DLL4, JAG1, JAG2, and others. Notch ligands also include those described in U.S. Pat. Nos. 7,795,404 and 8,377,886, which are herein incorporated by reference. Notch ligands further include Delta 1, 3, and 4 and Jagged 1, 2.

The type and source of HSCs may include bone marrow, cord blood, peripheral blood, thymus, or other primary sources; or HSCs derived from human embryonic stem cells (ESC) or induced pluripotent stem cells (iPSC).

Cytokine conditions can be varied: e.g. levels of FLT3L and IL-7 may be changed to alter T cell differentiation kinetics; other hematopoietic cytokines such as Stem Cell Factor (SCF/KIT ligand), thrombopoietin (TPO), IL-2, IL-15 may be added.

Genetic modification may also be introduced to certain components to generate antigen-specific T cells, and to model positive and negative selection. Examples of these modifications include: transduction of HSCs with a lentiviral vector encoding an antigen-specific T cell receptor (TCR) or chimeric antigen receptor (CAR) for the generation of antigen-specific, allelically excluded naïve T cells; transduction of HSCs with gene/s to direct lineage commitment to specialized lymphoid cells. For example, transduction of HSCs with an invariant natural killer T cell (iNKT) associated TCR to generate functional iNKT cells in ATOs; transduction of the ATO stromal cell line (e.g., MS5-hDLL1) with human MHC genes (e.g. human CD1d gene) to enhance positive selection and maturation of both TCR engineered or non-engineered T cells in ATOs; and/or transduction of the ATO stromal cell line with an antigen plus costimulatory molecules or cytokines to enhance the positive selection of CAR T cells in ATOs.

In producing the engineered iNKT cells, CD34+ cells from human peripheral blood cells (PBMCs) may be modified by introducing certain exogenous gene(s) and by knocking out certain endogenous gene(s). The methods may further comprise culturing selected CD34+ cells in media prior to introducing one or more nucleic acids into the cells. The culturing may comprise incubating the selected CD34+ cells with medium comprising one or more growth factors, in some cases, and the one or more growth factors may comprise c-kit ligand, flt-3 ligand, and/or human thrombopoietin (TPO), for example. The growth factors may or may not be at a certain concentration, such as between about 5 ng/ml to about 500 ng/ml/.

In particular methods the nucleic acid(s) to be introduced into the cells are one or more nucleic acids that comprise a nucleic acid sequence encoding an α-TCR and a β-TCR. The methods may further comprise introducing into the selected CD34+ cells a nucleic acid encoding a suicide gene. In specific aspects, one nucleic acid encodes both the α-TCR and the β-TCR, or one nucleic acid encodes the α-TCR, the β-TCR, and the suicide gene. The suicide gene may be enzyme-based, such as thymidine kinase (TK) including a viral TK gene such as one from herpes simplex virus TK gene. The suicide gene may be activated by a substrate, such as ganciclovir, penciclovir, or a derivative thereof. The cells may be engineered to comprise an exogenous nucleic acid encoding a polypeptide that has a substrate that may be labeled for imaging. In some cases, a suicide gene product is a polypeptide that has a substrate that may be labeled for imaging, such as sr39TK.

The cells may be engineered to lack surface expression of HLA-I and/or HLA-II molecules, for example by discrupting the functional expression of genes encoding beta-2-microglobulin (B2M), major histocompatibility complex class II transactivator (CIITA), and/or HLA-I and HLA-II molecules. In the production methods, eliminating surface expression of one or more HLA-I/II molecules in the isolated human CD34+ cells may comprise introducing CRISPR and one or more guide RNAs (gRNAs) corresponding to B2M, CIITA, or individual HLA-I or HLA-II molecules into the cells. CRISPR or the one or more gRNAs are transfected into the cell by electroporation or lipid-mediated transfection in some cases. In specific embodiments, the nucleic acid encoding the TCR receptor is introduced into the cell using a recombinant vector such as a viral vector including at least a lentivirus, a retrovirus, an adeno-associated virus (AAV), a herpesvirus, or adenovirus, for example.

In manufacturing the engineered iNKT cells, the cells may be present in a particular serum-free medium, including one that comprises externally added ascorbic acid. In specific aspects, the serum-free medium further comprises externally added FLT3 ligand (FLT3L), interleukin 7 (IL-7), stem cell factor (SCF), thrombopoietin (TPO), stem cell factor (SCF), thrombopoietin (TPO), IL-2, IL-4, IL-6, IL-15, IL-21, TNF-alpha, TGF-beta, interferon-gamma, interferon-lambda, TSLP, thymopentin, pleotrophin, midkine, or combinations thereof. The serum-free medium may further comprise vitamins, including biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or combinations thereof or salts thereof. The serum-free medium may further comprise one or more externally added (or not) proteins, such as albumin or bovine serum albumin, a fraction of BSA, catalase, insulin, transferrin, superoxide dismutase, or combinations thereof. The serum-free medium may further comprise corticosterone, D-Galactose, ethanolamine, glutathione, L-carnitine, linoleic acid, linolenic acid, progesterone, putrescine, sodium selenite, or triodo-I-thyronine, or combinations thereof. The serum-free medium may comprise a B-27® supplement, xeno-free B-27® supplement, GS21™ supplement, or combinations thereof. Amino acids (including arginine, cysteine, isoleucine, leucine, lysine, methionine, glutamine, phenylalanine, threonine, tryptophan, histidine, tyrosine, or valine, or combinations thereof), monosaccharides, and/or inorganic ions (including sodium, potassium, calcium, magnesium, nitrogen, or phosphorus, or combinations or salts thereof, for example) may be present in the serum-free medium. The serum-free medium may further comprise molybdenum, vanadium, iron, zinc, selenium, copper, or manganese, or combinations thereof.

Cell culture conditions may be provided for the culture of 3D cell aggregates described herein and for the production of T cells and/or positive/negative selection thereof. In certain aspects, starting cells of a selected population may comprise at least or about 10⁴, 10⁵, 10⁶, 10⁷, 10⁸, 10⁹, 10¹⁰, 10¹¹, 10¹², 10¹³ cells or any range derivable therein. The starting cell population may have a seeding density of at least or about 10, 10¹, 10², 10³, 10⁴, 10⁵, 10⁶, 10⁷, 10⁸ cells/ml, or any range derivable therein.

A culture vessel used for culturing the 3D cell aggregates or progeny cells thereof can include, but is particularly not limited to: flask, flask for tissue culture, dish, petri dish, dish for tissue culture, multi dish, micro plate, micro-well plate, multi plate, multi-well plate, micro slide, chamber slide, tube, tray, CellSTACK® Chambers, culture bag, and roller bottle, as long as it is capable of culturing the stem cells therein. The stem cells may be cultured in a volume of at least or about 0.2, 0.5, 1, 2, 5, 10, 20, 30, 40, 50 ml, 100 ml, 150 ml, 200 ml, 250 ml, 300 ml, 350 ml, 400 ml, 450 ml, 500 ml, 550 ml, 600 ml, 800 ml, 1000 ml, 1500 ml, or any range derivable therein, depending on the needs of the culture. In a certain embodiment, the culture vessel may be a bioreactor, which may refer to any device or system that supports a biologically active environment. The bioreactor may have a volume of at least or about 2, 4, 5, 6, 8, 10, 15, 20, 25, 50, 75, 100, 150, 200, 500 liters, 1, 2, 4, 6, 8, 10, 15 cubic meters, or any range derivable therein.

The culture vessel can be cellular adhesive or non-adhesive and selected depending on the purpose. The cellular adhesive culture vessel can be coated with any of substrates for cell adhesion such as extracellular matrix (ECM) to improve the adhesiveness of the vessel surface to the cells. The substrate for cell adhesion can be any material intended to attach stem cells or feeder cells (if used). The substrate for cell adhesion includes collagen, gelatin, poly-L-lysine, poly-D-lysine, laminin, and fibronectin and mixtures thereof for example Matrigel™, and lysed cell membrane preparations.

Various defined matrix components may be used in the culturing methods or compositions. For example, recombinant collagen IV, fibronectin, laminin, and vitronectin in combination may be used to coat a culturing surface as a means of providing a solid support for pluripotent cell growth, as described in Ludwig et al. (2006a; 2006b), which are incorporated by reference in its entirety.

A matrix composition may be immobilized on a surface to provide support for cells. The matrix composition may include one or more extracellular matrix (ECM) proteins and an aqueous solvent. The term “extracellular matrix” is recognized in the art. Its components include one or more of the following proteins: fibronectin, laminin, vitronectin, tenascin, entactin, thrombospondin, elastin, gelatin, collagen, fibrillin, merosin, anchorin, chondronectin, link protein, bone sialoprotein, osteocalcin, osteopontin, epinectin, hyaluronectin, undulin, epiligrin, and kalinin Other extracellular matrix proteins are described in Kleinman et al., (1993), herein incorporated by reference. It is intended that the term “extracellular matrix” encompass a presently unknown extracellular matrix that may be discovered in the future, since its characterization as an extracellular matrix will be readily determinable by persons skilled in the art.

In some aspects, the total protein concentration in the matrix composition may be about 1 ng/mL to about 1 mg/mL. In some embodiments, the total protein concentration in the matrix composition is about 1 μg/mL to about 300 μg/mL. In more preferred embodiments, the total protein concentration in the matrix composition is about 5 μg/mL to about 200 μg/mL.

The extracellular matrix (ECM) proteins may be of natural origin and purified from human or animal tissues. Alternatively, the ECM proteins may be genetically engineered recombinant proteins or synthetic in nature. The ECM proteins may be a whole protein or in the form of peptide fragments, native or engineered. Examples of ECM protein that may be useful in the matrix for cell culture include laminin, collagen I, collagen IV, fibronectin and vitronectin. In some embodiments, the matrix composition includes synthetically generated peptide fragments of fibronectin or recombinant fibronectin.

In still further embodiments, the matrix composition includes a mixture of at least fibronectin and vitronectin. In some other embodiments, the matrix composition preferably includes laminin.

The matrix composition preferably includes a single type of extracellular matrix protein. In some embodiments, the matrix composition includes fibronectin, particularly for use with culturing progenitor cells. For example, a suitable matrix composition may be prepared by diluting human fibronectin, such as human fibronectin sold by Becton, Dickinson & Co. of Franklin Lakes, N.J. (BD) (Cat#354008), in Dulbecco's phosphate buffered saline (DPBS) to a protein concentration of 5 μg/mL to about 200 μg/mL. In a particular example, the matrix composition includes a fibronectin fragment, such as RetroNectin®. RetroNectin® is a ˜63 kDa protein of (574 amino acids) that contains a central cell-binding domain (type III repeat, 8, 9, 10), a high affinity heparin-binding domain II (type III repeat, 12, 13, 14), and CS1 site within the alternatively spliced IIICS region of human fibronectin.

In some other embodiments, the matrix composition may include laminin. For example, a suitable matrix composition may be prepared by diluting laminin (Sigma-Aldrich (St. Louis, Mo.); Cat#L6274 and L2020) in Dulbecco's phosphate buffered saline (DPBS) to a protein concentration of 5 μg/ml to about 200 μg/ml.

In some embodiments, the matrix composition is xeno-free, in that the matrix is or its component proteins are only of human origin. This may be desired for certain research applications. For example in the xeno-free matrix to culture human cells, matrix components of human origin may be used, wherein any non-human animal components may be excluded. In certain aspects, Matrigel™ may be excluded as a substrate from the culturing composition. Matrigel™ is a gelatinous protein mixture secreted by mouse tumor cells and is commercially available from BD Biosciences (New Jersey, USA). This mixture resembles the complex extracellular environment found in many tissues and is used frequently by cell biologists as a substrate for cell culture, but it may introduce undesired xeno antigens or contaminants.

In certain embodiments, cells containing an exogenous nucleic acid may be identified in vitro or in vivo by including a marker in the expression vector or the exogenous nucleic acid. Such markers would confer an identifiable change to the cell permitting easy identification of cells containing the expression vector. Generally, a selection marker may be one that confers a property that allows for selection. A positive selection marker may be one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

Selectable markers may include a type of reporter gene used in laboratory microbiology, molecular biology, and genetic engineering to indicate the success of a transfection or other procedure meant to introduce foreign DNA into a cell. Selectable markers are often antibiotic resistance genes; cells that have been subjected to a procedure to introduce foreign DNA are grown on a medium containing an antibiotic, and those cells that can grow have successfully taken up and expressed the introduced genetic material. Examples of selectable markers include: the Abicr gene or Neo gene from Tn5, which confers antibiotic resistance to geneticin.

A screenable marker may comprise a reporter gene, which allows the researcher to distinguish between wanted and unwanted cells. Certain embodiments of the present invention utilize reporter genes to indicate specific cell lineages. For example, the reporter gene can be located within expression elements and under the control of the ventricular- or atrial-selective regulatory elements normally associated with the coding region of a ventricular- or atrial-selective gene for simultaneous expression. A reporter allows the cells of a specific lineage to be isolated without placing them under drug or other selective pressures or otherwise risking cell viability.

Examples of such reporters include genes encoding cell surface proteins (e.g., CD4, HA epitope), fluorescent proteins, antigenic determinants and enzymes (e.g., β-galactosidase). The vector containing cells may be isolated, e.g., by FACS using fluorescently-tagged antibodies to the cell surface protein or substrates that can be converted to fluorescent products by a vector encoded enzyme.

In specific embodiments, the reporter gene is a fluorescent protein. A broad range of fluorescent protein genetic variants have been developed that feature fluorescence emission spectral profiles spanning almost the entire visible light spectrum. Mutagenesis efforts in the original Aequorea victoria jellyfish green fluorescent protein have resulted in new fluorescent probes that range in color from blue to yellow, and are some of the most widely used in vivo reporter molecules in biological research. Longer wavelength fluorescent proteins, emitting in the orange and red spectral regions, have been developed from the marine anemone, Discosoma striata, and reef corals belonging to the class Anthozoa. Still other species have been mined to produce similar proteins having cyan, green, yellow, orange, and deep red fluorescence emission. Developmental research efforts are ongoing to improve the brightness and stability of fluorescent proteins, thus improving their overall usefulness.

The cells in certain embodiments can be made to contain one or more genetic alterations by genetic engineering of the cells either before or after differentiation (US 2002/0168766). A cell is said to be “genetically altered”, “genetically modified” or “transgenic” when an exogenous nucleic acid or polynucleotide has been transferred into the cell by any suitable means of artificial manipulation, or where the cell is a progeny of the originally altered cell that has inherited the polynucleotide. For example, the cells can be processed to increase their replication potential by genetically altering the cells to express telomerase reverse transcriptase, either before or after they progress to restricted developmental lineage cells or terminally differentiated cells (U.S. Patent Application Publication 2003/0022367).

In certain embodiments, cells containing an exogenous nucleic acid construct may be identified in vitro or in vivo by including a marker in the expression vector, such as a selectable or screenable marker. Such markers would confer an identifiable change to the cell permitting easy identification of cells containing the expression vector, or help enrich or identify differentiated cardiac cells by using a tissue-specific promoter. For example, in the aspects of cardiomyocyte differentiation, cardiac-specific promoters may be used, such as promoters of cardiac troponin I (cTnI), cardiac troponin T (cTnT), sarcomeric myosin heavy chain (MHC), GATA-4, Nkx2.5, N-cadherin, □1-adrenoceptor, ANF, the MEF-2 family of transcription factors, creatine kinase MB (CK-MB), myoglobin, or atrial natriuretic factor (ANF). In aspects of neuron differentiation, neuron-specific promoters may be used, including but not limited to, TuJ-1, Map-2, Dcx or Synapsin. In aspects of hepatocyte differentiation, definitive endoderm- and/or hepatocyte-specific promoters may be used, including but not limited to, ATT, Cyp3a4, ASGPR, FoxA2, HNF4a or AFP.

Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to blasticidin, neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

In embodiments wherein cells are genetically modified, such as to add or reduce one or more features, the genetic modification may occur by any suitable method. For example, any genetic modification compositions or methods may be used to introduce exogenous nucleic acids into cells or to edit the genomic DNA, such as gene editing, homologous recombination or non-homologous recombination, RNA-mediated genetic delivery or any conventional nucleic acid delivery methods. Non-limiting examples of the genetic modification methods may include gene editing methods such as by CRISPR/CAS9, zinc finger nuclease, or TALEN technology.

Genetic modification may also include the introduction of a selectable or screenable marker that aid selection or screen or imaging in vitro or in vivo. Particularly, in vivo imaging agents or suicide genes may be expressed exogenously or added to starting cells or progeny cells. In further aspects, the methods may involve image-guided adoptive cell therapy.

### SPECIFIC EMBODIMENTS

In a specific embodiments of the disclosure there is provided a method of preparing a cell population comprising clonal invariant natural killer (iNKT) T cells comprising: a) selecting CD34+ cells from human peripheral blood cells (PBMCs); b) culturing the CD34+ cells with medium comprising growth factors that include c-kit ligand, flt-3 ligand, and human thrombopoietin (TPO) c) transducing the selected CD34+ cells with a lentiviral vector comprising a nucleic acid sequence encoding α-TCR, β-TCR, and thymidine kinase; d) introducing into the selected CD34+ cells Cas9 and gRNA for beta 2 microglobulin (B2M) and/or CTIIA to disrupt expression of B2M or CTIIA genes thus eliminating the surface expression of HLA-I and/or HLA-II molecules; e) culturing the transduced cells for 2-12 (or 2-10 or 6-12) weeks with an irradiated stromal cell line expressing an exogenous Notch ligand to expand iNKT cells in a 3D aggregate cell culture; f) selecting iNKT cells lacking surface expression of HLA-I/II molecules; and, g) culturing the selected iNKT cells with irradiated feeder cells. In particular embodiments, 10⁸-10¹³ iNKT cells are prepared from the selected CD34+ cells.

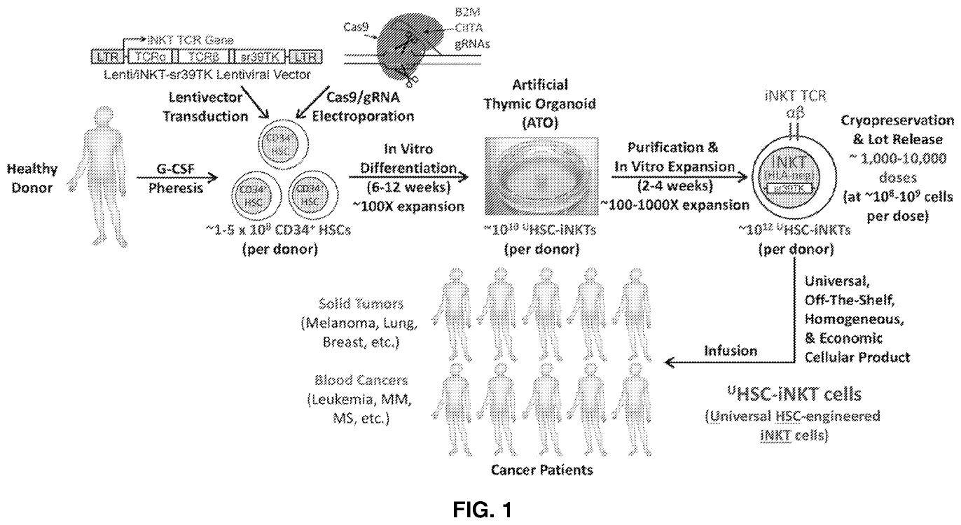

Thus, the disclosure encompasses an advanced HSC-based iNKT cell therapy that is universal and off-the-shelf (FIG. 1). Specifically, one can harvest G-CSF-mobilized CD34⁺ HSCs from healthy donors or from a cell repository. From a single donor, about 1-5×10⁸ HSCs can be collected. In specific cases, these HSCs are engineered in vitro with a Lenti/iNKT-sr39TK lentiviral vector and a CRISPR-Cas9/B2M-CIITA-gRNAs complex, then are differentiated into iNKT cells in an artificial thymic organoid (ATO) culture in 8 weeks. The iNKT cells may then be purified and further expanded in vitro for another 2-4 weeks, followed by cryopreservation and lot release. In specific aspects, about 10¹² iNKT cells are generated from HSCs of a single donor, which can be formulated into 1,000 to 10,000 doses (at ˜10⁸-10⁹ cells per dose, for example). The resulting cryopreserved cellular product, universal HSC-engineered iNKT (^{U}HSC-iNKT) cells, can then be readily stored and distributed to treat cancer patients off-the-shelf through allogenic adoptive cell transfer. Because iNKT cells can target multiple types of cancer without tumor antigen- and major histocompatibility complex (MHC)-restrictions, the ^{U}HSC-iNKT therapy is useful as a universal cancer therapy for treating multiple cancers and a large population of cancer patients, thus addressing the unmet medical need (FIG. 1) (Vivier et al., 2012; Berzins et al., 2011). Particularly, the disclosed HSC-iNKT therapy is useful to treat the many types of cancer that have been clinically implicated to be subject to iNKT cell regulation, including blood cancers (leukemia, multiple myeloma, and myelodysplastic syndromes), and solid tumors (melanoma, colon, lung, breast, and head and neck cancers) (Berzins et al., 2011).

The scientific embodiments underlying the ^{U}HSC-iNKT therapy are: 1) the lentiviral vector-mediated expression of a human iNKT T cell receptor (TCR) gene programs HSCs to differentiate into iNKT cells; 2) the inclusion of an sr39TK PET imaging/suicide gene allows for the monitoring of ^{U}HSC-iNKT cells in patients using PET imaging, as well as the depletion of these cells through ganciclovir (GCV) administration in case of a safety need; 3) the CRISPR-Cas9/B2M-CIITA-gRNAs-based gene editing of HSCs knocks out the B2M and CIITA genes, resulting in an HLA-I/II-negative cellular product suitable for allogenic infusion; 4) the ATO culture system supports the efficient development of human iNKT cells in vitro; 5) the manufacturing process is of high yield and high purity. The Examples section herein provides data supporting these scientific embodiments.

In specific cases, the manufacturing of ^{U}HSC-iNKT involves: 1) collection of G-CSF-mobilized leukopak; 2) purification of G-CSF-leukopak into CD34⁺ HSCs; 3) transduction of HSCs with lentiviral vector Lenti/iNKT-sr39TK; 4) gene editing of B2M and CIITA via CRISPR/Cas9; 5) in vitro differentiation into iNKT cells via ATO; 6) purification of iNKT cells; 7) in vitro cell expansion; 8) cell collection, formulation and cryopreservation. In a certain embodiment, there are two drug substances (Lenti/iNKT-sr39TK vector and ^{U}HSC-iNKT cells), and the final drug product may be the formulated and cryopreserved ^{U}HSC-iNKT in infusion bags, in specific cases.

Provided herein are examples of efficient protocols to generate ^{U}HSC-iNKT cells. Demonstrated herein is an efficient gene editing of HSCs to ablate the cell surface expression of class I HLA via knockout of B2M. Taking advantage of the multiplex editing CRISPR/Cas9, one can also simultaneously disrupt cell surface class II HLA expression via knockout of the gene for the class II transactivator (CIITA), a key regulator of HLA-II expression (Steimle et al., 1994), for example using a validated gRNA sequence (Abrahimi et al., 2015). Thus, incorporating this gene editing step to disrupt cell surface HLA-I and HLA-II expression and the microbeads purification step, we will generate ^{U}HSC-iNKT cells. Flow cytometric analysis may be used to measure the purity and the surface phenotypes of these engineered iNKT cells. The cell purity may be characterized by TCR In specific embodiments, this iNKT cell population is CD45RO⁺CD161⁺, indicative of memory and NK phenotypes, and contains both CD4¹⁻CD8⁻(CD4 single-positive), CD4⁻CD8⁺ (CD8 single-positive), and CD4⁻CD8⁻ (double-negative, DN) (Kronenberg and Gapin, 2002). CD62L expression may be analyzed, as a recent study indicated that its expression is associated with in vivo persistence of iNKT cells and their antitumor activity (Tian et al., 2016). One can compare these phenotypes of ^{U}HSC-iNKT with that iNKT from PBMCs. RNAseq may be employed to perform comparative gene expression analysis on ^{U}HSC-iNKT and PBMC iNKT cells.

IFN-γ production and cytotoxicity assays may be used to assess the functional properties of ^{U}HSC-iNKT, using PBMC iNKT as the benchmark control. ^{U}HSC-iNKT cells may be simulated with irradiated PBMCs that have been pulsed with αGC and supernatants harvested from one day stimulation may be subjected to IFN-γ ELISA (Smith et al., 2015). Intracellular cytokine staining (ICCS) of IFN-γ may be performed as well on iNKT cells after 6-hour stimulation. The cytotoxicity assay may be conducted by incubating effector ^{U}HSC-iNKT cells with aGC-loaded A375.CD1d target cells engineered to expression luciferase and GFP for 4 hours and cytotoxicity may be measured by a plate reader for its luminescence intensity. Because sr39TK is introduced as a PET/suicide gene, one canverify its function by incubating ^{U}HSC-iNKT with ganciclovir (GCV) and cell survival rate may be measured by a MTT assay and an Annexin V-based flow cytometric assay, for example.

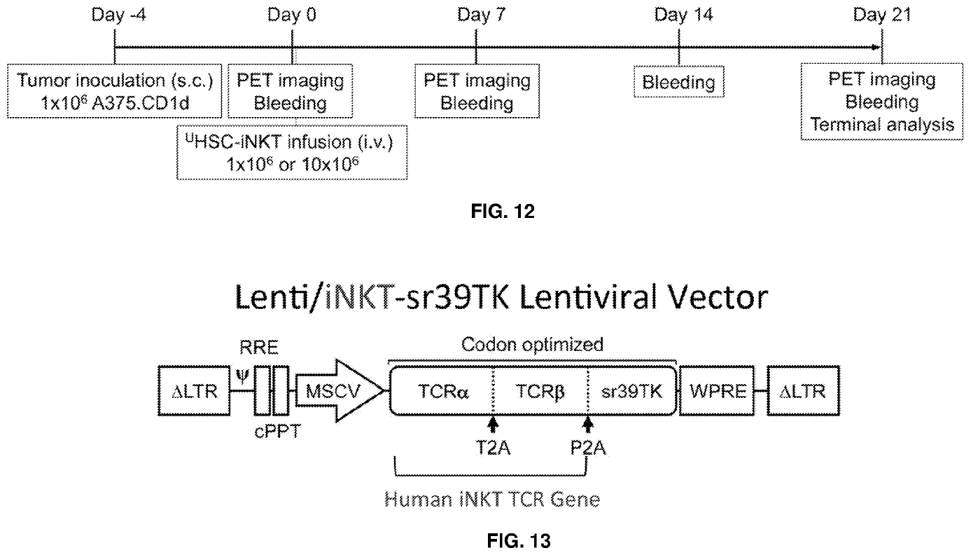

One can perform pharmacokinetics/Pharmacodynamics (PK/PD) studies. The PK/PD studies can determine in vivo in animal models the following: 1) expansion kinetics and persistence of infused ^{U}HSC-iNKT; 2) biodistribution of ^{U}HSC-iNKT in various tissues/organs; 3) ability of ^{U}HSC-iNKT to traffic to tumors and how this filtration relates to tumor growth. One can utilize immunodeficient NSG mice bearing A375.CD1d (A375.CD1d) tumors as the solid tumor animal model. A study design is outlined in FIG. 12. Two cell dose groups (1×10⁶ and 10×10⁶; n=8) may be investigated. The tumors may be inoculated (s.c.) on day −4 and the baseline PET imaging and bleeding may be conducted on day 0. Subsequently, ^{U}HSC-iNKT cells may be infused intravenously (i.v.) and monitored by 1) PET imaging in live animals on days 7 and 21; 2) periodic bleeding on days 7, 14 and 21; 3) end-point tissue collection after animal termination on day 21. Cell collected from various bleedings may be analyzed by flow cytometry; iNKT cells should be CD161⁺6B11⁺. One can examine the expression of other markers such as CD45RO, CD62L, and CD4 to see how iNKT subsets vary over the time. PET imaging via sr39TK will allow one to track the presence of iNKT cells in tumors and other tissues/organs such as bone, liver, spleen, thymus, etc. At the end of the study, tumors and mouse tissues including spleen, liver, brain, heart, kidney, lung, stomach, bone marrow, ovary, intestine, etc., may be harvested for qPCR analysis to examine the distribution of ^{U}HSC-iNKT cells.

One can characterize a mechanism of action (MOA) for the cells. iNKT cells are known to target tumor cells through either direct killing, or through the massive release of IFN-γ to direct NK and CD8 T cells to eradicate tumors (Fujii et al., 2013). An in vitro pharmacological study provides evidence of direct cytotoxicity. Here one can investigate the roles of NK and CD8 T cells in assisting antitumor reactivity in vivo. Tumor-bearing NSG mice (A375.CD1d or MM.1S.Luc) may be infused with either ^{U}HSC-iNKT alone (a dose chosen based on above in vivo study) or in combination with PBMCs (mismatched donor, 5×10⁶); owing to the MHC negativity of ^{U}HSC-iNKT, no allogenic immune response may occur between ^{U}HSC-iNKT and unrelated PBMCs. Tumor growth may be monitored and compared between with and without PBMC groups (n=8 per group). If a greater antitumor response is observed from the combination group, it may indicate that components in PBMCs, for example NK and/or CD8 T cells, play a role to boost therapeutic efficacy, in specific embodiments. To further determine their individual roles, PBMCs with depletion of NK (via CD56 beads), CD8 T cells (via CD8 beads), or myeloid (via CD14 beads) cells, may be co-infused along with ^{U}HSC-iNKT cells into tumor-bearing mice. Immune checkpoint inhibitors such as PD-1 and CTLA-4 have been suggested to regulate iNKT cell function (Pilones et al., 2012; Durgan et al., 2011). Through adding anti-PD-1 or anti-CTLA-4 treatment to the ^{U}HSC-iNKT therapy, one can determine how these molecules modulate ^{U}HSC-iNKT therapy and provide information on the design of combination cancer therapy.

Particular vectors may be utilized for the production of ^{U}HSC-iNKT cells and/or their use. One can utilize a vector for genetic engineering of HSCs into iNKT cells such as an HIV-1 derived lentiviral vector Lenti/iNKT-sr39TK encoding a human iNKT TCR gene along with an sr39TK PET imaging/suicide gene (FIG. 13). Components of this third generation self-inactivating (SIN) vector are: 1) 3′ self-inactivating long-term repeats (ΔLTR); 2) Ψ region vector genome packaging signal; 3) Rev Responsive Element (RRE) to enhance nuclear export of unspliced vector RNA; 4) central PolyPurine Tract (cPPT) to facilitate unclear import of vector genomes; 5) expression cassette of the a chain gene (TCRα) and β chain gene (TCRβ) of a human iNKT TCR, as well as the PET/suicide gene sr39TK (Gscheng et al., 2014) driven by internal promoter from the murine stem cell virus (MSCV). The iNKT TCRα and TCRβ and sr39TK genes are all codon-optimized and linked by 2A self-cleaving sequences (T2A and P2A) to achieve their optimal co-expression (Gscheng et al., 2014).

Regarding quality control of the vector, a series of QC assays may be performed to ensure that the vector product is of high quality. Those standard assays such as vector identity, vector physical titer, and vector purity (sterility, mycoplasma, viral contaminants, replication-competent lentivirus (RCL) testing, endotoxin, residual DNA and benzonase) may be conducted at IU VPF and provided in the Certificate of Analysis (COA). Additional QC assays that may be performed include 1) the transduction/biological titer (by transducing HT29 cells with serial dilutions and performing ddPCR, ≥1×10⁶ TU/ml); 2) the vector provirus integrity (by sequencing the vector-integrated portion of genomic DNA of transduced HT29 cells, same to original vector plasmid sequence); 3) the vector function. The vector function may be measured by transducing human PBMC T cells (Chodon et al., 2014). The expression of iNKT TCR gene may be detected by staining with the 6B11 specific for iNKT TCR (Montoya et al., 2007). The functionality of expressed iNKT TCRs will be analyzed by IFN-γ production in response to αGalCer stimulation (Watarai et al., 2008). The expression and functionality of sr39TK gene may be analyzed by penciclovir update assay and GCV killing assay (Gschweng et al., 2014. The stability of the vector stock (stored in −80 freezer) may be tested every 3 months by measuring its transduction titer.

### VI. Specific Cell Manufacturing and Product Formulation

An overview and a specific manufacturing process for ^{U}HSC-iNKT cells is provided. In specific embodiments, ^{U}HSC-iNKT cells are the key drug substance that functions as “living drug” to target and fight disease in a mammal, including fight tumor cells, for example. In particular embodiments, they are generated by in vitro differentiation and expansion of genetically modified donor HSCs. Data demonstrates a novel and efficient protocol to produce the cells in a laboratory scale, and in specific embodiments the cells are made as an “off-the-shelf” cell product in a GMP-comparable manufacturing process. In specific cases, production scale is 10¹² cells per batch, which is estimated to treat 1000-10,000 patients.

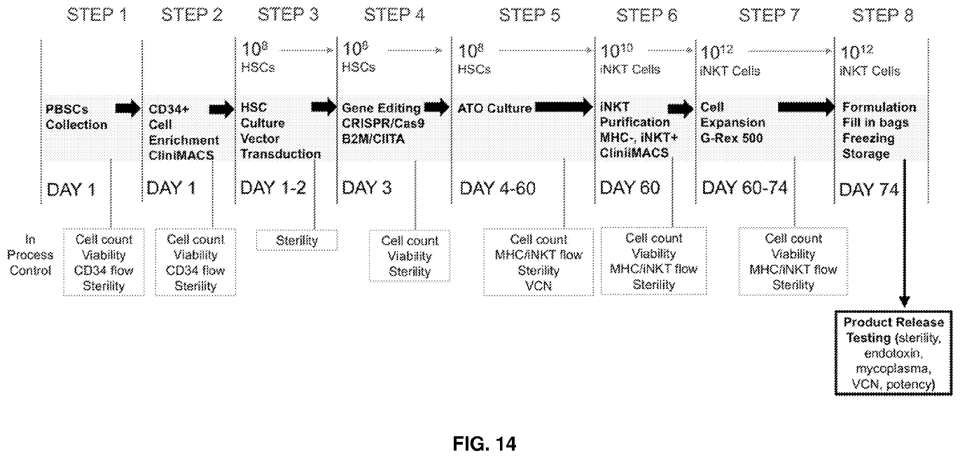

An example of a cell manufacturing process is provided. One cell manufacturing process is outlined in FIG. 14, with examples of timelines and “In-Process-Control (IPC)” measurements for each process step, in at least some cases. Step 1 is to harvest donor G-CSF-mobilized PBSCs in blood collection facilities, which has become a routine procedure in many hospitals (Deotare et al., 2015). One can obtain fresh PBSCs in Leukopaks from the HemaCare for this project; HemaCare has IRB-approved collection protocols and donor consents and can support clinical trials and commercial product manufacturing. Step 2 is to enrich CD34⁺ HSCs from PBSCs using a CliniMACS system; one can use such a system located at the UCLA GMP facility to complete this step and one can yield at least 10⁸ CD34⁺ cells, in specific aspeces. CD34⁻ cells may be collected and stored as well (they may be used as PBMC feeder in Step 7).

Step 3 involves the HSC culture and vector transduction. CD34⁺ cells may be cultured in X-VIVO15 medium supplemented with 1% HAS (USP) and growth factor cocktails (c-kit ligand, flt-3 ligand and tpo; 50 ng/ml each) for 12 hrs in flasks coated with retronectin, followed by addition of the Lenti/iNKT-sr39TK vector for additional 8 hrs (Gschweng et al., 2014). Vector integration copies (VCN) may be measured by sampling ˜50 colonies formed in the methylcellulose assay for transduced cells and the average vector copy number per cell may be determined using ddPCR (Nolta et al., 1994). In specific cases the procedure is optimized and >50% transduction is routinely achieved with VCN=1-3 per cell.

Step 4 is to utilize the powerful CRISPR/Cas9 multiplex gene editing method to target the genomic loci of both B2M and CIITA in HSCs and disrupt their gene expression (Ren et al., 2017; Liu et al., 2017), and iNKT cells derived from edited HSCs will lack the MHC/HLA expression, thereby avoiding the rejection by the host immune system. Initial data has demonstrated the success of the B2M disruption for CD34⁺ HSCs with high efficiency (˜75% by flow analysis) via electroporation of Cas9/B2M-gRNA. B2M/CIITA double knockout may be achieved by electroporation of a mixture of RNPs (Cas9/B2M-gRNA and Cas9/CIITA-gRNA_(Abrahimi et al., 2015)). One can optimize and validate this process (Gundry et al., 2016) by varying electroporation parameters, ratios of two RNPs, stem cell culture time (24, 48, or 72 hrs post-transduction) prior to electroporation, etc; one can use the high fidelity Cas9 protein (Slaymaker et al., 2016; Tsai and Joung, 2016) from IDT to minimize the “off-target” effect. Exemplary evaluation parameters may be viability, deletion (indel) frequency (on-target efficiency) measured by a T7E1 assay and next-generation sequencing (NGS) targeting the B2M and CIITA sites, MHC expression by flow cytometry, and hematopoietic function of edited HSCs measured by the colony formation unit (CFU) assay.

Step 5 is to in vitro differentiate modified CD34⁺ HSCs into iNKT cells via the artificial thymic organoid (ATO) culture¹. Initial studies have shown that functional iNKT cells can be efficiently generated from HSCs engineered to express iNKT TCRs. Building upon this data, one can test and validate an 8-week, GMP-compatible ATO culture process to produce 10¹⁰ iNKT cells from 10⁸ modified CD34⁺ HSCs. ATO involves pipetting a cell slurry (5 μl) containing mixture of HSCs (5×10⁴) and irradiated (80 Gy) MS5-hDLL1 stromal cells (10⁶) as a drop format onto a 0.4-μm Millicell transwell insert, followed by placing the insert into a 6-well plate containing 1 ml RB27 medium¹; medium may be changed every 4 days for 8 weeks. Considering 3 ATOs per insert, approximately 170 six-well plates for each batch production may be utilized. One can use an automated programmable pipetting/dispensing system (epMontion 5070f from Eppendorf) placed in biosafety cabinet for plating ATO droplets and medium exchange; a 2-hr operation may be needed for completing 170 plates each round. At the end of ATO culture, iNKT cells may be harvested and characterized. In specific embodiments a component of ATO is the MS5-hDLL1 stromal cell line that is constructed by lentiviral transduction to express human DLL1 followed by cell sorting. In preparation for certain GMP processes, one can perform a single cell clonal selection process on this polyclonal cell population to establish several clonal MS5-hDLL1 cell lines, from which one can choose an efficient one (evaluated by ATO culture) and use it to generate a master cell bank. Such a bank may be used to supply irradiated stromal cells for future clinical grade ATO culture.

Step 6 is to purify ATO-derived iNKT cells using the CliniMACS system. This step purification is to deplete MHCI⁺ and MHCII⁺ cells and enrich iNKT⁺ cells. Anti-MHCI and anti-MHCII beads may be prepared by incubating Miltenyi anti-Biotin beads with commercially available biotinylated anti-MHCI (clone W6/32, HLA-A, B, C), anti-B2M (clone 2M2), and anti-MHCII (clone Tu39, HLA-DR, DP, DQ), and anti-TCR Vα24-Jα18 (clone 6B11). 6B11 directly-coated microbeads are also available from Miltenyi; anti-iNKT beads are available from Miltenyi Biotec. Harvested iNKT cells may be labeled by anti-MHC bead mixtures and washed twice and MHCI⁺ and/or MHCII⁺ cells may be depleted using the CliniMACS depletion program; if necessary, this depletion step can be repeated to further remove residual MHC⁺ cells. Subsequently, iNKT cells may be further purified using the standard anti-iNKT beads and the CliniMACS enrichment program. The cell purity may be measured by flow cytometry, for example.

Step 7 is to expand purified iNKT cells in vitro. Starting from 10¹⁰ cells, one can expand into 10¹² iNKT cells using an already validated PBMC feeder-based in vitro expansion protocol (Yamasaki et al., 2011; Heczey et al., 2014). One can evaluate a G-Rex-based bioprocess for this cell expansion. G-Rex is a cell growth flask with a gas-permeable membrane at the bottom allowing more efficient gas exchange; A G-Rex500M flask has the capacity to support a 100-fold cell expansion in 10 days (Vera et al., 2010; Bajgain et al., 2014; Jin et al., 2012). The stored CD34⁻ cells (used as feeder cells) from the Step 1 may be thawed, pulsed with αGalCer (100 ng/ml), and irradiated (40 Gy). iNKT cells may be mixed with irradiated feeder cells (1:4 ratio), seeded into G-Rex flasks (1.25×10⁸ iNKT each, 80 flasks), and allowed to expand for 2 weeks. IL-2 (200 U/ml) will be added every 2-3 days and one medium exchange will occur at day 7; all medium manipulation may be achieved by peristaltic pumps. This expansion process is GMP-compatible because a similar PBMC feeder-based expansion procedure (termed rapid expansion protocol) has been already utilized to produce therapeutic T cells for many clinical trials (Dudley et al., 2008; Rosenberg et al., 2008).

Step 8 is to formulate the harvested iNKT cells from Step 7 (the active drug component) into cell suspension for direct infusion. After at least 3 rounds of extensive washing, cells from Step 7 may be counted and suspended into an infusion/cold storage-compatible solution (10⁷-10⁸ cells/ml), which is composed of Plasma-Lyte A Injection (31.25% v/v), Dextrose and Sodium Chloride Injection (31.25% v/v), Human Albumin (20% v/v), Dextran 40 in Dextrose Inject (10%, v/v) and Cryosery DMSO (7.5%, v/v); this solution has been used to formulate tisagenlecleucel, an approved T cell product from Novartis (Grupp et al., 2013). Once filled into FDA-approved freezing bags (such as CryoMACS freezing bags from Miltenyi Biotec), the product may be frozen in a controlled rate freezer and stored in a liquid nitrogen freezer. One can perform validation and/or optimization studies by measuring viability and recovery to ensure that this formulation is appropriate for an ^{U}HSC-iNKT cell product.

Various IPC assays such as cell counting, viability, sterility, mycoplasma, identity, purity, VCN, etc.) may be incorporated into the proposed bioprocess to ensure a high-quality production. Testing may include the following: 1) appearance (color, opacity); 2) cell viability and count; 3) identity and VCN by qPCR for iNKT TCR; 4) purity by iNKT positivity and B2M negativity; 5) endotoxins; 6) sterility; 7) mycoplasma; 8) potency measured by IFN-γ release in response to αGalCer stimulation; 9) RCL (replication-competent lentivirus) (Cornetta et al, 2011). Most of these assays are either standard biological assays or specific assays unique to this product. Product stability testing may be performed by periodically thawing LN-stored bags and measuring their cell viability, purity, recovery, potency (IFN-γ release) and sterility. In particular embodiments, the product is stable for at least one year.

### A. Source of Starting Cells

Starting cells such as pluripotent stem cells or hematopoietic stem or progenitor cells may be used in certain compositions or methods for differentiation along a selected T cell lineage. Stromal cells may be used to co-culture with the stem or progenitor cells.

### B. Stromal Cells

Stromal cells are connective tissue cells of any organ, for example in the bone marrow, thymus, uterine mucosa (endometrium), prostate, and the ovary. They are cells that support the function of the parenchymal cells of that organ. Fibroblasts (also known as mesenchymal stromal cells/MSC) and pericytes are among the most common types of stromal cells.

The interaction between stromal cells and tumor cells is known to play a major role in cancer growth and progression. In addition, by regulating locally cytokine networks (e.g. M-CSF, LIF), bone marrow stromal cells have been described to be involved in human haematopoiesis and inflammatory processes.

Stromal cells in the bone marrow, thymus, and other hematopoietic organs regulate hematopoietic and immune cell development though cell-cell ligand-receptor interactions and through the release of soluble factors including cytokines and chemokines. Stromal cells in these tissues form niches that regulate stem cell maintenance, lineage specification and commitment, and differentiation to effector cell types.

Stroma is made up of the non-malignant host cells. Stromal cells also provides an extracellular matrix on which tissue-specific cell types, and in some cases tumors, can grow.

### C. Hematopoietic Stem and Progenitor Cells

Due to the significant medical potential of hematopoietic stem and progenitor cells, substantial work has been done to try to improve methods for the differentiation of hematopoietic progenitor cells from embryonic stem cells. In the human adult, hematopoietic stem cells present primarily in bone marrow produce heterogeneous populations of hematopoietic (CD34+) progenitor cells that differentiate into all the cells of the blood system. In an adult human, hematopoietic progenitors proliferate and differentiate resulting in the generation of hundreds of billions of mature blood cells daily. Hematopoietic progenitor cells are also present in cord blood. In vitro, human embryonic stem cells may be differentiated into hematopoietic progenitor cells. Hematopoietic progenitor cells may also be expanded or enriched from a sample of peripheral blood as described below. The hematopoietic cells can be of human origin, murine origin or any other mammalian species.

Isolation of hematopoietic progenitor cells include any selection methods, including cell sorters, magnetic separation using antibody-coated magnetic beads, packed columns; affinity chromatography; cytotoxic agents joined to a monoclonal antibody or used in conjunction with a monoclonal antibody, including but not limited to, complement and cytotoxins; and “panning” with antibody attached to a solid matrix, e.g., plate, or any other convenient technique.

The use of separation or isolation techniques include, but are not limited to, those based on differences in physical (density gradient centrifugation and counter-flow centrifugal elutriation), cell surface (lectin and antibody affinity), and vital staining properties (mitochondria-binding dye rho123 and DNA-binding dye Hoechst 33342). Techniques providing accurate separation include but are not limited to, FACS (Fluorescence-activated cell sorting) or MACS (Magnetic-activated cell sorting), which can have varying degrees of sophistication, e.g., a plurality of color channels, low angle and obtuse light scattering detecting channels, impedance channels, etc.

The antibodies utilized in the preceding techniques or techniques used to assess cell type purity (such as flow cytometry) can be conjugated to identifiable agents including, but not limited to, enzymes, magnetic beads, colloidal magnetic beads, haptens, fluorochromes, metal compounds, radioactive compounds, drugs or haptens. The enzymes that can be conjugated to the antibodies include, but are not limited to, alkaline phosphatase, peroxidase, urease and β-galactosidase. The fluorochromes that can be conjugated to the antibodies include, but are not limited to, fluorescein isothiocyanate, tetramethylrhodamine isothiocyanate, phycoerythrin, allophycocyanins and Texas Red. For additional fluorochromes that can be conjugated to antibodies, see Haugland, Molecular Probes: Handbook of Fluorescent Probes and Research Chemicals (1992-1994). The metal compounds that can be conjugated to the antibodies include, but are not limited to, ferritin, colloidal gold, and particularly, colloidal superparamagnetic beads. The haptens that can be conjugated to the antibodies include, but are not limited to, biotin, digoxygenin, oxazalone, and nitrophenol. The radioactive compounds that can be conjugated or incorporated into the antibodies are known to the art, and include but are not limited to technetium 99m (99TC), 125I and amino acids comprising any radionuclides, including, but not limited to, 14C, 3H and 35S.

Other techniques for positive selection may be employed, which permit accurate separation, such as affinity columns, and the like. The method should permit the removal to a residual amount of less than about 20%, preferably less than about 5%, of the non-target cell populations.

Cells may be selected based on light-scatter properties as well as their expression of various cell surface antigens. The purified stem cells have low side scatter and low to medium forward scatter profiles by FACS analysis. Cytospin preparations show the enriched stem cells to have a size between mature lymphoid cells and mature granulocytes.

It also is possible to enrich the inoculation population for CD34+ cells prior to culture, using for example, the method of Sutherland et al. (1992) and that described in U.S. Pat. No. 4,714,680. For example, the cells are subject to negative selection to remove those cells that express lineage specific markers. In an illustrative embodiment, a cell population may be subjected to negative selection for depletion of non-CD34+ hematopoietic cells and/or particular hematopoietic cell subsets. Negative selection can be performed on the basis of cell surface expression of a variety of molecules, including T cell markers such as CD2, CD4 and CD8; B cell markers such as CD10, CD19 and CD20; monocyte marker CD14; the NK cell marker CD2, CD16, and CD56 or any lineage specific markers. Negative selection can be performed on the basis of cell surface expression of a variety of molecules, such as a cocktail of antibodies (e.g., CD2, CD3, CD11b, CD14, CD15, CD16, CD19, CD56, CD123, and CD235a) which may be used for separation of other cell types, e.g., via MACS or column separation.

As used herein, lineage-negative (LIN−) refers to cells lacking at least one marker associated with lineage committed cells, e.g., markers associated with T cells (such as CD2, 3, 4 and 8), B cells (such as CD10, 19 and 20), myeloid cells (such as CD14, 15, 16 and 33), natural killer (“NK”) cells (such as CD2, 16 and 56), RBC (such as glycophorin A), megakaryocytes (CD41), mast cells, eosinophils or basophils or other markers such as CD38, CD71, and HLA-DR. Preferably the lineage specific markers include, but are not limited to, at least one of CD2, CD14, CD15, CD16, CD19, CD20, CD33, CD38, HLA-DR and CD71. More preferably, LIN− will include at least CD14 and CD15. Further purification can be achieved by positive selection for, e.g., c-kit+ or Thy-1+. Further enrichment can be obtained by use of the mitochondrial binding dye rhodamine 123 and selection for rhodamine+ cells, by methods known in the art. A highly enriched composition can be obtained by selective isolation of cells that are CD34+, preferably CD34+LIN−, and most preferably, CD34+ Thy-1+ LIN−. Populations highly enriched in stem cells and methods for obtaining them are well known to those of skill in the art, see e.g., methods described in PCT Patent Application Nos. PCT/US94/09760; PCT/US94/08574 and PCT/US94/10501.

Various techniques may be employed to separate the cells by initially removing cells of dedicated lineage. Monoclonal antibodies are particularly useful for identifying markers associated with particular cell lineages and/or stages of differentiation. The antibodies may be attached to a solid support to allow for crude separation. The separation techniques employed should maximize the retention of viability of the fraction to be collected. Various techniques of different efficacy may be employed to obtain “relatively crude” separations. Such separations are where up to 10%, usually not more than about 5%, preferably not more than about 1%, of the total cells present are undesired cells that remain with the cell population to be retained. The particular technique employed will depend upon efficiency of separation, associated cytotoxicity, ease and speed of performance, and necessity for sophisticated equipment and/or technical skill.

Selection of the progenitor cells need not be achieved solely with a marker specific for the cells. By using a combination of negative selection and positive selection, enriched cell populations can be obtained.

### D. Sources of Blood Cells

Hematopoietic stem cells (HSCs) normally reside in the bone marrow but can be forced into the blood, a process termed mobilization used clinically to harvest large numbers of HSCs in peripheral blood. One example of a mobilizing agent of choice is granulocyte colony-stimulating factor (G-CSF).

CD34+ hematopoietic stem cells or progenitors that circulate in the peripheral blood can be collected by apheresis techniques either in the unperturbed state, or after mobilization following the external administration of hematopoietic growth factors like G-CSF. The number of the stem or progenitor cells collected following mobilization is greater than that obtained after apheresis in the unperturbed state. In a particular aspect of the present invention, the source of the cell population is a subject whose cells have not been mobilized by extrinsically applied factors because there is no need to enrich hematopoietic stem cells or progenitor cells in vivo.

Populations of cells for use in the methods described herein may be mammalian cells, such as human cells, non-human primate cells, rodent cells (e.g., mouse or rat), bovine cells, ovine cells, porcine cells, equine cells, sheep cell, canine cells, and feline cells or a mixture thereof. Non-human primate cells include rhesus macaque cells. The cells may be obtained from an animal, e.g., a human patient, or they may be from cell lines. If the cells are obtained from an animal, they may be used as such, e.g., as unseparated cells (i.e., a mixed population); they may have been established in culture first, e.g., by transformation; or they may have been subjected to preliminary purification methods. For example, a cell population may be manipulated by positive or negative selection based on expression of cell surface markers; stimulated with one or more antigens in vitro or in vivo; treated with one or more biological modifiers in vitro or in vivo; or a combination of any or all of these.

Populations of cells include peripheral blood mononuclear cells (PBMC), whole blood or fractions thereof containing mixed populations, spleen cells, bone marrow cells, tumor infiltrating lymphocytes, cells obtained by leukapheresis, biopsy tissue, lymph nodes, e.g., lymph nodes draining from a tumor. Suitable donors include immunized donors, non-immunized (naive) donors, treated or untreated donors. A “treated” donor is one that has been exposed to one or more biological modifiers. An “untreated” donor has not been exposed to one or more biological modifiers.

For example, peripheral blood mononuclear cells (PBMC) can be obtained as described according to methods known in the art. Examples of such methods are discussed by Kim et al. (1992); Biswas et al. (1990); Biswas et al. (1991).

Methods of obtaining precursor cells from populations of cells are also well known in the art. Precursor cells may be expanded using various cytokines, such as hSCF, hFLT3, and/or IL-3 (Akkina et al., 1996), or CD34+ cells may be enriched using MACS or FACS. As mentioned above, negative selection techniques may also be used to enrich CD34+ cells.

It is also possible to obtain a cell sample from a subject, and then to enrich it for a desired cell type. For example, PBMCs and/or CD34+ hematopoietic cells can be isolated from blood as described herein. Cells can also be isolated from other cells using a variety of techniques, such as isolation and/or activation with an antibody binding to an epitope on the cell surface of the desired cell type. Another method that can be used includes negative selection using antibodies to cell surface markers to selectively enrich for a specific cell type without activating the cell by receptor engagement.

Bone marrow cells may be obtained from iliac crest, femora, tibiae, spine, rib or other medullary spaces. Bone marrow may be taken out of the patient and isolated through various separations and washing procedures. An exemplary procedure for isolation of bone marrow cells comprises the following steps: a) centrifugal separation of bone marrow suspension in three fractions and collecting the intermediate fraction, or buffycoat; b) the buffycoat fraction from step (a) is centrifuged one more time in a separation fluid, commonly Ficoll (a trademark of Pharmacia Fine Chemicals AB), and an intermediate fraction which contains the bone marrow cells is collected; and c) washing of the collected fraction from step (b) for recovery of re-transfusable bone marrow cells.

### E. Pluripotent Stem Cells

The cells suitable for the compositions and methods described herein may be hematopoietic stem and progenitor cells may also be prepared from differentiation of pluripotent stem cells in vitro. In some embodiments, the cells used in the methods described herein are pluripotent stem cells (embryonic stem cells or induced pluripotent stem cells) directly seeded into the ATOs. In further embodiments, the cells used in the methods and compositions described herein are a derivative or progeny of the PSC such as, but not limited to mesoderm progenitors, hemato-endothelial progenitors, or hematopoietic progenitors.

The term “pluripotent stem cell” refers to a cell capable of giving rise to cells of all three germinal layers, that is, endoderm, mesoderm and ectoderm. Although in theory a pluripotent stem cell can differentiate into any cell of the body, the experimental determination of pluripotency is typically based on differentiation of a pluripotent cell into several cell types of each germinal layer. In some embodiments, a pluripotent stem cell is an embryonic stem (ES) cell derived from the inner cell mass of a blastocyst. In other embodiments, the pluripotent stem cell is an induced pluripotent stem cell derived by reprogramming somatic cells. In certain embodiments, the pluripotent stem cell is an embryonic stem cell derived by somatic cell nuclear transfer.

Embryonic stem (ES) cells are pluripotent cells derived from the inner cell mass of a blastocyst. ES cells can be isolated by removing the outer trophectoderm layer of a developing embryo, then culturing the inner mass cells on a feeder layer of non-growing cells. Under appropriate conditions, colonies of proliferating, undifferentiated ES cells are produced. The colonies can be removed, dissociated into individual cells, then replated on a fresh feeder layer. The replated cells can continue to proliferate, producing new colonies of undifferentiated ES cells. The new colonies can then be removed, dissociated, replated again and allowed to grow. This process of “subculturing” or “passaging” undifferentiated ES cells can be repeated a number of times to produce cell lines containing undifferentiated ES cells (U.S. Pat. Nos. 5,843,780; 6,200,806; 7,029,913). A “primary cell culture” is a culture of cells directly obtained from a tissue such as the inner cell mass of a blastocyst. A “subculture” is any culture derived from the primary cell culture.

Methods for obtaining mouse ES cells are well known. In one method, a preimplantation blastocyst from the 129 strain of mice is treated with mouse antiserum to remove the trophoectoderm, and the inner cell mass is cultured on a feeder cell layer of chemically inactivated mouse embryonic fibroblasts in medium containing fetal calf serum. Colonies of undifferentiated ES cells that develop are subcultured on mouse embryonic fibroblast feeder layers in the presence of fetal calf serum to produce populations of ES cells. In some methods, mouse ES cells can be grown in the absence of a feeder layer by adding the cytokine leukemia inhibitory factor (LIF) to serum-containing culture medium (Smith, 2000). In other methods, mouse ES cells can be grown in serum-free medium in the presence of bone morphogenetic protein and LIF (Ying et al., 2003).

Human ES cells can be obtained from blastocysts using previously described methods (Thomson et al., 1995; Thomson et al., 1998; Thomson and Marshall, 1998; Reubinoff et al, 2000.) In one method, day-5 human blastocysts are exposed to rabbit anti-human spleen cell antiserum, then exposed to a 1:5 dilution of Guinea pig complement to lyse trophectoderm cells. After removing the lysed trophectoderm cells from the intact inner cell mass, the inner cell mass is cultured on a feeder layer of gamma-inactivated mouse embryonic fibroblasts and in the presence of fetal bovine serum. After 9 to 15 days, clumps of cells derived from the inner cell mass can be chemically (i.e. exposed to trypsin) or mechanically dissociated and replated in fresh medium containing fetal bovine serum and a feeder layer of mouse embryonic fibroblasts. Upon further proliferation, colonies having undifferentiated morphology are selected by micropipette, mechanically dissociated into clumps, and replated (see U.S. Pat. No. 6,833,269). ES-like morphology is characterized as compact colonies with apparently high nucleus to cytoplasm ratio and prominent nucleoli. Resulting ES cells can be routinely passaged by brief trypsinization or by selection of individual colonies by micropipette. In some methods, human ES cells can be grown without serum by culturing the ES cells on a feeder layer of fibroblasts in the presence of basic fibroblast growth factor (Amit et al., 2000). In other methods, human ES cells can be grown without a feeder cell layer by culturing the cells on a protein matrix such as Matrigel™ or laminin in the presence of “conditioned” medium containing basic fibroblast growth factor (Xu et al., 2001). The medium is previously conditioned by coculturing with fibroblasts.

Methods for the isolation of rhesus monkey and common marmoset ES cells are also known (Thomson, and Marshall, 1998; Thomson et al., 1995; Thomson and Odorico, 2000).

Another source of ES cells are established ES cell lines. Various mouse cell lines and human ES cell lines are known and conditions for their growth and propagation have been defined. For example, the mouse CGR8 cell line was established from the inner cell mass of mouse strain 129 embryos, and cultures of CGR8 cells can be grown in the presence of LIF without feeder layers. As a further example, human ES cell lines H1, H7, H9, H13 and H14 were established by Thompson et al. In addition, subclones H9.1 and H9.2 of the H9 line have been developed.

The source of ES cells can be a blastocyst, cells derived from culturing the inner cell mass of a blastocyst, or cells obtained from cultures of established cell lines. Thus, as used herein, the term “ES cells” can refer to inner cell mass cells of a blastocyst, ES cells obtained from cultures of inner mass cells, and ES cells obtained from cultures of ES cell lines.

Induced pluripotent stem (iPS) cells are cells which have the characteristics of ES cells but are obtained by the reprogramming of differentiated somatic cells. Induced pluripotent stem cells have been obtained by various methods. In one method, adult human dermal fibroblasts are transfected with transcription factors Oct4, Sox2, c-Myc and Klf4 using retroviral transduction (Takahashi et al., 2007). The transfected cells are plated on SNL feeder cells (a mouse cell fibroblast cell line that produces LIF) in medium supplemented with basic fibroblast growth factor (bFGF). After approximately 25 days, colonies resembling human ES cell colonies appear in culture. The ES cell-like colonies are picked and expanded on feeder cells in the presence of bFGF.

Based on cell characteristics, cells of the ES cell-like colonies are induced pluripotent stem cells. The induced pluripotent stem cells are morphologically similar to human ES cells, and express various human ES cell markers. Also, when growing under conditions that are known to result in differentiation of human ES cells, the induced pluripotent stem cells differentiate accordingly. For example, the induced pluripotent stem cells can differentiate into cells having neuronal structures and neuronal markers.

In another method, human fetal or newborn fibroblasts are transfected with four genes, Oct4, Sox2, Nanog and Lin28 using lentivirus transduction (Yu et al., 2007). At 12-20 days post infection, colonies with human ES cell morphology become visible. The colonies are picked and expanded. The induced pluripotent stem cells making up the colonies are morphologically similar to human ES cells, express various human ES cell markers, and form teratomas having neural tissue, cartilage and gut epithelium after injection into mice.

Methods of preparing induced pluripotent stem cells from mouse are also known (Takahashi and Yamanaka, 2006). Induction of iPS cells typically require the expression of or exposure to at least one member from Sox family and at least one member from Oct family. Sox and Oct are thought to be central to the transcriptional regulatory hierarchy that specifies ES cell identity. For example, Sox may be Sox-1, Sox-2, Sox-3, Sox-15, or Sox-18; Oct may be Oct-4. Additional factors may increase the reprogramming efficiency, like Nanog, Lin28, Klf4, or c-Myc; specific sets of reprogramming factors may be a set comprising Sox-2, Oct-4, Nanog and, optionally, Lin-28; or comprising Sox-2, Oct4, Klf and, optionally, c-Myc.

IPS cells, like ES cells, have characteristic antigens that can be identified or confirmed by immunohistochemistry or flow cytometry, using antibodies for SSEA-1, SSEA-3 and SSEA-4 (Developmental Studies Hybridoma Bank, National Institute of Child Health and Human Development, Bethesda Md.), and TRA-1-60 and TRA-1-81 (Andrews et al., 1987). Pluripotency of embryonic stem cells can be confirmed by injecting approximately 0.5-10×10⁶ cells into the rear leg muscles of 8-12 week old male SCID mice. Teratomas develop that demonstrate at least one cell type of each of the three germ layers.

### VII. Methods of Using the Cells

The ^{U}HSC-iNKT cells of the disclosure may or may not be utilized directly after production. In some cases they are stored for later purpose. In any event, they may be utilized in therapeutic or preventative applications for a mammalian subject (human, dog, cat, horse, etc.) such as a patient. The patient may be in need of cell therapy for a medical condition of any kind, including allogeneic cell therapy.

Methods of treating a patient with a therapeutically effective amount of ^{U}HSC-iNKT cells of the disclosure comprise administering the cells or clonal populations thereof to the patient The cells or cell populations may be allogeneic with respect to the patient. The patient does not exhibit signs of depletion of the cells or cell population, in particular embodiments. The patient may or may not have cancer and/or a disease or condition involving inflammation.

In specific embodiments wherein the patient has cancer, tumor cells of the cancer patient are killed after administering the cells or cell population to the patient. In specific cases wherein the patient has inflammation, the inflammation is reduced following administering the cells or cell population to the patient. In specific embodiments of the methods of treatment, the method further comprises administering to the patient a compound that initiates the suicide gene product.

For patients with cancer, once infused into patients it is expected that this cell product can employ multiple mechanisms to target and eradicate tumor cells. The infused cells can directly recognize and kill CD1d⁺ tumor cells through cytotoxicity. They can secrete cytokines such as IFN-γ to activate NK cells to kill HLA-negative tumor cells, and also activate DCs which then stimulate cytotoxic T cells to kill HLA-positive tumor cells. Accordingly, we plan a series of in vitro and in vivo studies to demonstrate the pharmacological efficacy of this cell product for cancer therapy.

Because the ^{U}HSC-iNKT cells can target a large range of cancers without tumor antigen- and MHC-restrictions, an off-the-shelf ^{U}HSC-iNKT cellular product is useful as a general cancer immunotherapy for treating any type of cancer and a large population of cancer patients. In specific cases, the present therapy is useful for patients with cancers that have been clinically indicated to be subject to iNKT cell regulation, including multiple types of solid tumors (melanoma, colon, lung, breast, and head and neck cancers) and blood cancers (leukemia, multiple myeloma, and myelodysplastic syndromes), for example.

In some embodiments of any of the above-disclosed methods, the subject has or is at risk of having an autoimmune disease, graft versus host disease (GVHD), or graft rejection. The subject may be one diagnosed with such disease or one that has been determined to have a pre-disposition to such disease based on genetic or family history analysis. The subject may also be one that is preparing to or has undergone a transplant. In some embodiments, the method is for treating an autoimmune disease, GVHD, or graft rejection.

Individuals treated with the present cell therapy may or may not have been treated for the particular medical condition prior to receiving the ^{U}HSC-iNKT cell therapy. In cases wherein the individual has cancer, the cancer may be primary, metastatic, resistant to therapy, and so forth. patients who have exhausted conventional treatment options.

In particular embodiments, the cells are provided to the patient at 10⁷-10⁹ cells per dose. In specific embodiments, the dosing regimen is a single-dose of allogeneic ^{U}HSC-iNKT cells following lymphodeleting conditioning. The cells may be administered intravenously following lymphodepleting conditioning with fludarabine and cyclophosphamide, for example.

In cases wherein antitumor efficacy in vivo is characterized for subsequent in vivo therapeutic cases, in vivo pharmacological responses may be measured by treating tumor-bearing NSG mice with escalating doses (1×10⁶, 5×10⁶, 10×10⁶) of ^{U}HSC-iNKT cells (n=8 per group); treatment with PBS may be included as a control. Two tumor models may be utilized, as examples. A375.CD1d (1×10⁶ s.c.) may be used as a solid tumor model and MM.1S.Luc (5×10⁶ i.v.) may be used as a hematological malignancy model. Tumor growth can be monitored by either measuring size (A375.CD1d) or bioluminescence imaging (MM.1S.Luc). Antitumor immune responses can be measured by PET imaging, periodic bleeding, and end-point tumor harvest followed by flow cytometry and qPCR. Inhibition of tumor growth in response to ^{U}HSC-iNKT treatment can indicate the therapeutic efficacy of ^{U}HSC-iNKT cell therapy. Correlation of tumor inhibition with iNKT doses can confirm the therapeutic role of the iNKT cells and indicate an effective therapeutic window for human therapy. Detection of iNKT cell responses to tumors can demonstrate the pharmacological antitumor activities of these cells in vivo.

Methods may be employed with respect to individuals who have tested positive for a medical condition, who have one or more symptoms of a medical condition, or who are deemed to be at risk for developing such a condition. In some embodiments, the compositions and methods described herein are used to treat an inflammatory or autoimmune component of a disorder listed herein and/or known in the art.

Certain aspects of the disclosure relate to the treatment of cancer and/or use of cancer antigens. The cancer to be treated or antigen may be an antigen associated with any cancer known in the art or, for example, epithelial cancer, (e.g., breast, gastrointestinal, lung), prostate cancer, bladder cancer, lung (e.g., small cell lung) cancer, colon cancer, ovarian cancer, brain cancer, gastric cancer, renal cell carcinoma, pancreatic cancer, liver cancer, esophageal cancer, head and neck cancer, or a colorectal cancer. In some embodiments, the cancer to be treated or antigen is from one of the following cancers: adenocortical carcinoma, agnogenic myeloid metaplasia, AIDS-related cancers (e.g., AIDS-related lymphoma), anal cancer, appendix cancer, astrocytoma (e.g., cerebellar and cerebral), basal cell carcinoma, bile duct cancer (e.g., extrahepatic), bladder cancer, bone cancer, (osteosarcoma and malignant fibrous histiocytoma), brain tumor (e.g., glioma, brain stem glioma, cerebellar or cerebral astrocytoma (e.g., pilocytic astrocytoma, diffuse astrocytoma, anaplastic (malignant) astrocytoma), malignant glioma, ependymoma, oligodenglioma, meningioma, meningiosarcoma, craniopharyngioma, haemangioblastomas, medulloblastoma, supratentorial primitive neuroectodermal tumors, visual pathway and hypothalamic glioma, and glioblastoma), breast cancer, bronchial adenomas/carcinoids, carcinoid tumor (e.g., gastrointestinal carcinoid tumor), carcinoma of unknown primary, central nervous system lymphoma, cervical cancer, colon cancer, colorectal cancer, chronic myeloproliferative disorders, endometrial cancer (e.g., uterine cancer), ependymoma, esophageal cancer, Ewing's family of tumors, eye cancer (e.g., intraocular melanoma and retinoblastoma), gallbladder cancer, gastric (stomach) cancer, gastrointestinal carcinoid tumor, gastrointestinal stromal tumor (GIST), germ cell tumor, (e.g., extracranial, extragonadal, ovarian), gestational trophoblastic tumor, head and neck cancer, hepatocellular (liver) cancer (e.g., hepatic carcinoma and heptoma), hypopharyngeal cancer, islet cell carcinoma (endocrine pancreas), laryngeal cancer, laryngeal cancer, leukemia, lip and oral cavity cancer, oral cancer, liver cancer, lung cancer (e.g., small cell lung cancer, non-small cell lung cancer, adenocarcinoma of the lung, and squamous carcinoma of the lung), lymphoid neoplasm (e.g., lymphoma), medulloblastoma, ovarian cancer, mesothelioma, metastatic squamous neck cancer, mouth cancer, multiple endocrine neoplasia syndrome, myelodysplastic syndromes, myelodysplastic/myeloproliferative diseases, nasal cavity and paranasal sinus cancer, nasopharyngeal cancer, neuroblastoma, neuroendocrine cancer, oropharyngeal cancer, ovarian cancer (e.g., ovarian epithelial cancer, ovarian germ cell tumor, ovarian low malignant potential tumor), pancreatic cancer, parathyroid cancer, penile cancer, cancer of the peritoneal, pharyngeal cancer, pheochromocytoma, pineoblastoma and supratentorial primitive neuroectodermal tumors, pituitary tumor, pleuropulmonary blastoma, lymphoma, primary central nervous system lymphoma (microglioma), pulmonary lymphangiomyomatosis, rectal cancer, renal cancer, renal pelvis and ureter cancer (transitional cell cancer), rhabdomyosarcoma, salivary gland cancer, skin cancer (e.g., non-melanoma (e.g., squamous cell carcinoma), melanoma, and Merkel cell carcinoma), small intestine cancer, squamous cell cancer, testicular cancer, throat cancer, thymoma and thymic carcinoma, thyroid cancer, tuberous sclerosis, urethral cancer, vaginal cancer, vulvar cancer, Wilms' tumor, and post-transplant lymphoproliferative disorder (PTLD), abnormal vascular proliferation associated with phakomatoses, edema (such as that associated with brain tumors), or Meigs' syndrome.

Certain aspects of the disclosure relate to the treatment of an autoimmune condition and/or use of an autoimmune-associated antigen. The autoimmune disease to be treated or antigen may be an antigen associated with any autoimmune condition known in the art or, for example, diabetes, graft rejection, GVHC, arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, juvenile onset (Type I) diabetes mellitus, including pediatric insulin-dependent diabetes mellitus (IDDM), and adult onset diabetes mellitus (Type II diabetes) and autoimmune diabetes. Also contemplated are immune responses associated with acute and delayed hypersensitivity mediated by cytokines and T-lymphocytes, sarcoidosis, granulomatosis including lymphomatoid granulomatosis, Wegener's granulomatosis, agranulocytosis, vasculitides, including vasculitis, large-vessel vasculitis (including polymyalgia rheumatica and gianT cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), Addison's disease, autoimmune neutropenia, pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, autoimmune or immune-mediated thrombocytopenia such as idiopathic thrombocytopenic purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), experimental autoimmune encephalomyelitis, myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, gianT cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia greata, alopecia totalis, CREST syndrome (calcinosis, Raynaud's phenomenon, esophageal dysmotility, sclerodactyl), and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, gianT cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, asperniogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, graft versus host disease, contact hypersensitivity, asthmatic airway hyperreaction, and endometriosis.

Further aspects relate to the treatment or prevention microbial infection and/or use of microbial antigens. The microbial infection to be treated or prevented or antigen may be an antigen associated with any microbial infection known in the art or, for example, anthrax, cervical cancer (human papillomavirus), diphtheria, hepatitis A, hepatitis B, Haemophilus influenzae type b (Hib), human papillomavirus (HPV), influenza (Flu), japanese encephalitis (JE), lyme disease, measles, meningococcal, monkeypox, mumps, pertussis, pneumococcal, polio, rabies, rotavirus, rubella, shingles (herpes zoster), smallpox, tetanus, typhoid, tuberculosis (TB), varicella (Chickenpox), and yellow fever.

In some embodiments, the methods and compositions may be for vaccinating an individual to prevent a medical condition, such as cancer, inflammation, infection, and so forth.

### VIII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: A Hematopoietic Stem Cell (HSC) Approach to Engineer Off-the-Shelf INKT Cells

The present example concerns generation of off-the-shelf iNKT cells that comprise lack of or down-regulated surface expression of of one or more HLA-I and/or HLA-II molecules. In a specific embodiment, iNKT cells are expanded from healthy donor peripheral blood mononuclear cells (PBMCs), followed by CRISPR-Cas9 engineering to knockout B2M and CIITA genes. Because of the high-variability and low-frequency of iNKT cells in human population (0.001-0.1% in blood), it is beneficial to produce methods that allow alternative means to obtaining iNKT cells.

The present disclosure provides a powerful method to generate iNKT cells from hematopoietic stem cells (HSCs) through genetically engineering HSCs with an iNKT TCR gene and programming these HSCs to develop into iNKT cells (Smith et al., 2015). This method takes advantage of two molecular mechanisms governing iNKT cell development: 1) an Allelic Exclusion mechanism that blocks the rearrangement of endogenous TCR genes in the presence of a transgenic iNKT TCR gene, and 2) a TCR Instruction Mechanism that guides the developing T cells down an iNKT lineage path (Smith et al., 2015). The resulting HSC-engineered iNKT (HSC-iNKT) cells are a homogenous “clonal” population that do not express endogenous TCRs. Mouse HSC-iNKT cells have been generated with a potent anti-cancer efficacy of these iNKT cells in a mouse bone marrow transfer and melanoma lung metastasis model (Smith et al., 2015).

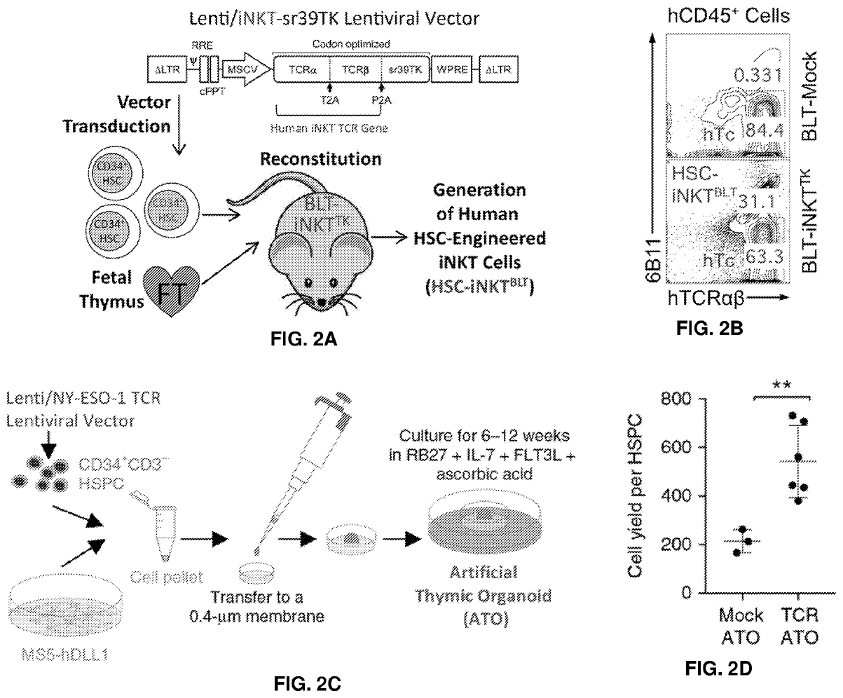

HSC-engineered human iNKT cells are produced by genetically engineering human CD34⁺ peripheral blood stem cells (PBSCs) with a human iNKT TCR gene followed by transferring the engineered PBSCs into a BLT humanized mouse model (FIGS. 2A and 2B). However, such an in vivo approach can only be translated as an autologous HSC adoptive therapy. In particular embodiments, a serum-free, “Artificial Thymic Organoid (ATO)” in vitro culture system that supports the differentiation of TCR-engineered human CD34⁺ HSCs into clonal T cells at high-efficiency and high yield (FIGS. 2C and 2D) (Seet et al., 2017) is utilized. This ATO culture system allows one to move the HSC-iNKT production to an in vitro system, and based on this, an off-the-shelf universal HSC-engineered iNKT (^{U}HSC-iNKT) cell adoptive therapy may be utilized (FIG. 1).

Allogeneic HLA-negative human iNKT cells cultured in vitro from gene-engineered healthy donor HSCs are encompassed herein. Examples of their production are provided below.

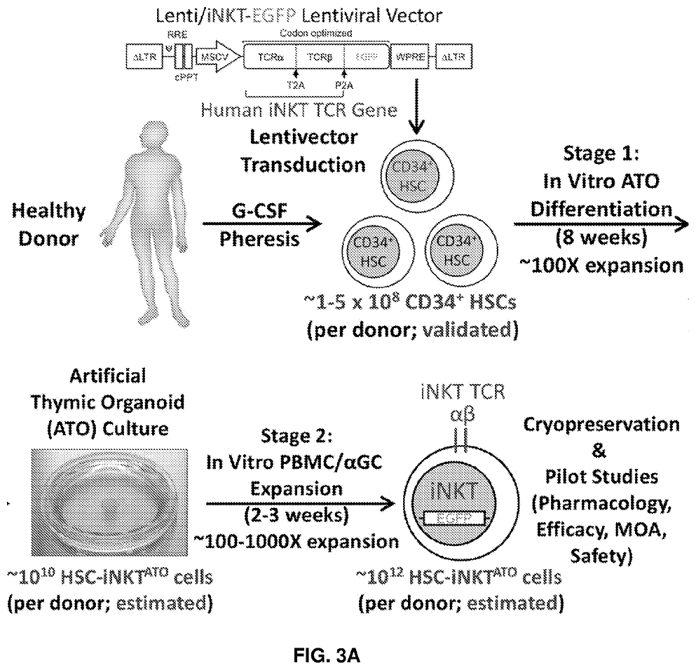

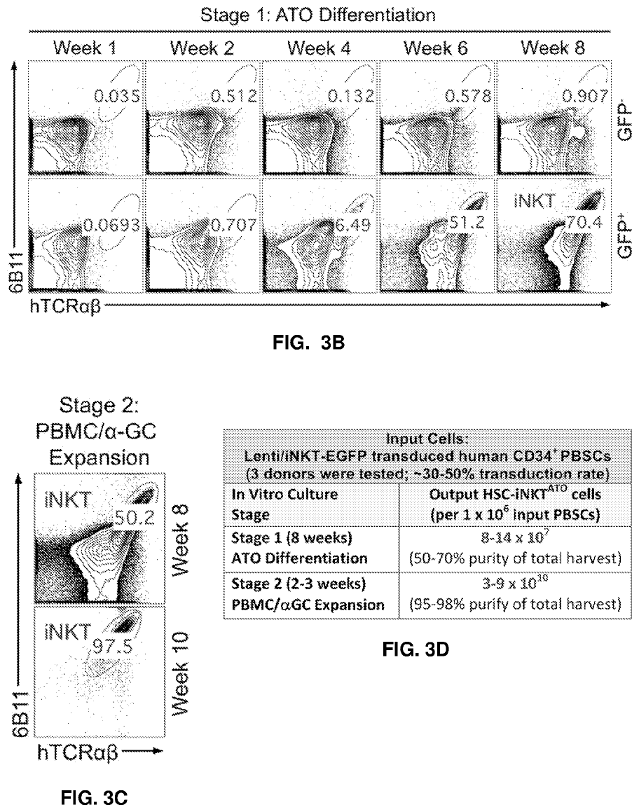

### A. Initial CMC Study (FIG. 3)

Unless otherwise noted, human G-CSF-mobilized peripheral blood CD34⁺ cells contain both hematopoietic stem and progenitor cells. Herein, these CD34⁺ cells are referred to as HSCs.

An initial chemistry, manufacturing, and controls (CMC) study is conducted to test the in vitro manufacture of human HSC-engineered iNKT cells. In specific cases, HSC-iNKT^{ATO} cells are produced, which are HSC-engineered human iNKT cells generated in vitro in a two-stage ATO-□GC culture system.

G-CSF-mobilized human CD34⁺ HSCs were collected from three different healthy donors, transduced with an analog lentiviral vector Lenti/iNKT-EGFP, followed by culturing in vitro in a two-stage ATO-aGC culture system (FIG. 3A). Gene-engineered HSCs (labeled as GFP⁺) efficiently differentiated into human iNKT cells in the Artificial Thymic Organoid (ATO) culture stage over 8 weeks (FIG. 3B), then further expanded in the PBMC/αGC stimulation stage for another 2-3 weeks (FIG. 3C). This manufacturing process was robust and of high yield and high purity for all three donors tested (FIG. 3D). Based on the results, it was estimated that from 1×10⁶ input HSCs (˜30-50% lentivector transduction rate), about 3-9×10¹⁰ HSC-iNKT^{ATO} cells (>95% purity) could be produced, giving a theoretical yield of over 10¹² therapeutic iNKT cells from a single random donor (FIG. 3D).

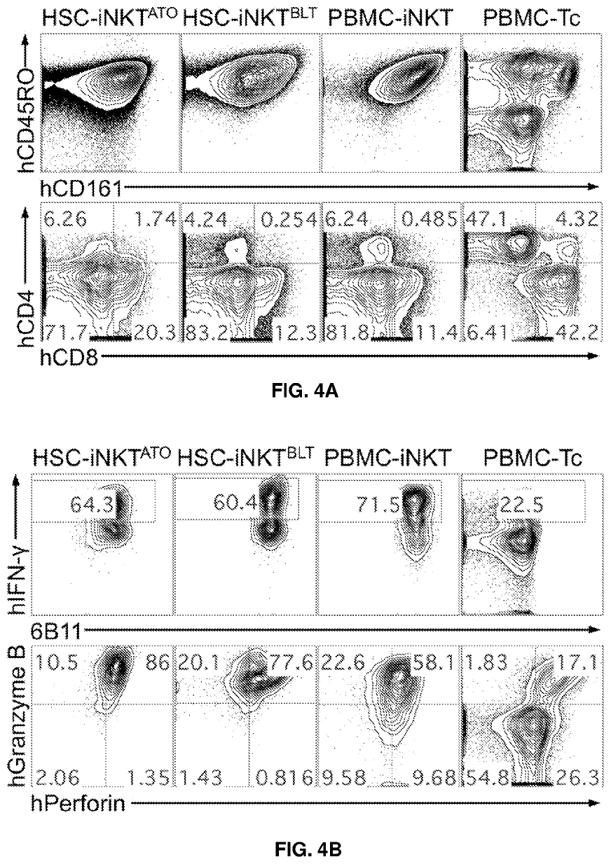

### B. Initial Pharmacology Study (FIG. 4)

An initial pharmacology study was performed to study the phenotype and functionality of human HSC-engineered iNKT cells. The phenotype and functionality of the human HSC-engineered iNKT cells were studied using flow cytometry. Both HSC-iNKT^{ATO} cells (HSC-engineered human iNKT cells generated in vitro in an ATO culture system) and HSC-iNKT^{BLT} cells (HSC-engineered human iNKT cells generated in vivo in a BLT (human bone marrow-liver-thymus engrafted NOD/SCID/γc^{-/-}) humanized mouse model displayed typical iNKT cell phenotype and functionality similar to that of the endogenous PBMC-iNKT cells: they expressed high levels of memory T cell marker CD45RO and NK cell marker CD161 (FIG. 4A); they expressed the CD4 and CD8 co-receptors at a mixed pattern (CD4 single-positive, CD8 single-positive, and CD4/CD8 double-negative) (FIG. 4A); and they produced exceedingly high levels of effector cytokine like IFN-γ and cytotoxic molecules like Perforin and Granzyme B, compared to that of the conventional PBMC-Tc cells (FIG. 4B).

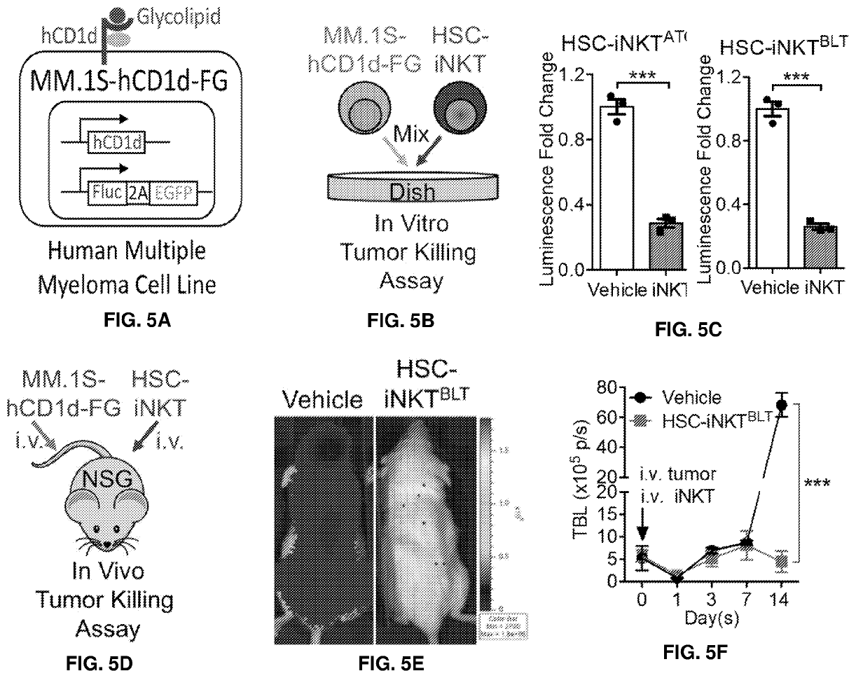

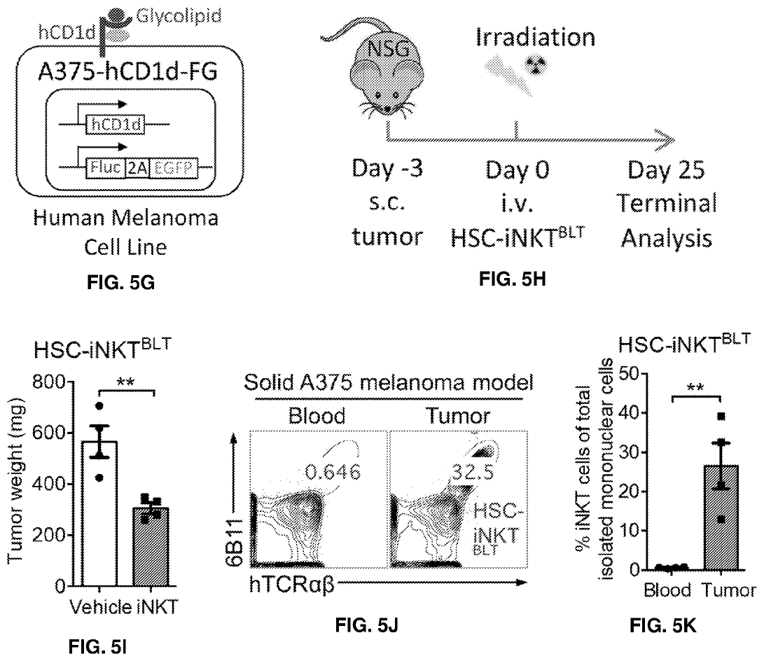

### C. Initial Efficacy Study (FIG. 5)

An initial efficacy study was performed to study the tumor killing efficacy of human HSC-engineered iNKT cells. Human multiple myeloma (MM) cell line MM.1S was engineered to overexpress the human CD1d gene, as well as a firefly luciferase (Flue) reporter gene and an enhanced green fluorescence protein (EGFP) reporter gene (FIG. 5A). The resulting MM.1S-hCD1d-FG cell line was then used to study iNKT cell-targeted tumor killing in vitro in a mixed culture assay (FIG. 5B) and in vivo in an NSG (NOD/SCID/γc^{-/-}) mouse human multiple myeloma (MM) metastasis model (FIG. 5D). Both HSC-iNKT^{ATO} and HSC-iNKT^{BLT} cells showed efficient and comparable tumor killing in vitro (FIG. 5C). HSC-iNKT^{BLT} cells were also tested in vivo and they mediated robust tumor killing (FIGS. 5E and 5F). To study tumor killing efficacy for solid tumors, an A375-hCD1d-FG human melanoma cell line was generated (FIG. 5G). When tested in an NSG mice A375-hCD1d-FG xenograft solid tumor model (FIG. 5H), HSC-iNKT^{BLT} cells efficiently suppressed solid melanoma tumor growth (FIG. 5I). Importantly, HSC-iNKT^{BLT} cells showed targeted infiltration into the tumor sites, presumably due to the potent tumor-trafficking capacity of these cells (FIGS. 5J and 5K).

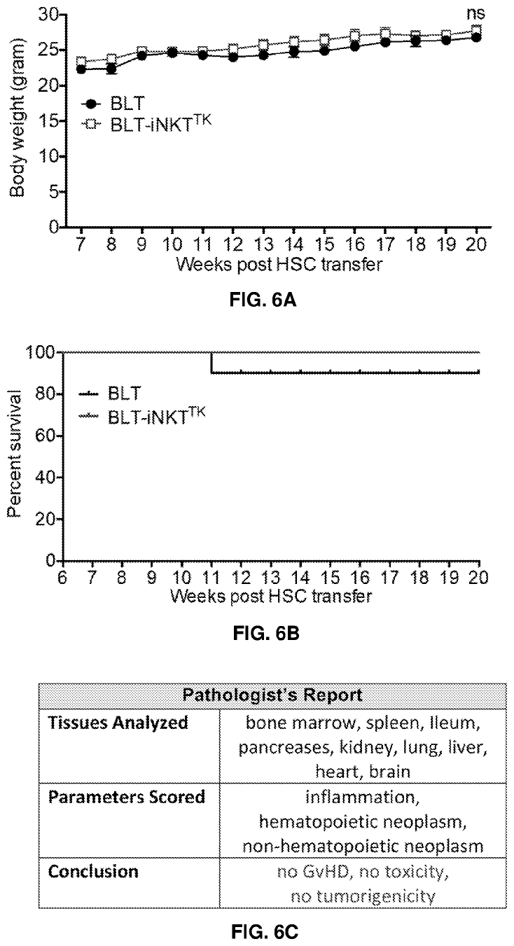

### D. Initial Safety Study—GvHD/Toxicology/Tumorigenicity (FIG. 6)

To access the in vivo long-term GvHD, toxicology, and tumorigenicity of human HSC-engineered iNKT cells, the BLT humanized mice that harbored HSC-iNKT^{BLT} cells were monitored over a period of 5 months post HSC transfer, followed by tissue collection and pathological analysis (FIG. 6). Monitoring of mouse body weight (FIG. 6A), survival (FIG. 6B), and tissue pathology (FIG. 6C) revealed no GvHD, no toxicity, and no tumorigenicity in the BLT-iNKT^{TK} mice (FIG. 2A) compared to the control BLT mice.

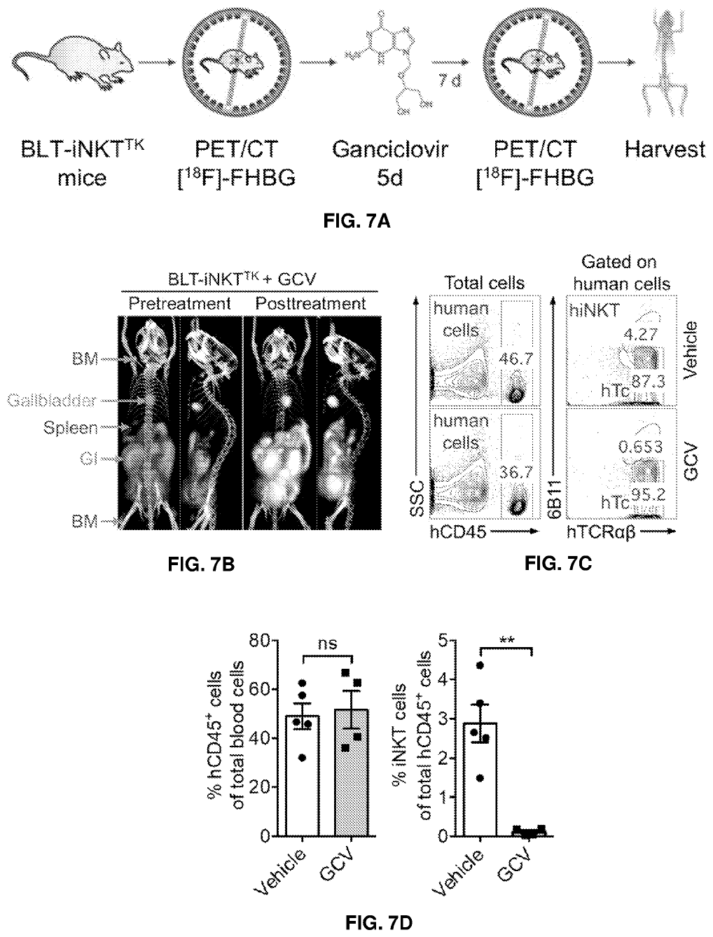

### E. Initial Safety Study—sr39TK Gene for PET Imaging and Safety Control (FIG. 7)

BLT-iNKT^{TK} humanized mice harboring human HSC-engineered iNKT (HSC-iNKT^{BLT}) cells were studied (FIG. 7A). The HSC-iNKT^{BLT} cells were engineered from human HSCs transduced with a Lenti/iNKT-sr39TK lentiviral vector (FIG. 13). Using PET imaging combined with CT scan, we detected the distribution of gene-engineered human cells across the lymphoid tissues of BLT-iNKT^{TK} mice, particularly in bone marrow (BM) and spleen (FIG. 7B). Treating BLT-iNKT^{TK} mice with GCV effectively depleted gene-engineered human cells across the body (FIG. 7B). Importantly, the GCV-induced depletion was specific, evidenced by the selective depletion of the HSC-engineered human iNKT cells but not other human immune cells in BLT-iNKT^{TK} mice as measured by flow cytometry (FIGS. 7C and 7D).

### F. Production of Universal HSC-Engineered iNKT Cells

In specific embodiments, a stem cell-based therapeutic composition is produced that comprises allogeneic HSC-engineered HLA-I/II-negative human iNKT cells (denoted as the Universal HSC-Engineered iNKT cells, ^{U}HSC-iNKT cells).

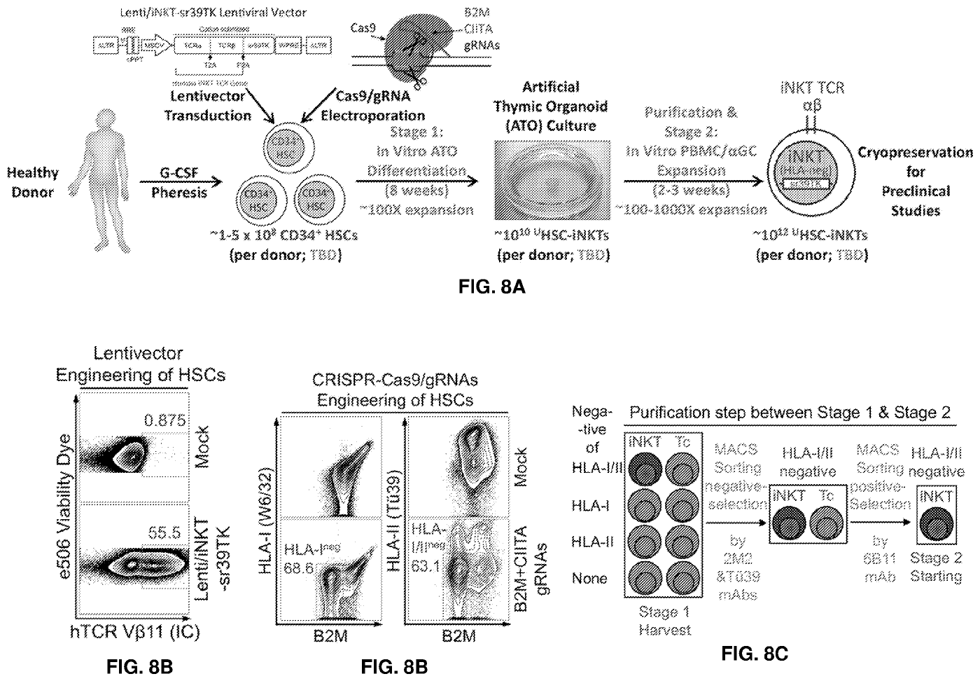

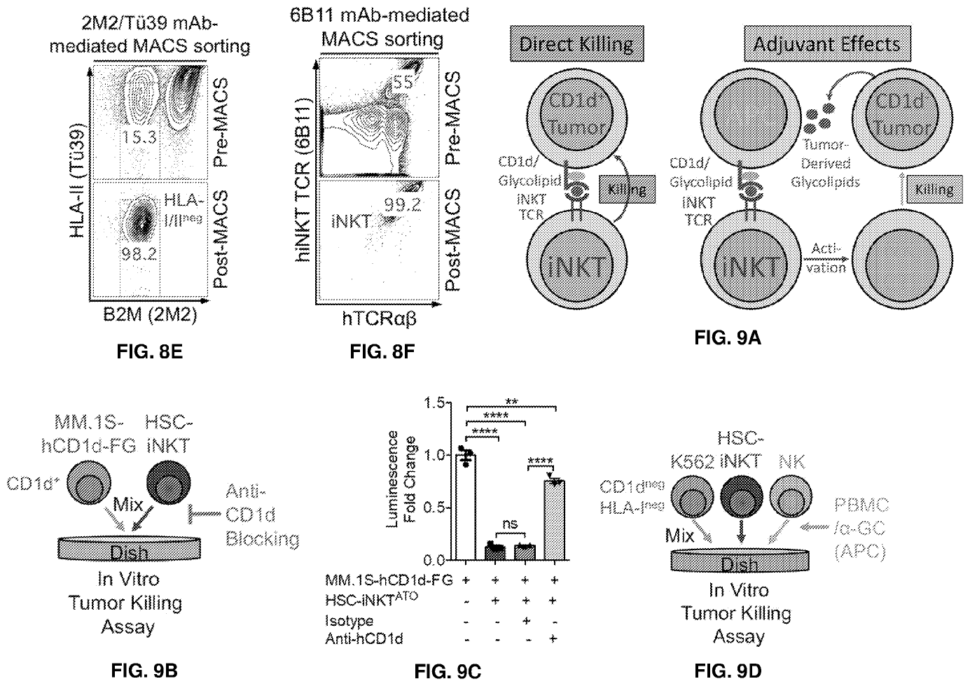

Generate a Lenti-iNKT-sr39TK vector In certain embodiments, a clinical lentiviral vector Lenti/iNKT-sr39TK is utilized (FIG. 8A).

Generate a CRISPR-Cas9/B2M-CITTA-gRNAs complex In specific embodiments, the powerful CRISPR-Cas9/gRNA gene-editing tool is used to disrupt the B2M and CIITA genes in human HSCs (Ren et al., 2017; Liu et al., 2017). iNKT cells derived from such gene-edited HSCs will lack the HLA-I/II expression, thereby avoiding rejection by the host T cells. In an initial study, a CIRSPR-Cas9/B2M-CIITA-gRNAs complex was successfully generated and tested (Cas9 from the UC Berkeley MacroLab Facility; gRNAs from the Synthego; B2M-gRNA sequence 5′-CGCGAGCACAGCUAAGGCCA-3′ (SEQ ID NO:68) (Ren et al., 2017); CIITA-gRNA sequence 5′-GAUAUUGGCAUAAGCCUCCC-3′ (SEQ ID NO:69) (Abrahimi et al., 2015)). To minimize an “off-target” effect, one can utilize the high-fidelity Cas9 protein from IDT (Kohn et al., 2016; Slaymaker et al., 2016; Tsai and Joung, 2016). One can start with the pre-tested single dominant B2M-gRNA and CIITA-gRNA, but in specific embodiments multiple gRNAs are incorporated to further improve gene-editing efficiency.

Collect G-CSF-mobilized CD34⁺ HSCs One can obtain G-CSF-mobilized leukopaks of at least two different healthy donors from a commercial vendor, followed by isolating the CD34⁺ HSCs using a CliniMACS system. After isolation, G-CSF-mobilized CD34⁺ HSCs may be cryopreserved and used later.

Gene-engineer HSCs HSCs may be engineered with both the Lenti-iNKT-sr39TK vector and the CRISPR-Cas9/B2M-CIITA-gRNAs complex. Cryopreserved CD34⁺ HSCs may be thawed and cultured in X-Vivo-15 serum-free medium supplemented with 1% HAS and TPO/FLT3L/SCF for 12 hours in flasks coated with retronectin, followed by addition of the Lenti/iNKT-sr39TK vector for an additional 8 hours (Gschweng et al., 2014). 24 hours post the lentivector transduction, cells may be mixed with pre-formed CIRSPR-Cas9/B2M-CIITA-gRNAs complex and subjected to electroporation using a Lonza Nucleofector. In initial studies, high lentivector transduction rate (>50% transduction rate with VCN=1-3 per cell; FIG. 8B) and high HLA-I/II expression deficiency (˜60% HLA-I/II double-negative cells post a single round of electroporation; FIG. 8C) was achieved using CD34⁺ HSCs from a random donor.

One can further optimize the gene-editing procedure to improve efficiency. Evaluation parameters may include cell viability, deletion (indel) frequency (on-target efficiency) measured by a T7E1 assay and next-generation sequencing (NGS) targeting the B2M and CIITA sites (Tsai et al., 2015), HLA-I/II expression by flow cytometry, and hematopoietic function of edited HSCs measured by the colony formation unit (CFU) assay. One can achieve 30-50% triple-gene editing efficiency of HSCs, which in initial studies could give rise to ˜100 iNKT cells per input HSC post ATO culture (FIG. 3).

Produce ^{U}HSC-iNKT cells One can culture the lentivector and CRISPR-Cas9/gRNA double-engineered HSCs in a 2-stage ATO-aGC in vitro system to produce ^{U}HSC-iNKT cells. At Stage 1, the gene-engineered HSCs will be differentiated into iNKT cells via the Artificial Thymic Organoid (ATO) culture following a standard protocol (FIG. 8A) (Seet et al., 2017). ATO involves pipetting a cell slurry (5 μl) containing a mixture of HSCs (1×10⁴) and irradiated (80 Gy) MS5-hDLL1 stromal cells (1.5×10⁵) as a drop format onto a 0.4-μm Millicell transwell insert, followed by placing the insert into a 6-well plate containing 1 ml RB27 medium (Seet et al., 2017); medium will be changed every 4 days for 8 weeks (Seet et al., 2017). The total harvest from the Stage 1 are expected to contain a mixture of cells. One can perform a purification step to purify the ^{U}HSC-iNKT cells through MACS sorting (2M2/Tü39 mAb-mediated negative selection followed by 6B11 mAb-mediated positive selection) (FIG. 8D). Initial studies showing the effectiveness of this MACS sorting strategy (FIGS. 8E and 8F) are completed. The purified ^{U}HSC-iNKT cells then enter the Stage 2 culture, stimulated with αGC loaded onto irradiated matched-donor CD34⁻ PBMCs (as APCs) and with the supplement of IL-7 and IL-15 (FIG. 8A). Based on initial studies (FIG. 3), ˜10¹⁰ scale of ^{U}HSC-iNKT cells (>99% purity) may be produced from every 1×10⁶ starting HSCs, that will give ˜10¹² pure and homogenous ^{U}HSC-iNKT cellular product from HSCs of a single random donor (FIG. 8A). The resulting ^{U}HSC-iNKT cells may then be cryopreserved and ready for preclinical characterizations.

### G. Characterization of the ^{U}HSC-iNKT Cells

Identity/activity/purity One can study the purity, phenotype, and functionality of the ^{U}HSC-iNKT cell product using pre-established flow cytometry assays (FIG. 4). In specific cases, >99% purity of ^{U}HSC-iNKT cells (gated as hTCRαβ⁺6B11⁺HLA-I/II^{neg}) is acheived. In specific embodiments, these ^{U}HSC-iNKT cells display a typical iNKT cell phenotype (hCD45RO^{hi}hCD161^{hi}hCD4^{+/-}hCD8^{+/-}), express no detectable endogenous TCRs due to allelic exclusion (Seet et al., 2017; Smith et al., 2015; Giannoni et al., 2013), and respond to PBMC/αGC stimulation by producing excess amount of effector cytokines (IFN-7) and cytotoxic molecules (Granzyme B, perform) (FIG. 4) (Watarai et al., 2008).

Pharmacokinetics/pharmacodynamics (PK/PD) One can study the bio-distribution and in vivo dynamics of the ^{U}HSC-iNKT cells by adoptively transferring these cells into tumor-bearing NSG mice. A pre-established A375 human melanoma solid tumor xenograft model may be used (FIG. 5H), for example. Flow cytometry analysis may be performed to study the presence of ^{U}HSC-iNKT cells in tissues. PET imaging may be performed to study the whole-body distribution of ^{U}HSC-iNKT cells, following established protocols (FIG. 7). Based on initial studies, in specific embodiments the ^{U}HSC-iNKT cells can persist in tumor-bearing animals for some time post adoptive transfer, can home to the lymphoid organs (spleen and bone marrow), and most importantly, and can traffic to and infiltrate into solid tumors (FIGS. 5I-5K).

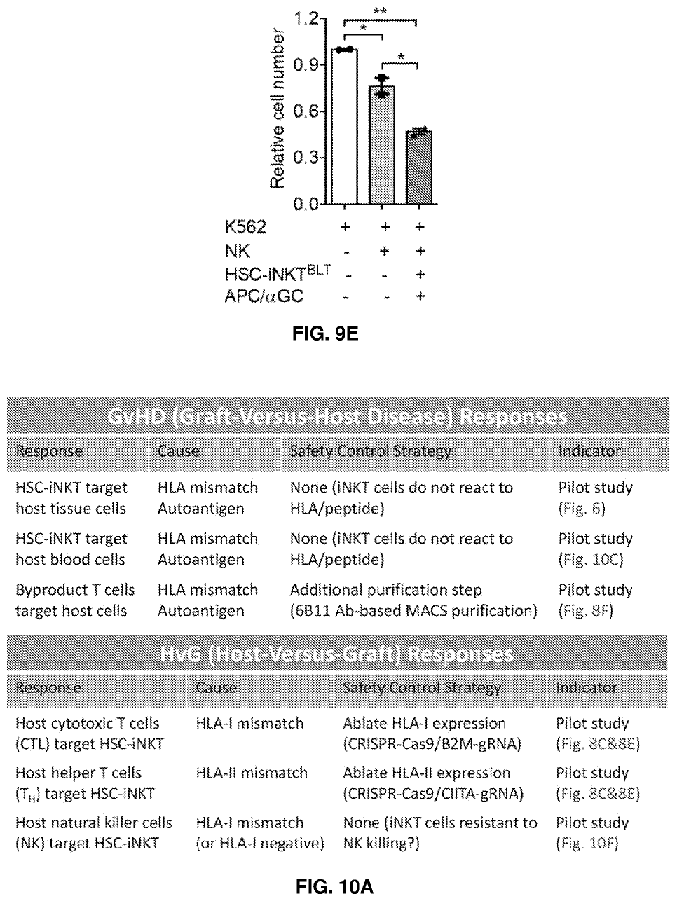

Mechanism of action (MOA) iNKT cells can target tumor through multiple mechanisms: 1) they can directly kill CD1d⁺ tumor cells through iNKT TCR stimulation, and 2) they can indirectly target CD1d⁻ tumor cells through recognizing tumor-derived glycolipids presented by tumor-associated antigen-presenting cells (which constantly express CD1d), then activating the downstream effector cells, like NK cells and CTLs, to kill these CD1d⁻ tumor cells (FIG. 9A) (Vivier et al., 2012). Many cancer cells produce glycolipids that can stimulate iNKT cells, albeit the nature of such “altered” glycolipids remain to be elucidated (Bendelac et al., 2007). Using an in vitro direct tumor killing assay (FIG. 9B), the therapeutic surrogates HSC-iNKT^{ATO} and HSC-iNKT^{BLT} cells directly killed tumor cells in an CD1d/TCR-dependent manner (FIG. 9C). Using an in vitro mixed culture assay (FIG. 9D), it was further shown that HSC-iNKT^{BLT} cells stimulated by APCs could activate NK cells to kill CD1d⁻HLA-I^{-/-} K562 human myeloid leukemia cells (FIG. 9E). These pre-established assays may be utilized to study ^{U}HSC-iNKT cell targeting of tumor cells. In particular embodiments, the ^{U}HSC-iNKT cells can target tumor through both direct killing and adjuvant effects.

Efficacy One can study the tumor killing efficacy of ^{U}HSC-iNKT cells using the pre-established in vitro and in vivo assays (FIG. 5). Both a human blood cancer model (MM1.S multiple myeloma) and a human solid tumor model (A375 melanoma) may be used (FIG. 5), for example. In certain embodiments, the ^{U}HSC-iNKT cells can effectively kill both MM1.S and A375 tumor cells in vitro and in vivo, similar to what has been observed for the therapeutic surrogates HSC-iNKT^{ATO} and HSC-iNKT^{BLT} cells (FIG. 5).

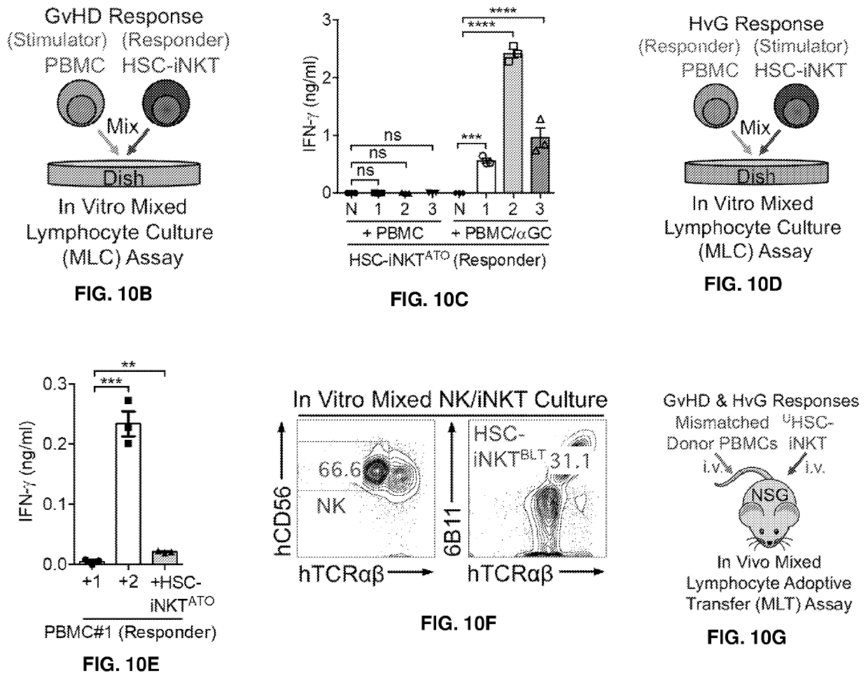

Safety One can study the safety of ^{U}HSC-iNKT adoptive therapy on three aspects, as example: a) general toxicity/tumorigenicity, b) immunogenicity, and c) suicide gene “kill switch”. 1) The long-term GvHD (against recipient animal tissues), toxicology, and tumorigenicity of ^{U}HSC-iNKT cells may be studied through adoptively transferring these cells into NS G mice and monitoring the recipient mice over a period of 20 weeks ended by terminal pathology analysis, following an established protocol (FIG. 6). No GvHD, no toxicity, and no tumorigenicity are expected (FIG. 6). 2) For immune cell-based adoptive therapies, there are always two immunogenicity concerns: a) Graft-Versus-Host Disease (GvHD) responses, and b) Host-Versus-Graft (HvG) responses. Engineered safety control strategies mitigate the possible GvHD and HvG risks for the ^{U}HSC-iNKT cellular product (FIG. 10A). Possible GvHD and HvG responses are studied using an established in vitro Mixed Lymphocyte Culture (MLC) assay (FIGS. 10B and 10D) and an in vivo Mixed Lymphocyte Adoptive Transfer (MLT) Assay (FIG. 10G). The readouts of the in vitro MLC assays may be IFN-γ production analyzed by ELISA, while the readouts of the in vivo MLT assays may be the elimination of targeted cells analyzed by bleeding and flow cytometry (either the killing of mismatched-donor PBMCs as a measurement of GvHD response, or the killing of ^{U}HSC-iNKT cells as a measurement of HvG response). Based on initial studies, in specific embodiments the ^{U}HSC-iNKT cells do not induce GvHD response against host animal tissues (FIG. 6), and do not induce GvHD response against mismatched-donor PBMCs (FIG. 10C). In specific embodiments, ^{U}HSC-iNKT cells are resistant to HvG-induced elimination. Initial studies showed that even with HLA-I/II expression, HSC-iNKT^{ATO} cells were already weak targets for mismatched-donor PBMC T cells (FIG. 10E). In specific cases there is a total lack of T cell-mediated HvG response against the ^{U}HSC-iNKT cells. Interestingly, initial studies showed that the surrogate HSC-iNKT^{BLT} cells were resistant to killing by mismatched-donor NK cells (FIG. 10F). In some cases, lack of HLA-I expression on ^{U}HSC-iNKT cells may make these cells more susceptible to NK killing. Therefore the final ^{U}HSC-iNKT cellular product may be tested. 3) One can study the elimination of ^{U}HSC-iNKT cells in recipient NSG mice through GCV administration, following an established protocol (FIG. 7). Based on initial studies, the sr39TK suicide gene can function as a potent “kill switch” to eliminate ^{U}HSC-iNKT cells in case of a safety need.

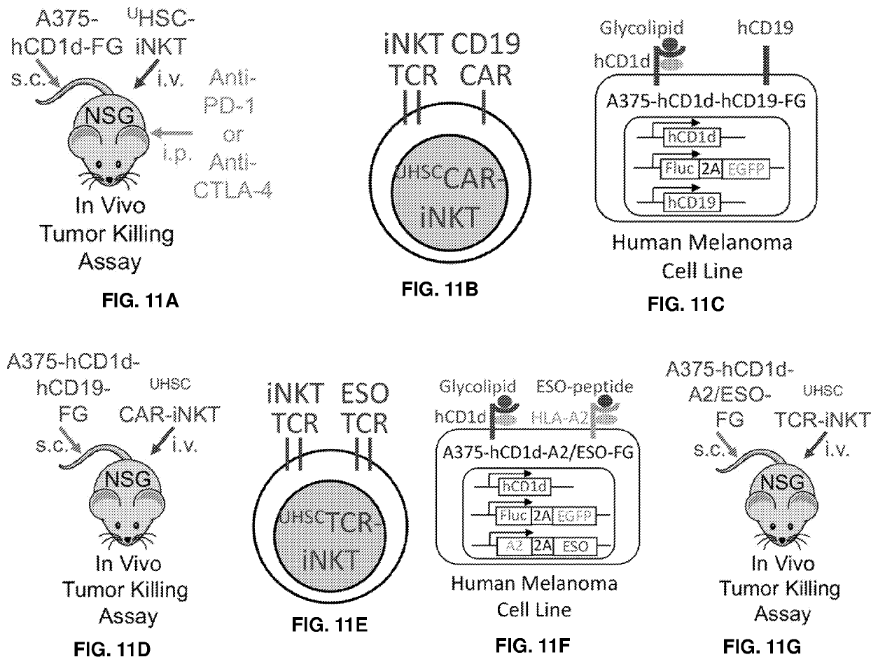

Combination therapy One can examine ^{U}HSC-iNKT cells for combination immunotherapy. In particular, there are synergistic therapeutic effects combining the ^{U}HSC-iNKT adoptive therapy with the checkpoint blockade therapy (e.g., PD-1 and CTLA-4 blockade) (Pilones et al., 2012; Durgan et al., 2011). A pre-established human melanoma solid tumor model (A375-hCD1d-FG) may be used (FIG. 11A). One can further engineer the ^{U}HSC-iNKT cells to express cancer-targeting CARs (chimeric antigen receptors) or TCRs (T cell receptors) for next-generation universal CAR-iNKT and TCR-iNKT therapies (denoted as ^{UHSC}CAR-iNKT and ^{UHSC}TCR-iNKT therapies) (Oberschmidt et al., 2017; Bollino and Webb, 2017; Heczey et al., 2014; Chodon et al., 2014). For the study of ^{UHSC}CAR-iNKT therapy, ^{U}HSC-iNKT cells may be transduced with a lentivector encoding a CD19-CAR gene (FIG. 11B). Meanwhile, the human melanoma cell line A375-hCD1d-FG, as an example, may be further engineered to overexpress the human CD19 antigen (FIG. 11C). The anti-tumor efficacy of the ^{UHSC}CAR-iNKT cells may be studied using the A375-hCD1d-hCD19-FG tumor xenograft model (FIG. 11D). For the study of ^{UHSC}TCR-iNKT therapy, ^{U}HSC-iNKT cells may be transduced with a lentivector encoding an NY-ESO-1 TCR gene (FIG. 11E). The A375-hCD1d-FG cell line may be further engineered to overexpress the human HLA-A2 molecule and the NY-ESO-1 antigen (FIG. 11F). The anti-tumor efficacy of the ^{UHSC}TCR-iNKT cells may be studied using the A375-hCD1d-A2/ESO-FG tumor xenograft model (FIG. 11G).

### H. Pharmacology Embodiments

Drug mechanism for ^{U}HSC-iNKT therapy ^{U}HSC-iNKT is a cellular product that at least in some cases is generated by 1) genetic modification of donor HSCs to express iNKT TCRs via lentiviral vectors and to knockout HLAs via CRISPR/Cas9-based gene editing, 2) in vitro differentiation into iNKT cells via an ATO culture, 3) in vitro iNKT cell expansion, and 4) formulation and cryopreservation. Once infused into patients, this cell product can employ multiple mechanisms to target and eradicate tumor cells, in at least some embodiments. The infused cells can directly recognize and kill CD1d⁺ tumor cells through cytotoxicity. They can secrete cytokines such as IFN-γ to activate NK cells to kill HLA-negative tumor cells, and also activate DCs which then stimulate cytotoxic T cells to kill HLA-positive tumor cells. Accordingly, a series of in vitro and in vivo studies may be utilized to demonstrate the pharmacological efficacy of this cell product for cancer therapy.

In vitro surface and functional characterization An efficient protocol to generate ^{U}HSC-iNKT cells is provided herein. An efficient gene editing of HSCs to ablate the expression of class I HLA via knockout of B2M is also demonstrated. Taking advantage of the multiplex editing CRISPR/Cas9, one can also simultaneously disrupt class II HLA expression via knockout of the gene for the class II transactivator (CIITA), a key regulator of HLA-II expression (Steimle et al., 1994), using a validated gRNA sequence (Abrahimi et al., 2015). Thus, incorporating this gene editing step to disrupt HLA-I and HLA-II expression and the microbeads purification step, one can generate ^{U}HSC-iNKT cells (details provided elsewhere herein). Flow cytometric analysis may be used to measure the purity and the surface phenotypes of these engineered iNKT cells. The cell purity may be characterized by TCR Vα24-Jα18(6B11)⁺HLA-I/II^{neg}. In at least some cases, this iNKT cell population should be CD45RO⁺CD161⁺, indicative of memory and NK phenotypes, and contain CD4⁺CD8⁻ (CD4 single-positive), CD4⁻CD8⁺ (CD8 single-positive), and CD4⁻CD8⁻ (double-genative, DN)(Kronenberg and Gapin, 2002). One can analyze CD62L expression, as a recent study indicated that its expression is associated with in vivo persistence of iNKT cells and their antitumor activity (Tian et al., 2016). One can compare these phenotypes of ^{U}HSC-iNKT with that iNKT from PBMCs. RNAseq may be employed to perform comparative gene expression analysis on ^{U}HSC-iNKT and PBMC iNKT cells.

IFN-γ production and cytotoxicity assays may be used to assess the functional properties of ^{U}HSC-iNKT, using PBMC iNKT as the benchmark control. ^{U}HSC-iNKT cells may be simulated with irradiated PBMCs that have been pulsed with αGalCer and supernatants harvested from one day stimulation will be subjected to IFN-γ ELISA (Smith et al., 2015). Intracellular cytokine staining (ICCS) of IFN-γ may be performed as well on iNKT cells after 6-hour stimulation. The cytotoxicity assay may be conducted by incubating effector ^{U}HSC-iNKT cells with aGC-loaded A375.CD1d target cells engineered to expression luciferase and GFP for 4 hours and cytotoxicity may be measured by a plate reader for its luminescence intensity. Because sr39TK is introduced as a PET/suicide gene, one can verify its function by incubating ^{U}HSC-iNKT with ganciclovir (GCV) and cell survival rate may be measured by a MTT assay and an Annexin V-based flow cytometric assay.

Pharmacokinetics/Pharmacodynamics (PK/PD) studies The PK/PD studies may determine in vivo in animal models: 1) expansion kinetics and persistence of infused ^{U}HSC-iNKT; 2) biodistribution of ^{U}HSC-iNKT in various tissues/organs; 3) ability of ^{U}HSC-iNKT to traffic to tumors and how this filtration relates to tumor growth. Immunodeficient NSG mice bearing A375.CD1d (A375.CD1d) tumors may be utilized as the solid tumor animal model. The study design is outlined in FIG. 11. Two examples of cell dose groups (1×10⁶ and 10×10⁶; n=8) may be investigated. The tumors are inoculated (s.c.) on day −4 and the baseline PET imaging and bleeding is conducted on day 0. Subsequently, ^{U}HSC-iNKT cells is infused intravenously (i.v.) and monitored by 1) PET imaging in live animals on days 7 and 21; 2) periodic bleeding on days 7, 14 and 21; 3) end-point tissue collection after animal termination on day 21. Cell collected from various bleedings may be analyzed by flow cytometry; iNKT cells are TCRαβ⁺6B11⁺, in specific embodiments. One can examine the expression of other markers such as CD45RO, CD161, CD62L, and CD4/CD8 to see how iNKT subsets vary over the time. PET imaging via sr39TK will allow tracking of the presence of iNKT cells in tumors and other tissues/organs such as bone, liver, spleen, thymus, etc. At the end of the study, tumors and mouse tissues including spleen, liver, brain, heart, kidney, lung, stomach, bone marrow, ovary, intestine, etc., are harvested for qPCR analysis to examine the distribution of ^{U}HSC-iNKT cells.

Antitumor efficacy in vivo In vivo pharmacological responses are measured by treating tumor-bearing NSG mice with escalating doses (1×10⁶, 5×10⁶, 10×10⁶) of ^{U}HSC-iNKT cells (n=8 per group); treatment with PBS is included as a control. Two tumor models may be utilized as examples. A375.CD1d (1×10⁶ s.c.) may be used as a solid tumor model and MM.1S.Luc (5×10⁶ i.v.) may be used as a hematological malignancy model. Tumor growth is monitored by either measuring size (A375.CD1d) or bioluminescence imaging (MM.1S.Luc). Antitumor immune responses are measured by PET imaging, periodic bleeding, and end-point tumor harvest followed by flow cytometry and qPCR. Inhibition of tumor growth in response to ^{U}HSC-iNKT treatment indicates the therapeutic efficacy of proposed ^{U}HSC-iNKT cell therapy. Correlation of tumor inhibition with iNKT doses confirms the therapeutic role of the iNKT cells and can indicate an effective therapeutic window for human therapy. Detection of iNKT cell responses to tumors demonstrates the pharmacological antitumor activities of these cells in vivo.

Mechanism of action (MOA) iNKT cells are known to target tumor cells through either direct killing, or through the massive release of IFN-γ to direct NK and CD8 T cells to eradicate tumors (Fujii et al., 2013). An in vitro pharmacological study provides evidence of direct cytotoxicity. Here one can investigate the possible roles of NK and CD8 T cells in assisting antitumor reactivity in vivo. Tumor-bearing NSG mice (A375.CD1d or MM.1S.Luc) may be infused with either ^{U}HSC-iNKT alone (a dose chosen based on above in vivo study) or in combination with PBMCs (mismatched donor, 5×10⁶); owing to the MHC negativity of ^{U}HSC-iNKT, no allogenic immune response is expected between ^{U}HSC-iNKT and unrelated PBMCs. Tumor growth may be monitored and compared between with and without PBMC groups (n=8 per group). If a greater antitumor response is observed from the combination group, it will indicate that at least in specific embodiments components in PBMCs, presumably NK and/or CD8 T cells, play a role to boost therapeutic efficacy. To further determine their individual roles, PBMCs with depletion of NK (via CD56 beads), CD8 T cells (via CD8 beads), or myeloid (via CD14 beads) cells, are co-infused along with ^{U}HSC-iNKT cells into tumor-bearing mice. Immune checkpoint inhibitors such as PD-1 and CTLA-4 have been suggested to regulate iNKT cell function (Pilones et al., 2012; Durgan et al., 2011). Through adding anti-PD-1 or anti-CTLA-4 treatment to the ^{U}HSC-iNKT therapy, one can understand how these molecules modulate ^{U}HSC-iNKT therapy and provide valuable guidance on the design of combination cancer therapy, for example.

### I. Embodiments of Chemistry, Manufacturing and Controls

CMC overview In certain embodiments, the manufacturing of ^{U}HSC-iNKT involves: 1) collection of G-CSF-mobilized leukopak; 2) purification of GCSF-leukopak into CD34⁺ HSCs; 3) transduction of HSCs with lentiviral vector Lenti/iNKT-sr39TK; 4) gene editing of B2M and CIITA via CRISPR/Cas9; 5) in vitro differentiation into iNKT cells via ATO; 6) purification of iNKT cells; 7) in vitro cell expansion; 8) cell collection, formulation and cryopreservation (FIG. 14). As examples, there are two drug substances (Lenti/iNKT-sr39TK vector and ^{U}HSC-iNKT cells), and the final drug product is the formulated and cryopreserved ^{U}HSC-iNKT in infusion bags, in at least some cases.

1. Vector Manufacturing

Vector structure One vector for genetic engineering of HSCs into iNKT cells is an HIV-1 derived lentiviral vector Lenti/iNKT-sr39TK encoding a human iNKT TCR gene along with an sr39TK PET imaging/suicide gene (FIG. 13). The key components of this third generation self-inactivating (SIN) vector are: 1) 3′ self-inactivating long-term repeats (ΔLTR); 2) Ψ region vector genome packaging signal; 3) Rev Responsive Element (RRE) to enhance nuclear export of unspliced vector RNA; 4) central PolyPurine Tract (cPPT) to facilitate unclear import of vector genomes; 5) expression cassette of the α chain gene (TCRα) and β chain gene (TCRβ) of a human iNKT TCR, as well as the PET/suicide gene sr39TK (Gschweng et al., 2014) driven by internal promoter from the murine stem cell virus (MSCV). The iNKT TCRα and TCRβ and sr39TK genes are all codon-optimized and linked by 2A self-cleaving sequences (T2A and P2A) to achieve their optimal co-expression (Gschweng et al., 2014).

Quality control of vector A series of QC assays may be performed to ensure that the vector product is of high quality. Those standard assays such as vector identity, vector physical titer, and vector purity (sterility, mycoplasma, viral contaminants, replication-competent lentivirus (RCL) testing, endotoxin, residual DNA and benzonase) is conducted at IU VPF and provided in the Certificate of Analysis (COA). Additional QC assays one can perform include 1) the transduction/biological titer (by transducing HT29 cells with serial dilutions and performing ddPCR, ≥1×10⁶ TU/ml); 2) the vector provirus integrity (by sequencing the vector-integrated portion of genomic DNA of transduced HT29 cells, same to original vector plasmid sequence); 3) the vector function. The vector function maybe measured by transducing human PBMC T cells (Chodon et al., 2014). The expression of iNKT TCR gene may be detected by staining with the 6B11 specific for iNKT TCR (Montoya et al., 2007). The functionality of expressed iNKT TCRs may be analyzed by IFN-γ production in response to αGalCer stimulation (Watarai et al., 2008). The expression and functionality of sr39TK gene may be analyzed by penciclovir update assay and GCV killing assay (Gschweng et al., 2014). The stability of the vector stock (stored in −80 freezer) may be tested every 3 months by measuring its transduction titer. These QC assays may be validated.

2. Cell Manufacturing and Product Formulation

Overview of manufacturing uHSC-iNKT cells ^{U}HSC-iNKT cells are one embodiment of a drug substance that will function as “living drug” to target and fight tumor cells. They are generated by in vitro differentiation and expansion of genetically modified donor HSCs. Initial data demonstrate a novel and efficient protocol to produce them in a laboratory scale. In order to make them as an “off-the-shelf” cell product, one can develop and validate a GMP-comparable manufacturing process. As an example, target of production scale is 10¹² cells per batch, which is estimated to treat 1000-10,000 patients.

Cell manufacturing process One embodiment of a cell manufacturing process is outlined in FIG. 13, with defined timelines and key “In-Process-Control (IPC)” measurements for each process step. Step 1 is to harvest donor G-CSF-mobilized PBSCs in blood collection facilities, which has become a routine procedure in many hospitals (Deotare et al., 2015). One can obtain fresh PBSCs in Leukopaks from the HemaCare for this project; HemaCare has IRB-approved collection protocols and donor consents and can support clinical trials and commercial product manufacturing (A Support Letter from Hemacare is included in the Application). Step 2 is to enrich CD34⁺ HSCs from PBSCs using a CliniMACS system; one can use such a system located at the UCLA GMP facility to complete this step and expect to yield at least 10⁸ CD34⁺ cells. CD34⁻ cells are collected and stored as well (may be used as PBMC feeder in Step 7).

Step 3 involves the HSC culture and vector transduction. CD34⁺ cells are cultured in X-VIVO15 medium supplemented with 1% HAS (USP) and growth factor cocktails (c-kit ligand, flt-3 ligand and tpo; 50 ng/ml each) for 12 hrs in flasks coated with retronectin, followed by addition of the Lenti/iNKT-sr39TK vector for additional 8 hrs (Gschweng et al., 2014). Vector integration copies (VCN) are measured by sampling ˜50 colonies formed in the methylcellulose assay for transduced cells and one can determine the average vector copy number per cell using ddPCR (Nolta et al., 1994). One can routinely achieved >50% transduction with VCN=1-3 per cell, in at least some cases.

Step 4 is to utilize the powerful CRISPR/Cas9 multiplex gene editing method to target the genomic loci of both B2M and CIITA in HSCs and disrupt their gene expression (Ren et al., 2017; Liu et al., 2017), and iNKT cells derived from edited HSCs will lack the MHC/HLA expression, thereby avoiding the rejection by the host immune system. Initial data has demonstrated the success of the B2M disruption for CD34⁺ HSCs with high efficiency (˜75% by flow analysis) via electroporation of Cas9/B2M-gRNA. B2M/CIITA double knockout may be achieved by electroporation of a mixture of RNPs (Cas9/B2M-gRNA and Cas9/CIITA-gRNA (Abrahimi et al., 2015)). One can optimize and validate this process (Gundry et al., 2016) by varying electroporation parameters, ratios of two RNPs, stem cell culture time (24, 48, or 72 hrs post-transduction) prior to electroporation, etc; one can use the high fidelity Cas9 protein (Slaymaker et al., 2016; Tsai and Joung, 2016) from IDT to minimize the “off-target” effect. Evaluation parameters may be viability, deletion (indel) frequency (on-target efficiency) measured by a T7E1 assay and next-generation sequencing (NGS) targeting the B2M and CIITA sites, MHC expression by flow cytometry, and hematopoietic function of edited HSCs measured by the colony formation unit (CFU) assay, for example.

Step 5 is to in vitro differentiate modified CD34⁺ HSCs into iNKT cells via the artificial thymic organoid (ATO) culture (Seet et al., 2017). Initial studies have shown that functional iNKT cells can be efficiently generated from HSCs engineered to express iNKT TCRs. Building upon this data, one can test and validate an 8-week, GMP-compatible ATO culture process to produce 10¹⁰ iNKT cells from 10⁸ modified CD34⁺ HSCs. ATO involves pipetting a cell slurry (5 μl) containing mixture of HSCs (5×10⁴) and irradiated (80 Gy) MS5-hDLL1 stromal cells (10⁶) as a drop format onto a 0.4-μm Millicell transwell insert, followed by placing the insert into a 6-well plate containing 1 ml RB27 medium (Seet et al., 2017); medium can be changed every 4 days for 8 weeks. Considering 3 ATOs per insert, one may need approximately 170 six-well plates for each batch production. An automated programmable pipetting/dispensing system (epMontion 5070f from Eppendorf) placed in biosafety cabinet for plating ATO droplets and medium exchange may be used; a 2-hr operation may be needed for completing 170 plates each round. At the end of ATO culture, iNKT cells are harvested and characterized. As one example, a component of ATO is the MS5-hDLL1 stromal cell line that is constructed by lentiviral transduction to express human DLL1 followed by cell sorting. In preparation for one embodiment of the GMP process, one can perform a single cell clonal selection process on this polyclonal cell population to establish several clonal MS5-hDLL1 cell lines, from which one can choose an efficient one (evaluated by ATO culture) and use it to generate a master cell bank. Once certified, this bank may be used to supply irradiated stromal cells for future clinical grade ATO culture.

Step 6 is to purify ATO-derived iNKT cells using the CliniMACS system. This step purification is to deplete MHCl⁺ and MHCII⁺ cells and enrich iNKT⁺ cells. Anti-MHCI and anti-MHCII beads may be prepared by incubating Miltenyi anti-Biotin beads with commercially available biotinylated anti-B2M (clone 2M2), anti-MHCI (clone W6/32, HLA-A, B, C), anti-MHCII (clone Tu39, HLA-DR, DP, DQ), and anti-TCR Vα24-Jα18 (clone 6B11) antibodies; microbeads directly coated with 6B11 antibodies are also are available from Miltenyi Biotec. Harvested iNKT cells are labeled by anti-MHC bead mixtures and washed twice and MHCI⁺ and/or MHCII⁺ cells are depleted using the CliniMACS depletion program; if necessary, this depletion step can be repeated to further remove residual MHC⁺ cells. Subsequently, iNKT cells are further purified using the standard anti-iNKT beads and the CliniMACS enrichment program. The cell purity may be measured by flow cytometry.

Step 7 is to expand purified iNKT cells in vitro. Starting from 10¹⁰ cells, one can expand into 10¹² iNKT cells using an already validated PBMC feeder-based in vitro expansion protocol (Yamasaki et al., 2011; Heczey et al., 2014). One can evaluate a G-Rex-based bioprocess for this cell expansion. G-Rex is a cell growth flask with a gas-permeable membrane at the bottom allowing more efficient gas exchange; A G-Rex500M flask has the capacity to support a 100-fold cell expansion in 10 days (Vera et al., 2010; Bajgain et al., 2014; Jin et al., 2012). The stored CD34⁻ cells (used as feeder cells) from the Step 1 are thawed, pulsed with αGalCer (100 ng/ml), and irradiated (40 Gy). iNKT cells will be mixed with irradiated feeder cells (1:4 ratio), seeded into G-Rex flasks (1.25×10⁸ iNKT each, 80 flasks), and allowed to expand for 2 weeks. IL-2 (200 U/ml) will be added every 2-3 days and one medium exchange will occur at day 7; all medium manipulation may be achieved by peristaltic pumps. This expansion process should be GMP-compatible because a similar PBMC feeder-based expansion procedure (termed rapid expansion protocol) has been already utilized to produce therapeutic T cells for many clinical trials Dudley et al., 2008; Rosenberg et al., 2008).

Step 8 is to formulate the harvested iNKT cells from Step 7 (the active drug component) into cell suspension for direct infusion. After at least 3 rounds of extensive washing, cells from Step 7 may be counted and suspended into an infusion/cold storage-compatible solution (10⁷-10⁸ cells/ml), which is composed of Plasma-Lyte A Injection (31.25% v/v), Dextrose and Sodium Chloride Injection (31.25% v/v), Human Albumin (20% v/v), Dextran 40 in Dextrose Inject (10%, v/v) and Cryosery DMSO (7.5%, v/v); this solution has been used to formulate tisagenlecleucel, an approved T cell product from Novartis (Grupp et al., 2013). Once filled into FDA-approved freezing bags (such as CryoMACS freezing bags from Miltenyi Biotec), the product may be frozen in a controlled rate freezer and stored in a liquid nitrogen freezer. One can perform validation and/or optimization studies by measuring viability and recovery to ensure that this formulation is appropriate for our ^{U}HSC-iNKT cell product.

Quality control for bioprocessing and product Various IPC assays such as cell counting, viability, sterility, mycoplasma, identity, purity, VCN, etc.) may be incorporated into the proposed bioprocess to ensure a high-quality production. The proposed product releasing testing include 1) appearance (color, opacity); 2) cell viability and count; 3) identity and VCN by qPCR for iNKT TCR; 4) purity by iNKT positivity and B2M negativity; 5) endotoxins; 6) sterility; 7) mycoplasma; 8) potency measured by IFN-γ release in response to αGalCer stimulation; 9) RCL (replication-competent lentivirus) (Cornetta et al., 2011). Most of these assays are either standard biological assays or specific assays unique to this product that may be validated. Product stability testing may be performed by periodically thawing LN-stored bags and measuring their cell viability, purity, recovery, potency (IFN-γ release) and sterility. In particular embodiments, the product is stable for at least one year.

### J. Safety Embodiments

Tumorigenecity in vitro and in vivo and acute toxicity in vivo One can evaluate the potential of ^{U}HSC-iNKT cells for transformation or autonomous proliferation. The in vitro assays include 1) G-banded karyotyping, which may be conducted on αGalCer-restimulated, actively dividing ^{U}HSC-iNKT cells to determine whether a normal karyotype is maintained; 2) homeostatic proliferation (without stimulation) of the cell product, which may be measured by flow cytometric analysis of the dilution of cell-labeled PKH dyes (the αGalCer-stimulated cell group will be used as a proliferation-positive control) (Hurton et al., 2016); 3) the soft agar colony formation assay (Horibata et al., 2015), which may be employed to evaluate the anchorage-independent growth capacity of the iNKT cell product. NSG naïve mice infused with 10⁷ iNKT cells may be used to examine the in vivo tumorigenecity and long-term toxicity (4-6 months, n=6) by analyzing various harvested tissues/organs for any abnormality and by measuring the presence of iNKT cells in blood, spleen, bone marrow and liver for any aberrant proliferation (Hurton et al., 2016); the control group may be mice transferred with PBMC-purified iNKT cells. The pilot in vivo acute toxicity may be carried out by infusing naïve NSG mice with a low (10⁶) or a high (10⁷) dose iNKT cells. Mice (n=8) may then be observed 2 weeks for any alterations in body weight and food consumption, as well as any abnormal behaviors. After 2 weeks, mice may be euthanized and blood may be collected for blood hematology and blood serum chemistry analysis (UCSD murine hematology and coagulation core lab); various mouse tissues may be harvested and submitted to UCLA core for pathological analysis.

Allogeneic transplant-associated safety testing in vitro and in vivo The ^{U}HSC-iNKT therapy is of allogeneic transplant nature and thus its related safety may be evaluated. The potential of allogeneic reaction may be first determined by a standard two-way in vitro mixed lymphocyte reactions (MLR) assay (Bromelow et al., 2001). ^{U}HSC-iNKT cells may be mixed with mismatched donor PBMCs (at least three different donor batches) and T cell proliferation may be measured by the BrdU incorporation assay. For the study of GvHD, ^{U}HSC-iNKT may be the responder cells and PBMCs may be the stimulator cells; a reverse setting may be used to investigate HvG reactivity; stimulator cells will be irradiated prior to the incubation. One can also exploit an in vivo NSG mouse model to assess the in vivo GvHD and HvG reaction. Mice may be infused with ^{U}HSC-iNKT (5×10⁶, Group 1), human PBMCs (5×10⁶, Group 2), or combination (5×10⁶ each, Group 3). Mice may be observed for 2 months for any signs of toxicity (weight loss, behaviors, etc.). Mononuclear cells from bi-weekly mouse bleeding may be analyzed for human T cell activation markers (upregulation of hCD69 and hCD44, downregulation of hCD62L); ^{U}HSC-iNKT, human PBMC-derived CD8⁺ T, and human PBMC-derived CD4⁺ T cells may be identified by hCD45⁺6B11⁺, hCD45⁺6B11⁻ TCRαβ⁺CD8⁺, and hCD45⁺6B11⁻TCRαβCD4⁺, respectively. Compared to Groups 1 and 2, lack of activation of iNKT cells and lack of depletion of PBMCs in the Group 3 mice may indicate the lack of GvHD reactions, whereas lack of the activation of PBMC CD8/CD4 T cells and lack of depletion of ^{U}HSC-iNKT cells in the Group 3 mice may indicate the lack of HvG reactions.

Lentiviral vector safety and gene editing-related off-target analysis As a product releasing testing, the RCL assay may be measured to ensure patients not to be inadvertently exposed to replicating virus. One can also extract the genomic DNA from ^{U}HSC-iNKT cells and submit it for lentivirus integration site sequencing (Applied Biological Materials Inc.) to detect any unusual integrations other than the known lentiviral integration patterns. To analyze the gene editing-related off-target effect, one can use the CRISPR design tool from MIT to predict potential off-target sites and assess/confirm them by targeted re-sequencing of the genomic DNA of ^{U}HSC-iNKT cells. Additionally, one can perform unbiased genome-wide scans for off-target sites using GUILDE-seq in K562 cells electroporated with the Cas9/B2M-gRNA and Cas9/CIITA-gRNA RNPs and a dsODN tag (Tsai et al., 2015); these off-target sites may then be analyzed by NGS in ^{U}HSC-iNKT cells to detect the frequencies of off-target activity.

### Example 2: Anti-Tumor Efficacy of HSC-iNKT Cells Through an NK Cell-Like Path

### A. Pharmacology Study (FIG. 1)

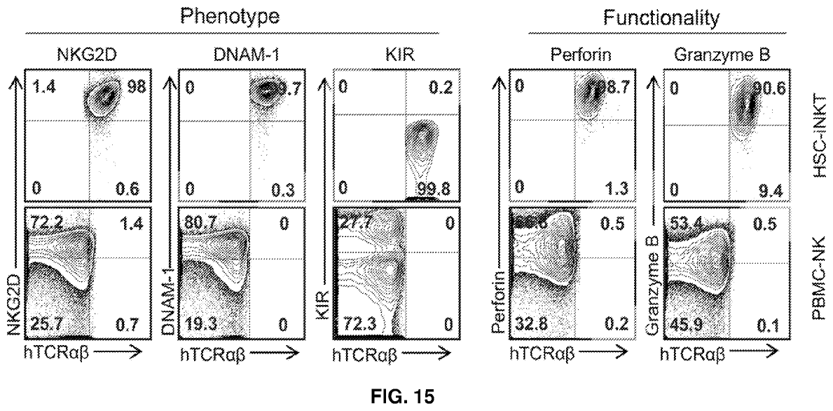

In vitro generated HSC-engineered iNKT (HSC-iNKT) cells displayed NK cell-like phenotype and functionality (FIG. 15). Interestingly, compared to native NK cells isolated from healthy donor PBMCs (PBMC-NK cells), HSC-iNKT cells expressed higher levels of NK activation receptors like NKG2D and DNAM-1, higher levels of cytotoxic molecules like Perforin and Granzyme B, while undetectable levels of NK inhibitory receptors like KIR (FIG. 15). These results suggest that HSC-iNKT cells may exhibit NK cell-like tumor cell targeting and killing capacity stronger than that of native NK cells.

### B. In Vitro Efficacy and MOA Study (FIG. 2)

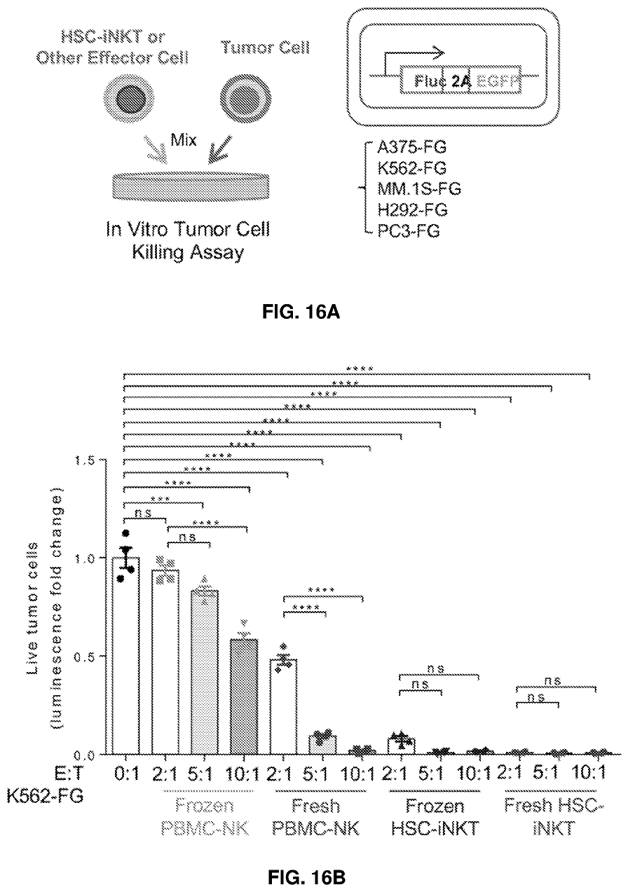

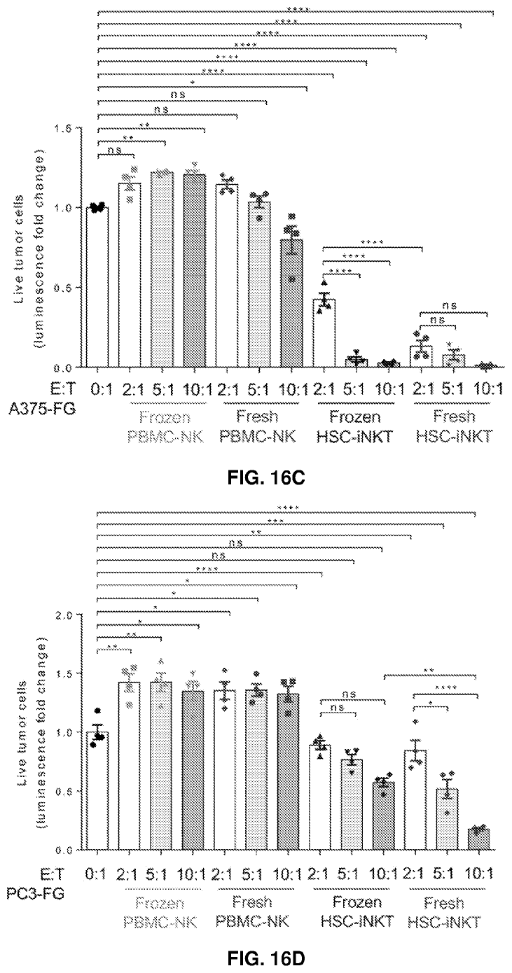

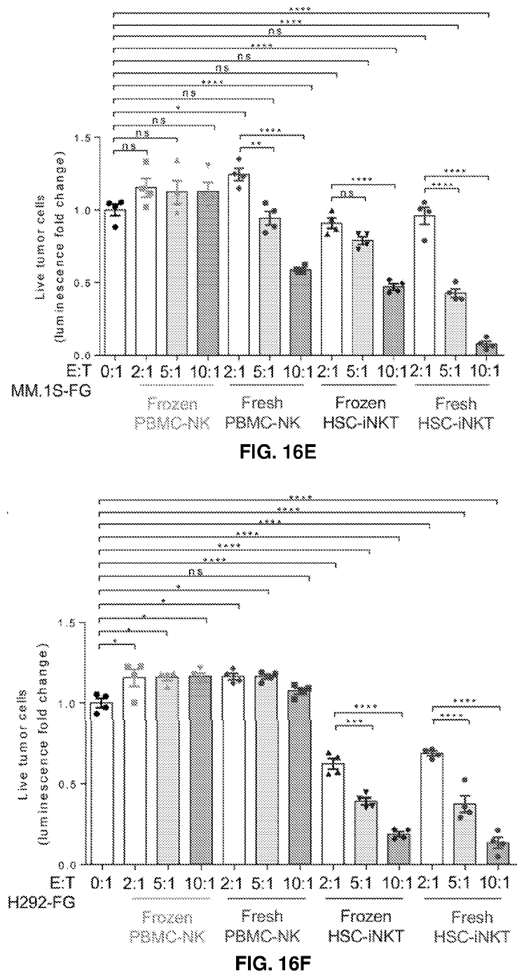

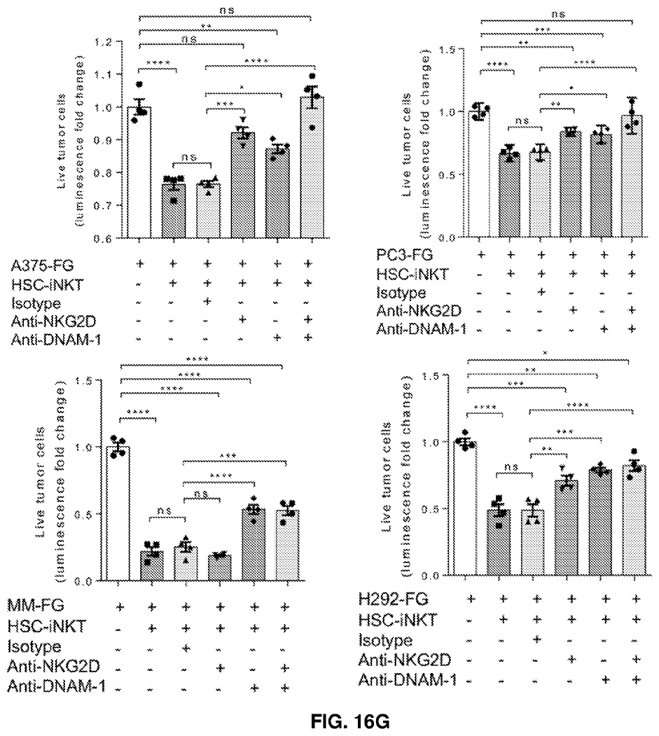

When studied using an in vitro tumor cell killing assay (FIG. 16A), HSC-iNKT cells showed enhanced killing of tumor cells that were sensitive to PBMC-NK cell killing, such as the K562 human chronic myelogenous leukemia cells (FIG. 16B). Most impressively, HSC-iNKT cells effectively killed multiple human blood cancer and solid tumor cell lines that were not sensitive to PBMC-NK cell killing, including the MM.1S human multiple myeloma cell line (FIG. 16E), the A375 human melanoma cell line (FIG. 16C), the PC3 human prostate cancer cell line (FIG. 16D), and the H292 human lung cancer cell line (FIG. 16F). Moreover, HSC-iNKT cells largely retained their tumor cell killing capacity post freeze/thaw cycle, unlike that of the PBMC-iNKT cells, suggesting that HSC-iNKT cells can be formulated as frozen cellular product for “off-the-shelf” therapy (FIG. 16B-2F). HSC-iNKT cell killing of these tumor cells were induced by stimulation of NK activation receptors, evidenced by the reduction of tumor cell killing efficacy by NKG2D and DNAM-1 blocking antibodies (FIG. 16G).

### C. In Vivo Efficacy and Safety Study (FIG. 3)

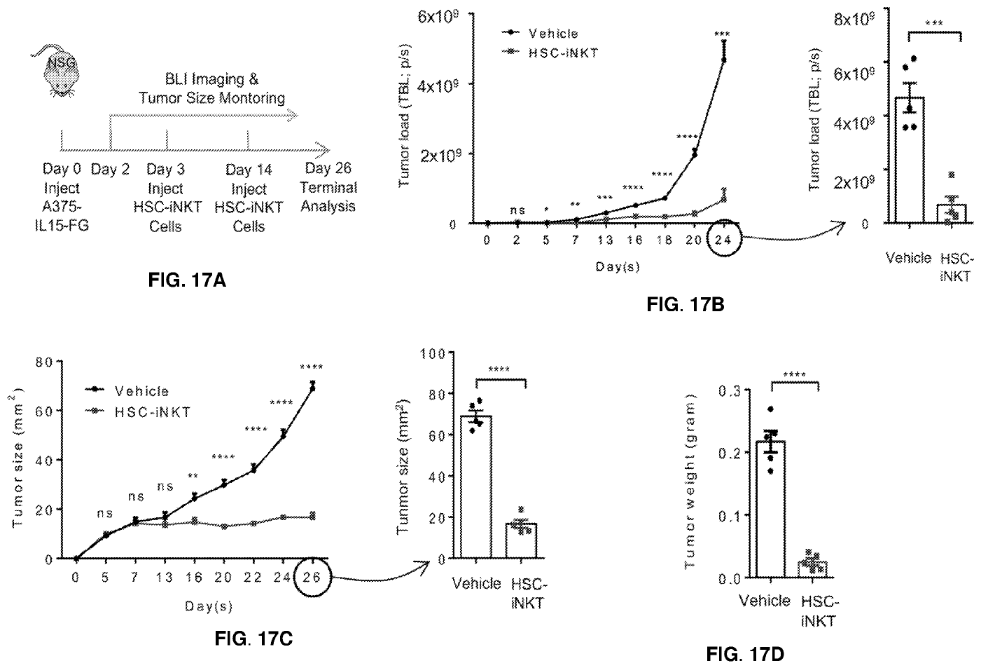

The in vivo anti-tumor efficacy of HSC-iNKT cells were studied using an A375-IL-15-FG human melanoma xenograft NSG mouse model (FIG. 17A). Adoptive transfer of HSC-iNKT cells significantly inhibited tumor growth (FIG. 17B-D). Importantly, no toxicity and tissue abnormality were observed in tumor-bearing animals receiving HSC-iNKT cell transfer, indicating the safety of HSC-iNKT cells.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.
Abrahimi, P., et al., Efficient gene disruption in cultured primary human endothelial cells by CRISPR/Cas9. Circ Res, 2015. 117(2): p. 121-8.
Bajgain, P., et al., Optimizing the production of suspension cells using the G-Rex “M” series. Molecular Therapy-Methods & Clinical Development, 2014. 1.
Bendelac, A., P. B. Savage, and L. Teyton, The biology of NKT cells. Annu Rev Immunol, 2007. 25: p. 297-336.
Berzins, S. P., M. J. Smyth, and A. G. Baxter, Presumed guilty: natural killer T cell defects and human disease. Nat Rev Immunol, 2011. 11(2): p. 131-42.
Bromelow, K. V., et al., Whole blood assay for assessment of the mixed lymphocyte reaction. Journal of Immunological Methods, 2001. 247(1-2): p. 1-8.
Chodon, T., et al., Adoptive Transfer of MART-1 T-Cell Receptor Transgenic Lymphocytes and Dendritic Cell Vaccination in Patients with Metastatic Melanoma. Clinical Cancer Research, 2014. 20(9): p. 2457-2465.
Cornetta, K., et al., Replication-competent Lentivirus Analysis of Clinical Grade Vector Products. Molecular Therapy, 2011. 19(3): p. 557-566.
de Lalla, C., et al., Invariant NKT cell reconstitution in pediatric leukemia patients given HLA-haploidentical stem cell transplantation defines distinct CD4+ and CD4− subset dynamics and correlates with remission state. J Immunol, 2011. 186(7): p. 4490-9.
Deotare, U., et al., G-CSF-primed bone marrow as a source of stem cells for allografting: revisiting the concept. Bone Marrow Transplantation, 2015. 50(9): p. 1150-1156.
Dudley, M. E., et al., Adoptive Cell Therapy for Patients With Metastatic Melanoma: Evaluation of Intensive Myeloablative Chemoradiation Preparative Regimens. Journal of Clinical Oncology, 2008. 26(32): p. 5233-5239.
Durgan, K., et al., Targeting NKT cells and PD-L1 pathway results in augmented anti-tumor responses in a melanoma model. Cancer Immunology Immunotherapy, 2011. 60(4): p. 547-558.
Fujii, S., et al., NKT cells as an ideal anti-tumor immunotherapeutic. Front Immunol, 2013. 4: p. 409.
Grupp, S. A., et al., Chimeric Antigen Receptor-Modified T Cells for Acute Lymphoid Leukemia. New England Journal of Medicine, 2013. 368(16): p. 1509-1518.
Gschweng, E. H., et al., HSV-sr39TK Positron Emission Tomography and Suicide Gene Elimination of Human Hematopoietic Stem Cells and Their Progeny in Humanized Mice. Cancer Research, 2014. 74(18): p. 5173-5183.
Gundry, M. C., et al., Highly Efficient Genome Editing of Murine and Human Hematopoietic Progenitor Cells by CRISPR/Cas9. Cell Reports, 2016. 17(5): p. 1453-1461.
Haraguchi, K., et al., Recovery of Valpha24+ NKT cells after hematopoietic stem cell transplantation. Bone Marrow Transplant, 2004. 34(7): p. 595-602.
Heczey, A., et al., Invariant NKT cells with chimeric antigen receptor provide a novel platform for safe and effective cancer immunotherapy. Blood, 2014. 124(18): p. 2824-33.
Horibata, S., et al., Utilization of the Soft Agar Colony Formation Assay to Identify Inhibitors of Tumorigenicity in Breast Cancer Cells. Jove-Journal of Visualized Experiments, 2015(99).
Hurton, L. V., et al., Tethered IL-15 augments antitumor activity and promotes a stem-cell memory subset in tumor-specific T cells. Proceedings of the National Academy of Sciences of the United States of America, 2016. 113(48): p. E7788-E7797.
Jin, J. J., et al., Simplified Method of the Growth of Human Tumor Infiltrating Lymphocytes in Gas permeable Flasks to Numbers Needed for Patient Treatment. Journal of Immunotherapy, 2012. 35(3): p. 283-292.
Kronenberg, M. and L. Gapin, The unconventional lifestyle of NKT cells. Nat Rev Immunol, 2002. 2(8): p. 557-68.
Liu, X. J., et al., CRISPR-Cas9-mediated multiplex gene editing in CAR-T cells. Cell Research, 2017. 27(1): p. 154-157.
Montoya, C. J., et al., Characterization of human invariant natural killer T subsets in health and disease using a novel invariant natural killer T cell-clonotypic monoclonal antibody, 6B11. Immunology, 2007. 122(1): p. 1-14.
Nolta, J. A., M. B. Hanley, and D. B. Kohn, Sustained human hematopoiesis in immunodeficient mice by cotransplantation of marrow stroma expressing human interleukin-3: analysis of gene transduction of long-lived progenitors. Blood, 1994. 83(10): p. 3041-51.
Pilones, K. A., J. Aryankalayil, and S. Demaria, Invariant NKT Cells as Novel Targets for Immunotherapy in Solid Tumors. Clinical & Developmental Immunology, 2012.
Ren, J. T., et al., Multiplex Genome Editing to Generate Universal CAR T Cells Resistant to PD1 Inhibition. Clinical Cancer Research, 2017. 23(9): p. 2255-2266.
Restifo, N. P., M. E. Dudley, and S. A. Rosenberg, Adoptive immunotherapy for cancer: harnessing the T cell response. Nat Rev Immunol, 2012. 12(4): p. 269-81.
Registry, C. C., http://www.ccrcal.org
Rosenberg, S. A., et al., Adoptive cell transfer: a clinical path to effective cancer immunotherapy. Nature Reviews Cancer, 2008. 8(4): p. 299-308.
Seet, C. S., et al., Generation of mature T cells from human hematopoietic stem and progenitor cells in artificial thymic organoids. Nature Methods, 2017. 14(5): p. 521-+.
Slaymaker, I. M., et al., Rationally engineered Cas9 nucleases with improved specificity. Science, 2016. 351(6268): p. 84-88.
Smith, D. J., et al., Genetic engineering of hematopoietic stem cells to generate invariant natural killer T cells. Proc Natl Acad Sci USA, 2015. 112(5): p. 1523-8.
Steimle, V., et al., Regulation of MHC class II expression by interferon-gamma mediated by the transactivator gene CIITA. Science, 1994. 265(5168): p. 106-9.
Sznol, M. and L. Chen, Antagonist antibodies to PD-1 and B7-H1 (PD-L1) in the treatment of advanced human cancer. Clin Cancer Res, 2013. 19(5): p. 1021-34.
Tian, G., et al., CD62L+ NKT cells have prolonged persistence and antitumor activity in vivo. J Clin Invest, 2016. 126(6): p. 2341-55.
Tsai, S. Q. and J. K. Joung, Defining and improving the genome-wide specificities of CRISPR-Cas9 nucleases. Nature Reviews Genetics, 2016. 17(5): p. 300-312.
Tsai, S. Q., et al., GUIDE-seq enables genome-wide profiling of off-target cleavage by CRISPR-Cas nucleases. Nature Biotechnology, 2015. 33(2): p. 187-197.
Yamasaki, K., et al., Induction of NKT cell-specific immune responses in cancer tissues after NKT cell-targeted adoptive immunotherapy. Clin Immunol, 2011. 138(3): p. 255-65.
Wu, C. F., et al., Development of an Inducible Caspase-9 Safety Switch for Pluripotent Stem Cell Based Therapies. Blood, 2012. 120(21
Vera, J. F., et al., Accelerated Production of Antigen-specific T Cells for Preclinical and Clinical Applications Using Gas permeable Rapid Expansion Cultureware (G-Rex). Journal of Immunotherapy, 2010. 33(3): p. 305-315.
Vivier, E., et al., Targeting natural killer cells and natural killer T cells in cancer. Nat Rev Immunol, 2012. 12(4): p. 239-52.
Watarai, H., et al., Methods for detection, isolation and culture of mouse and human invariant NKT cells. Nat Protoc, 2008. 3(1): p. 70-8.

### PATENTS AND PATENT APPLICATIONS

U.S. Pat. No. 5,843,780
U.S. Pat. No. 6,200,806
U.S. Pat. No. 6,506,559
U.S. Pat. No. 6,573,099
U.S. Pat. No. 6,833,269
U.S. Pat. No. 7,029,913
U.S. Pat. No. 7,795,404
U.S. Pat. No. 8,021,867
U.S. Pat. No. 8,377,886
U.S. Pat. No. 8,628,767
U.S. Patent Application Publication 2002/0168707
U.S. Patent Application Publication 2003/0159161
U.S. Patent Application Publication 2003/0022367
U.S. Patent Application Publication 2003/0051263
U.S. Patent Application Publication 2003/0055020
U.S. Patent Application Publication 2004/0014191
U.S. Patent Application Publication 2004/0265839
U.S. Patent Application Publication 2004/0064842
U.S. Patent Application Publication 2014/0369979
U.S. Patent Application Publication 2014/0242033
PCT Patent Application No. PCT/US94/09760
PCT Patent Application No. PCT/US94/08574
PCT Patent Application No. PCT/US94/10501

1. An engineered invariant natural killer T (iNKT) cell that expresses at least one invariant natural killer (iNKT) T-cell receptor (TCR) and one or both of (1) an exogenous suicide gene product; and (2) the genome of the cell has been altered to eliminate surface expression of at least one HLA-I or HLA-II molecule, wherein the at least one iNKT TCR is expressed from an exogenous nucleic acid and/or from an endogenous invariant TCR gene that is under the transcriptional control of a recombinantly modified promoter region.

2. The engineered iNKT cell of claim 1, wherein the genome of the cell has been altered to eliminate surface expression of at least one HLA-I or HLA-II molecule.

3. The engineered iNKT cell of claim 1 or 2, wherein the invariant TCR gene product is an alpha TCR gene product.

4. The engineered iNKT cell of claim 1, wherein the invariant TCR gene product is a beta TCR gene product.

5. The engineered iNKT cell of claim 1, wherein both an alpha TCR gene product and a beta TCR gene product are expressed.

6. The engineered iNKT cell of claim 1, wherein at least one invariant TCR gene product is expressed from an exogenous nucleic acid.

7. The engineered iNKT cell of claim 1, wherein the exogenous suicide gene product and/or the exogenous nucleic acid has one or more codons optimized for expression in the cell.

8. The engineered iNKT cell of claim 1, wherein suicide gene product is herpes simplex virus thymidine kinase (HSV-TK), purine nucleoside phosphorylase (PNP), cytosine deaminase (CD), carboxypetidase G2, cytochrome P450, linamarase, beta-lactamase, nitroreductase (NTR), carboxypeptidase A, or inducible caspase 9.

9-155. (canceled)

156. The engineered iNKT cell of claim 1, wherein the iNKT cells do not express surface HLA-I or -II molecules by disrupting the expression of genes encoding beta-2-microglobulin (B2M), major histocompatibility complex II transactivator (CIITA), and/or individual HLA-I and HLA-II molecules.

157. The engineered iNKT cell of claim 1, wherein the iNKT cell comprises a nucleic acid from a recombinant vector that was introduced into the cells

158. The engineered iNKT cell of claim 1, wherein the cell was not exposed to media comprises animal serum.

159. The engineered iNKT cell of claim 1, wherein the cell has previously been frozen and wherein the cell is stable at room temperature for at least one hour

160. The engineered iNKT cell of claim 1, wherein the suicide gene product is activated by a substrate.

161. The engineered iNKT cell of claim 1, wherein the cells comprise an exogenous nucleic acid encoding a polypeptide that has a substrate that may be labeled for imaging.

162. A method of preparing an engineered invariant natural killer T (iNKT) cell of claim 1 comprising:
a) selecting CD34+ cells from a plurality of hematopoietic stem or progenitor cells;
b) introducing one or more nucleic acids encoding at least one human invariant natural killer (iNKT) T-cell receptor (TCR);
c) eliminating surface expression of one or more HLA-I and/or HLA-II molecules in the isolated human CD34+ cells; and,
d) culturing isolated CD34+ cells expressing iNKT TCR to produce the invariant natural killer iNKT cell.

163. The method of claim 162, wherein culturing isolated CD34+ cells expressing iNKT TCR comprises culturing the CD34+ cells in an artificial thymic organoid (ATO) system to produce iNKT cells, wherein the ATO system comprises a 3D cell aggregate comprising a selected population of stromal cells that express a Notch ligand and a serum-free medium.

164. The method of claim 162, wherein the CD34+ cells are from a population comprising differentiated hematopoietic cells.

165. The method of claim 164, wherein the differentiated hematopoietic cells are peripheral blood mononuclear cells (PBMCs).

166. The method of claim 162, wherein the stem or progenitor cells comprise cord blood cells, fetal liver cells, embryonic stem cells, induced pluripotent stem cells, or bone marrow cells.

167. The method of claim 162, further comprising culturing selected CD34+ cells in media prior to introducing one or more nucleic acids into the cells.
